SEQUENCE LISTING (320 sequences)

SEQ ID NO: 1 - protein, 10 aa, Artificial Sequence
  Biologic
GGTFSSYAIS

SEQ ID NO: 2 - protein, 17 aa, Artificial Sequence
  Biologic
GIIPIFGTANYAQKFQG

SEQ ID NO: 3 - protein, 12 aa, Artificial Sequence
  Biologic
GLWEVRALPSVY

SEQ ID NO: 4 - protein, 11 aa, Artificial Sequence
  Biologic
GANDIGSKSVH

SEQ ID NO: 5 - protein, 7 aa, Artificial Sequence
  Biologic
EDIIRPS

SEQ ID NO: 6 - protein, 11 aa, Artificial Sequence
  Biologic
QVWDRDSDQYV

SEQ ID NO: 7 - protein, 121 aa, Artificial Sequence
  Biologic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 8 - protein, 109 aa, Artificial Sequence
  Biologic
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 9 - protein, 447 aa, Artificial Sequence
  Biologic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial Sequence
  Biologic
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 447 aa, Artificial Sequence
  Biologic
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 215 aa, Artificial Sequence
  Biologic
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 10 aa, Artificial Sequence
  Biologic
GYTFTTYWMH

SEQ ID NO: 14 - protein, 5 aa, Artificial Sequence
  Biologic
TYWMH

SEQ ID NO: 15 - protein, 17 aa, Artificial Sequence
  Biologic
NIYPGTGGSNFDEKFKN

SEQ ID NO: 16 - protein, 8 aa, Artificial Sequence
  Biologic
WTTGTGAY

SEQ ID NO: 17 - protein, 7 aa, Artificial Sequence
  Biologic
GYTFTTY

SEQ ID NO: 18 - protein, 6 aa, Artificial Sequence
  Biologic
YPGTGG

SEQ ID NO: 19 - protein, 117 aa, Artificial Sequence
  Biologic
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 20 - DNA, 351 bp, Artificial Sequence
  Biologic
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 21 - protein, 443 aa, Artificial Sequence
  Biologic
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - DNA, 1329 bp, Artificial Sequence
  Biologic
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 23 - protein, 17 aa, Artificial Sequence
  Biologic
KSSQSLLDSGNQKNFLT

SEQ ID NO: 24 - protein, 7 aa, Artificial Sequence
  Biologic
WASTRES

SEQ ID NO: 25 - protein, 9 aa, Artificial Sequence
  Biologic
QNDYSYPYT

SEQ ID NO: 26 - protein, 13 aa, Artificial Sequence
  Biologic
SQSLLDSGNQKNF

SEQ ID NO: 27 - protein, 3 aa, Artificial Sequence
  Biologic
WAS

SEQ ID NO: 28 - protein, 6 aa, Artificial Sequence
  Biologic
DYSYPY

SEQ ID NO: 29 - protein, 113 aa, Artificial Sequence
  Biologic
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 30 - DNA, 339 bp, Artificial Sequence
  Biologic
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 31 - protein, 220 aa, Artificial Sequence
  Biologic
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 660 bp, Artificial Sequence
  Biologic
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 33 - protein, 113 aa, Artificial Sequence
  Biologic
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 34 - DNA, 339 bp, Artificial Sequence
  Biologic
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 35 - protein, 220 aa, Artificial Sequence
  Biologic
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 660 bp, Artificial Sequence
  Biologic
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 37 - DNA, 15 bp, Artificial Sequence
  Biologic
acctactggatgcac

SEQ ID NO: 38 - DNA, 51 bp, Artificial Sequence
  Biologic
aatatctaccccggcaccggcggctctaacttcgacgagaagtttaagaat

SEQ ID NO: 39 - DNA, 24 bp, Artificial Sequence
  Biologic
tggactaccggcacaggcgcctac

SEQ ID NO: 40 - DNA, 21 bp, Artificial Sequence
  Biologic
ggctacaccttcactacctac

SEQ ID NO: 41 - DNA, 18 bp, Artificial Sequence
  Biologic
taccccggcaccggcggc

SEQ ID NO: 42 - DNA, 51 bp, Artificial Sequence
  Biologic
aaatctagtcagtcactgctggatagcggtaatcagaagaacttcctgacc

SEQ ID NO: 43 - DNA, 21 bp, Artificial Sequence
  Biologic
tgggcctctactagagaatca

SEQ ID NO: 44 - DNA, 27 bp, Artificial Sequence
  Biologic
cagaacgactatagctacccctacacc

SEQ ID NO: 45 - DNA, 39 bp, Artificial Sequence
  Biologic
agtcagtcactgctggatagcggtaatcagaagaacttc

SEQ ID NO: 46 - DNA, 9 bp, Artificial Sequence
  Biologic
tgggcctct

SEQ ID NO: 47 - DNA, 18 bp, Artificial Sequence
  Biologic
gactatagctacccctac

SEQ ID NO: 48 - protein, 440 aa, Artificial Sequence
  Biologic
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 214 aa, Artificial Sequence
  Biologic
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 447 aa, Artificial Sequence
  Biologic
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 218 aa, Artificial Sequence
  Biologic
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 447 aa, Artificial Sequence
  Biologic
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 213 aa, Artificial Sequence
  Biologic
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 5 aa, Artificial Sequence
  Biologic
SYWMY

SEQ ID NO: 55 - protein, 17 aa, Artificial Sequence
  Biologic
RIDPNSGSTKYNEKFKN

SEQ ID NO: 56 - protein, 11 aa, Artificial Sequence
  Biologic
DYRKGLYAMDY

SEQ ID NO: 57 - protein, 7 aa, Artificial Sequence
  Biologic
GYTFTSY

SEQ ID NO: 58 - protein, 6 aa, Artificial Sequence
  Biologic
DPNSGS

SEQ ID NO: 59 - protein, 120 aa, Artificial Sequence
  Biologic
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 60 - DNA, 360 bp, Artificial Sequence
  Biologic
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 61 - protein, 446 aa, Artificial Sequence
  Biologic
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 11 aa, Artificial Sequence
  Biologic
KASQDVGTAVA

SEQ ID NO: 63 - protein, 7 aa, Artificial Sequence
  Biologic
WASTRHT

SEQ ID NO: 64 - protein, 9 aa, Artificial Sequence
  Biologic
QQYNSYPLT

SEQ ID NO: 65 - protein, 7 aa, Artificial Sequence
  Biologic
SQDVGTA

SEQ ID NO: 66 - protein, 3 aa, Artificial Sequence
  Biologic
WAS

SEQ ID NO: 67 - protein, 6 aa, Artificial Sequence
  Biologic
YNSYPL

SEQ ID NO: 68 - DNA, 1338 bp, Artificial Sequence
  Biologic
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 69 - protein, 107 aa, Artificial Sequence
  Biologic
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 70 - DNA, 321 bp, Artificial Sequence
  Biologic
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 71 - protein, 214 aa, Artificial Sequence
  Biologic
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - DNA, 642 bp, Artificial Sequence
  Biologic
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 73 - protein, 120 aa, Artificial Sequence
  Biologic
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 74 - DNA, 360 bp, Artificial Sequence
  Biologic
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 75 - protein, 446 aa, Artificial Sequence
  Biologic
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - DNA, 1338 bp, Artificial Sequence
  Biologic
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 77 - protein, 107 aa, Artificial Sequence
  Biologic
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 78 - DNA, 321 bp, Artificial Sequence
  Biologic
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 79 - protein, 214 aa, Artificial Sequence
  Biologic
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 642 bp, Artificial Sequence
  Biologic
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 81 - DNA, 15 bp, Artificial Sequence
  Biologic
agctactggatgtac

SEQ ID NO: 82 - DNA, 51 bp, Artificial Sequence
  Biologic
agaatcgaccctaatagcggctctactaagtataacgagaagtttaagaat

SEQ ID NO: 83 - DNA, 33 bp, Artificial Sequence
  Biologic
gactatagaaagggcctgtacgctatggactac

SEQ ID NO: 84 - DNA, 21 bp, Artificial Sequence
  Biologic
ggctacaccttcactagctac

SEQ ID NO: 85 - DNA, 18 bp, Artificial Sequence
  Biologic
gaccctaatagcggctct

SEQ ID NO: 86 - DNA, 33 bp, Artificial Sequence
  Biologic
aaagcctctcaggacgtgggcaccgccgtggcc

SEQ ID NO: 87 - DNA, 21 bp, Artificial Sequence
  Biologic
tgggcctctactagacacacc

SEQ ID NO: 88 - DNA, 27 bp, Artificial Sequence
  Biologic
cagcagtataatagctaccccctgacc

SEQ ID NO: 89 - DNA, 21 bp, Artificial Sequence
  Biologic
tctcaggacgtgggcaccgcc

SEQ ID NO: 90 - DNA, 9 bp, Artificial Sequence
  Biologic
tgggcctct

SEQ ID NO: 91 - DNA, 18 bp, Artificial Sequence
  Biologic
tataatagctaccccctg

SEQ ID NO: 92 - protein, 448 aa, Artificial Sequence
  Biologic
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 214 aa, Artificial Sequence
  Biologic
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, Artificial Sequence
  Biologic
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 216 aa, Artificial Sequence
  Biologic
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 96 - protein, 451 aa, Artificial Sequence
  Biologic
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 215 aa, Artificial Sequence
  Biologic
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 123 aa, Artificial Sequence
  Biologic
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 99 - protein, 106 aa, Artificial Sequence
  Biologic
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 100 - protein, 10 aa, Artificial Sequence
  Biologic
GYTFTSYWMY

SEQ ID NO: 101 - protein, 121 aa, Artificial Sequence
  Biologic
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 102 - protein, 107 aa, Artificial Sequence
  Biologic
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 103 - protein, 451 aa, Artificial Sequence
  Biologic
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, Artificial Sequence
  Biologic
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 363 bp, Artificial Sequence
  Biologic
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 106 - DNA, 321 bp, Artificial Sequence
  Biologic
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 107 - DNA, 1353 bp, Artificial Sequence
  Biologic
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 108 - DNA, 642 bp, Artificial Sequence
  Biologic
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 109 - protein, 5 aa, Artificial Sequence
  Biologic
SYGVD

SEQ ID NO: 110 - protein, 7 aa, Artificial Sequence
  Biologic
GFSLSSY

SEQ ID NO: 111 - protein, 16 aa, Artificial Sequence
  Biologic
VIWGGGGTYYASSLMG

SEQ ID NO: 112 - protein, 5 aa, Artificial Sequence
  Biologic
WGGGG

SEQ ID NO: 113 - protein, 13 aa, Artificial Sequence
  Biologic
HAYGHDGGFAMDY

SEQ ID NO: 114 - protein, 11 aa, Artificial Sequence
  Biologic
RASESVSSNVA

SEQ ID NO: 115 - protein, 7 aa, Artificial Sequence
  Biologic
SESVSSN

SEQ ID NO: 116 - protein, 7 aa, Artificial Sequence
  Biologic
GASNRAT

SEQ ID NO: 117 - protein, 3 aa, Artificial Sequence
  Biologic
GAS

SEQ ID NO: 118 - protein, 9 aa, Artificial Sequence
  Biologic
GQSYSYPFT

SEQ ID NO: 119 - protein, 6 aa, Artificial Sequence
  Biologic
SYSYPF

SEQ ID NO: 120 - protein, 124 aa, Artificial Sequence
  Biologic
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 121 - protein, 107 aa, Artificial Sequence
  Biologic
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 122 - protein, 10 aa, Artificial Sequence
  Biologic
GFTLTNYGMN

SEQ ID NO: 123 - protein, 5 aa, Artificial Sequence
  Biologic
NYGMN

SEQ ID NO: 124 - protein, 17 aa, Artificial Sequence
  Biologic
WINTDTGEPTYADDFKG

SEQ ID NO: 125 - protein, 16 aa, Artificial Sequence
  Biologic
NPPYYYGTNNAEAMDY

SEQ ID NO: 126 - protein, 7 aa, Artificial Sequence
  Biologic
GFTLTNY

SEQ ID NO: 127 - protein, 6 aa, Artificial Sequence
  Biologic
NTDTGE

SEQ ID NO: 128 - protein, 125 aa, Artificial Sequence
  Biologic
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 129 - DNA, 375 bp, Artificial Sequence
  Biologic
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 130 - DNA, 375 bp, Artificial Sequence
  Biologic
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 131 - protein, 451 aa, Artificial Sequence
  Biologic
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 132 - protein, 11 aa, Artificial Sequence
  Biologic
SSSQDISNYLN

SEQ ID NO: 133 - protein, 7 aa, Artificial Sequence
  Biologic
YTSTLHL

SEQ ID NO: 134 - protein, 9 aa, Artificial Sequence
  Biologic
QQYYNLPWT

SEQ ID NO: 135 - protein, 7 aa, Artificial Sequence
  Biologic
SQDISNY

SEQ ID NO: 136 - protein, 3 aa, Artificial Sequence
  Biologic
YTS

SEQ ID NO: 137 - protein, 6 aa, Artificial Sequence
  Biologic
YYNLPW

SEQ ID NO: 138 - DNA, 1353 bp, Artificial Sequence
  Biologic
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 139 - DNA, 1353 bp, Artificial Sequence
  Biologic
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 140 - protein, 107 aa, Artificial Sequence
  Biologic
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 141 - DNA, 321 bp, Artificial Sequence
  Biologic
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 142 - DNA, 321 bp, Artificial Sequence
  Biologic
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 143 - protein, 214 aa, Artificial Sequence
  Biologic
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 642 bp, Artificial Sequence
  Biologic
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 145 - DNA, 642 bp, Artificial Sequence
  Biologic
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 146 - protein, 125 aa, Artificial Sequence
  Biologic
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 147 - DNA, 375 bp, Artificial Sequence
  Biologic
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 148 - DNA, 375 bp, Artificial Sequence
  Biologic
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 149 - protein, 451 aa, Artificial Sequence
  Biologic
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 150 - DNA, 1353 bp, Artificial Sequence
  Biologic
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 151 - DNA, 1353 bp, Artificial Sequence
  Biologic
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 152 - protein, 107 aa, Artificial Sequence
  Biologic
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 153 - DNA, 321 bp, Artificial Sequence
  Biologic
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 154 - DNA, 321 bp, Artificial Sequence
  Biologic
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 155 - protein, 214 aa, Artificial Sequence
  Biologic
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - DNA, 642 bp, Artificial Sequence
  Biologic
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 157 - DNA, 642 bp, Artificial Sequence
  Biologic
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 158 - DNA, 15 bp, Artificial Sequence
  Biologic
aattacgggatgaac

SEQ ID NO: 159 - DNA, 15 bp, Artificial Sequence
  Biologic
aactacggcatgaac

SEQ ID NO: 160 - DNA, 51 bp, Artificial Sequence
  Biologic
tggattaacaccgacaccggggagcctacctacgcggacgatttcaaggga

SEQ ID NO: 161 - DNA, 51 bp, Artificial Sequence
  Biologic
tggatcaacaccgacaccggcgagcctacctacgccgacgacttcaagggc

SEQ ID NO: 162 - DNA, 48 bp, Artificial Sequence
  Biologic
aacccgccctactactacggaaccaacaacgccgaagccatggactac

SEQ ID NO: 163 - DNA, 48 bp, Artificial Sequence
  Biologic
aaccccccttactactacggcaccaacaacgccgaggccatggactat

SEQ ID NO: 164 - DNA, 21 bp, Artificial Sequence
  Biologic
ggattcaccctcaccaattac

SEQ ID NO: 165 - DNA, 21 bp, Artificial Sequence
  Biologic
ggcttcaccctgaccaactac

SEQ ID NO: 166 - DNA, 18 bp, Artificial Sequence
  Biologic
aacaccgacaccggggag

SEQ ID NO: 167 - DNA, 18 bp, Artificial Sequence
  Biologic
aacaccgacaccggcgag

SEQ ID NO: 168 - DNA, 33 bp, Artificial Sequence
  Biologic
agctctagtcaggatatctctaactacctgaac

SEQ ID NO: 169 - DNA, 33 bp, Artificial Sequence
  Biologic
tcctccagccaggacatctccaactacctgaac

SEQ ID NO: 170 - DNA, 21 bp, Artificial Sequence
  Biologic
tacactagcaccctgcacctg

SEQ ID NO: 171 - DNA, 21 bp, Artificial Sequence
  Biologic
tacacctccaccctgcacctg

SEQ ID NO: 172 - DNA, 27 bp, Artificial Sequence
  Biologic
cagcagtactataacctgccctggacc

SEQ ID NO: 173 - DNA, 27 bp, Artificial Sequence
  Biologic
cagcagtactacaacctgccctggacc

SEQ ID NO: 174 - DNA, 21 bp, Artificial Sequence
  Biologic
agtcaggatatctctaactac

SEQ ID NO: 175 - DNA, 21 bp, Artificial Sequence
  Biologic
agccaggacatctccaactac

SEQ ID NO: 176 - DNA, 9 bp, Artificial Sequence
  Biologic
tacactagc

SEQ ID NO: 177 - DNA, 9 bp, Artificial Sequence
  Biologic
tacacctcc

SEQ ID NO: 178 - DNA, 18 bp, Artificial Sequence
  Biologic
tactataacctgccctgg

SEQ ID NO: 179 - DNA, 18 bp, Artificial Sequence
  Biologic
tactacaacctgccctgg

SEQ ID NO: 180 - DNA, 15 bp, Artificial Sequence
  Biologic
aactacgggatgaac

SEQ ID NO: 181 - DNA, 51 bp, Artificial Sequence
  Biologic
tggattaacaccgacaccggcgagcctacctacgccgacgactttaagggc

SEQ ID NO: 182 - DNA, 48 bp, Artificial Sequence
  Biologic
aaccccccctactactacggcactaacaacgccgaggctatggactac

SEQ ID NO: 183 - DNA, 21 bp, Artificial Sequence
  Biologic
ggcttcaccctgactaactac

SEQ ID NO: 184 - protein, 447 aa, Artificial Sequence
  Biologic
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 214 aa, Artificial Sequence
  Biologic
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 446 aa, Artificial Sequence
  Biologic
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 220 aa, Artificial Sequence
  Biologic
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 10 aa, Artificial Sequence
  Biologic
GFTLTNYGMN

SEQ ID NO: 189 - protein, 5 aa, Artificial Sequence
  Biologic
SYNMH

SEQ ID NO: 190 - protein, 17 aa, Artificial Sequence
  Biologic
DIYPGNGDTSYNQKFKG

SEQ ID NO: 191 - protein, 9 aa, Artificial Sequence
  Biologic
VGGAFPMDY

SEQ ID NO: 192 - protein, 7 aa, Artificial Sequence
  Biologic
GYTFTSY

SEQ ID NO: 193 - protein, 6 aa, Artificial Sequence
  Biologic
YPGNGD

SEQ ID NO: 194 - protein, 118 aa, Artificial Sequence
  Biologic
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 195 - DNA, 354 bp, Artificial Sequence
  Biologic
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 196 - protein, 444 aa, Artificial Sequence
  Biologic
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 197 - DNA, 1332 bp, Artificial Sequence
  Biologic
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 198 - protein, 15 aa, Artificial Sequence
  Biologic
RASESVEYYGTSLMQ

SEQ ID NO: 199 - protein, 7 aa, Artificial Sequence
  Biologic
AASNVES

SEQ ID NO: 200 - protein, 9 aa, Artificial Sequence
  Biologic
QQSRKDPST

SEQ ID NO: 201 - protein, 11 aa, Artificial Sequence
  Biologic
SESVEYYGTSL

SEQ ID NO: 202 - protein, 3 aa, Artificial Sequence
  Biologic
AAS

SEQ ID NO: 203 - protein, 6 aa, Artificial Sequence
  Biologic
SRKDPS

SEQ ID NO: 204 - protein, 111 aa, Artificial Sequence
  Biologic
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 205 - DNA, 333 bp, Artificial Sequence
  Biologic
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 206 - protein, 218 aa, Artificial Sequence
  Biologic
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 654 bp, Artificial Sequence
  Biologic
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 208 - protein, 17 aa, Artificial Sequence
  Biologic
DIYPGQGDTSYNQKFKG

SEQ ID NO: 209 - protein, 6 aa, Artificial Sequence
  Biologic
YPGQGD

SEQ ID NO: 210 - protein, 118 aa, Artificial Sequence
  Biologic
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 211 - DNA, 354 bp, Artificial Sequence
  Biologic
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 212 - protein, 444 aa, Artificial Sequence
  Biologic
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 213 - DNA, 1332 bp, Artificial Sequence
  Biologic
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 214 - protein, 111 aa, Artificial Sequence
  Biologic
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 215 - DNA, 333 bp, Artificial Sequence
  Biologic
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 216 - protein, 218 aa, Artificial Sequence
  Biologic
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - DNA, 654 bp, Artificial Sequence
  Biologic
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 218 - protein, 114 aa, Artificial Sequence
  Biologic
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 219 - protein, 108 aa, Artificial Sequence
  Biologic
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 220 - protein, 120 aa, Artificial Sequence
  Biologic
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 221 - protein, 113 aa, Artificial Sequence
  Biologic
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 222 - protein, 114 aa, Artificial Sequence
  Biologic
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 223 - protein, 170 aa, Artificial Sequence
  Biologic
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 224 - protein, 114 aa, Artificial Sequence
  Biologic
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 225 - protein, 297 aa, Artificial Sequence
  Biologic
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 114 aa, Artificial Sequence; identical to SEQ ID NO: 222
  Biologic

SEQ ID NO: 227 - protein, 77 aa, Artificial Sequence
  Biologic
ITCPPPMSVEHADIWVKSYSLYSRERYICNSGFKRKAGTSSLTECVLNKATNVAHWTTPSLKCIRDPALVHQRPAPP

SEQ ID NO: 228 - protein, 248 aa, Artificial Sequence
  Biologic
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGSTSGSGKPGSGEGSTKGELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 229 - protein, 111 aa, Artificial Sequence
  Biologic
ELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 230 - protein, 20 aa, Artificial Sequence
  Biologic
GGGGSGGGGSGGGGSGGGGS

SEQ ID NO: 231 - protein, 15 aa, Artificial Sequence
  Biologic
GGGGSGGGGSGGGGS

SEQ ID NO: 232 - protein, 4 aa, Artificial Sequence
  Biologic
GGGS

SEQ ID NO: 233 - protein, 242 aa, Artificial Sequence
  Biologic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 234 - protein, 242 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 235 - protein, 242 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 236 - protein, 242 aa, Artificial Sequence
  Biologic
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 237 - protein, 242 aa, Artificial Sequence
  Biologic
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 238 - protein, 247 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 239 - protein, 247 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 240 - protein, 247 aa, Artificial Sequence
  Biologic
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 241 - protein, 247 aa, Artificial Sequence
  Biologic
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 242 - protein, 247 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 243 - protein, 247 aa, Artificial Sequence
  Biologic
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 244 - protein, 242 aa, Artificial Sequence
  Biologic
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 245 - protein, 242 aa, Artificial Sequence
  Biologic
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 246 - protein, 12 aa, Artificial Sequence
  Biologic
HYYYGGSYAMDY

SEQ ID NO: 247 - protein, 9 aa, Artificial Sequence
  Biologic
QQGNTLPYT

SEQ ID NO: 248 - protein, 18 aa, Artificial Sequence
  Biologic
GSTSGSGKPGSGEGSTKG

SEQ ID NO: 249 - protein, 119 aa, Artificial Sequence
  Biologic
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVT

SEQ ID NO: 250 - protein, 7 aa, Artificial Sequence
  Biologic
HTSRLHS

SEQ ID NO: 251 - protein, 11 aa, Artificial Sequence
  Biologic
RASQDISKYLN

SEQ ID NO: 252 - protein, 486 aa, Artificial Sequence
  Biologic
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 253 - protein, 486 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 254 - protein, 486 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 255 - protein, 486 aa, Artificial Sequence
  Biologic
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 256 - protein, 486 aa, Artificial Sequence
  Biologic
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 257 - protein, 491 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - protein, 491 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 259 - protein, 491 aa, Artificial Sequence
  Biologic
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 260 - protein, 491 aa, Artificial Sequence
  Biologic
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 261 - protein, 491 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 262 - protein, 491 aa, Artificial Sequence; identical to SEQ ID NO: 261
  Biologic

SEQ ID NO: 263 - protein, 491 aa, Artificial Sequence
  Biologic
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 264 - protein, 486 aa, Artificial Sequence
  Biologic
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 45 aa, Artificial Sequence
  Biologic
TTTPAPRPPTPAPTIASQPLSLRPEACRPAAGGAVHTRGLDFACD

SEQ ID NO: 266 - protein, 24 aa, Artificial Sequence
  Biologic
IYIWAPLAGTCGVLLLSLVITLYC

SEQ ID NO: 267 - DNA, 72 bp, Artificial Sequence
  Biologic
atctacatctgggcgcccttggccgggacttgtggggtccttctcctgtcactggttatcaccctttactgc

SEQ ID NO: 268 - protein, 230 aa, Artificial Sequence
  Biologic
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 269 - DNA, 135 bp, Artificial Sequence
  Biologic
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 270 - DNA, 690 bp, Artificial Sequence
  Biologic
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 271 - protein, 282 aa, Artificial Sequence
  Biologic
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 272 - DNA, 847 bp, Artificial Sequence
  Biologic
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 273 - protein, 10 aa, Artificial Sequence
  Biologic
GGGGSGGGGS

SEQ ID NO: 274 - DNA, 30 bp, Artificial Sequence
  Biologic
ggtggcggaggttctggaggtggaggttcc

SEQ ID NO: 275 - protein, 112 aa, Artificial Sequence
  Biologic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 276 - protein, 112 aa, Artificial Sequence
  Biologic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 277 - DNA, 336 bp, Artificial Sequence
  Biologic
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 278 - DNA, 336 bp, Artificial Sequence
  Biologic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 279 - protein, 42 aa, Artificial Sequence
  Biologic
KRGRKKLLYIFKQPFMRPVQTTQEEDGCSCRFPEEEEGGCEL

SEQ ID NO: 280 - protein, 48 aa, Artificial Sequence
  Biologic
QRRKYRSNKGESPVEPAEPCRYSCPREEEGSTIPIQEDYRKPEPACSP

SEQ ID NO: 281 - DNA, 126 bp, Artificial Sequence
  Biologic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 282 - DNA, 144 bp, Artificial Sequence
  Biologic
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 283 - protein, 21 aa, Artificial Sequence
  Biologic
MALPVTALLLPLALLLHAARP

SEQ ID NO: 284 - protein, 16 aa, Artificial Sequence
  Biologic
VIWGSETTYYNSSLKS

SEQ ID NO: 285 - DNA, 1184 bp, Artificial Sequence
  Biologic
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 286 - protein, 448 aa, Artificial Sequence
  Biologic
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 214 aa, Artificial Sequence
  Biologic
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 6 aa, Artificial Sequence
  Biologic
SDYAWN

SEQ ID NO: 289 - protein, 16 aa, Artificial Sequence
  Biologic
YISYSGSTSYNPSLKS

SEQ ID NO: 290 - protein, 9 aa, Artificial Sequence
  Biologic
FDYAHAMDY

SEQ ID NO: 291 - protein, 11 aa, Artificial Sequence
  Biologic
QASQSIGTSIH

SEQ ID NO: 292 - protein, 7 aa, Artificial Sequence
  Biologic
YASESIS

SEQ ID NO: 293 - protein, 9 aa, Artificial Sequence
  Biologic
QQINSWPTT

SEQ ID NO: 294 - protein, 143 aa, Artificial Sequence
  Biologic
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDITSASYTMI

SEQ ID NO: 295 - protein, 16 aa, Artificial Sequence
  Biologic
VIWGSETTYYQSSLKS

SEQ ID NO: 296 - protein, 16 aa, Artificial Sequence
  Biologic
VIWGSETTYYSSSLKS

SEQ ID NO: 297 - protein, 137 aa, Artificial Sequence
  Biologic
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 298 - protein, 126 aa, Artificial Sequence
  Biologic
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK

SEQ ID NO: 299 - DNA, 63 bp, Artificial Sequence
  Biologic
atggccctgcctgtgacagccctgctgctgcctctggctctgctgctgcatgccgctagaccc

SEQ ID NO: 300 - DNA, 63 bp, Artificial Sequence
  Biologic
atggccctccctgtcaccgccctgctgcttccgctggctcttctgctccacgccgctcggccc

SEQ ID NO: 301 - DNA, 72 bp, Artificial Sequence
  Biologic
atctacatttgggcccctctggctggtacttgcggggtcctgctgctttcactcgtgatcactctttactgt

SEQ ID NO: 302 - DNA, 126 bp, Artificial Sequence
  Biologic
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 303 - DNA, 336 bp, Artificial Sequence
  Biologic
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 304 - protein, 5 aa, Artificial Sequence
  Biologic
GGGGS

SEQ ID NO: 305 - protein, 150 aa, Artificial Sequence [sequence omitted]

SEQ ID NO: 306 - DNA, 450 bp, Artificial Sequence
  Biologic
cccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtc

SEQ ID NO: 307 - protein, 394 aa, Artificial Sequence
  Biologic
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 308 - DNA, 1182 bp, Artificial Sequence
  Biologic
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 309 - protein, 4 aa, Artificial Sequence
  Biologic
GGGS

SEQ ID NO: 310 - RNA, 50 nt, Artificial Sequence
  Biologic
aaaaaaaaaaaaaaaaaaaaaaaaaaaaaaaaaaaaaaaaaaaaaaaaaa

SEQ ID NO: 311 - protein, 373 aa, Artificial Sequence
  Biologic
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 312 - protein, 35 aa, Artificial Sequence
  Biologic
TKKKYSSSVHDPNGEYMFMRAVNTAKKSRLTDVTL

SEQ ID NO: 313 - DNA, 105 bp, Artificial Sequence
  Biologic
acaaaaaagaagtattcatccagtgtgcacgaccctaacggtgaatacatgttcatgagagcagtgaacacagccaaaaaatccagactcacagatgtgacccta

SEQ ID NO: 314 - protein, 69 aa, Artificial Sequence
  Biologic
TTTPAPRPPTPAPTIASQPLSLRPEACRPAAGGAVHTRGLDFACDFWLPIGCAAFVVVCILGCILICWL

SEQ ID NO: 315 - DNA, 207 bp, Artificial Sequence
  Biologic
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggttacccataggatgtgcagcctttgttgtagtctgcattttgggatgcatacttatttgttggctt

SEQ ID NO: 316 - protein, 41 aa, Artificial Sequence
  Biologic
RSKRSRLLHSDYMNMTPRRPGPTRKHYQPYAPPRDFAAYRS

SEQ ID NO: 317 - DNA, 123 bp, Artificial Sequence
  Biologic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 318 - protein, 22 aa, Artificial Sequence
  Biologic
YGRKKRRQRRRLYRSPAMPENL

SEQ ID NO: 319 - protein, 10 aa, Artificial Sequence
  Biologic
GVSLPDYGVS

SEQ ID NO: 320 - protein, 16 aa, Artificial Sequence
  Biologic
VIWGSETTYYNSALKS

(19) United States
(12) Patent Application Publication
(10) Pub. No.: US 2023/0056470 A1
(43) Pub. Date: Feb. 23, 2023

(54) USES OF ANTI-TGF-BETA ANTIBODIES AND CHECKPOINT INHIBITORS FOR THE TREATMENT OF PROLIFERATIVE DISEASES

(71) Applicant: NOVARTIS AG, Basel (CH)

(72) Inventors: Mirek DOSTALEK, Binningen (CH); Claire FABRE, Basel (CH); Fariba KHANSHAN, Brookline, MA (US)

(21) Appl. No.: 17/786,265

(22) PCT Filed: Dec. 3, 2020
(86) PCT No.: PCT/IB2020/061458

§ 371 Date: Jun. 16, 2022

Related U.S. Application Data
(60) Provisional application No. 62/951,632, filed on Dec. 20, 2019.
(60) Provisional application No. 62/978,267, filed on Feb. 18, 2020.
(60) Provisional application No. 63/055,230, filed on Jul. 22, 2020.
(60) Provisional application No. 63/090,259, filed on Oct. 11, 2020.
(60) Provisional application No. 63/090,264, filed on Oct. 11, 2020.
(60) Provisional application No. 63/117,206, filed on Nov. 23, 2020.

(51) Int. Cl. C07K 16/22; C07K 16/28; A61P 35/00; A61K 45/06; A61K 39/395

(52) U.S. Cl. CPC C07K 16/22; C07K 16/2818; A61P 35/00; A61K 45/06; A61K 39/3955; A61K 2039/505

(57) ABSTRACT

Therapies using TGF-β inhibitors and/or PD-1 inhibitors are disclosed. These drugs at certain doses (including flat dosing) and regimens can be used to treat or prevent proliferative diseases such as solid tumors, including pancreatic cancers. Further combination and uses thereof are also disclosed.

### CROSS REFERENCE

This application claim the priority benefit of U.S. Provisional Application No. 62/951,632, filed Dec. 20, 2019, U.S. Provisional Application No. 62/978,267 filed on Feb. 18, 2020, U.S. Provisional Application No. 63/055,230 filed on Jul. 22, 2020, U.S. Provisional Application No. 63/090,259 filed on Oct. 11, 2020, U.S. Provisional Application No. 63/090,264 filed on Oct. 11, 2020 and U.S. Provisional Application No. 63/117,206, filed on Nov. 23, 2020, the contents of all applications of which are incorporated by reference in their entireties.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification controls.

### SEQUENCE LISTING

This application contains a Sequence Listing which has been submitted electronically in ASCII format and is hereby incorporated by reference in its entirety. The ASCII copy, created on Nov. 29, 2020, is named PAT058932_SL.txt and is 360,965 bytes in size.

### BACKGROUND

The transforming growth factor beta (TGFβ) protein family consists of three distinct isoforms found in mammals (TGFβ1, TGFβ2, and TGFβ3). The TGFβ proteins activate and regulate multiple gene responses that influence disease states, including cell proliferative, inflammatory, and cardiovascular conditions. TGFβ is a multifunctional cytokine originally named for its ability to transform normal fibroblasts to cells capable of anchorage-independent growth. The TGFβ molecules are produced primarily by hematopoietic and tumor cells and can regulate, i.e., stimulate or inhibit, the growth and differentiation of cells from a variety of both normal and neoplastic tissue origins (Sporn et al., Science, 233: 532 (1986)), and stimulate the formation and expansion of various stromal cells.

The TGFβs are known to be involved in many proliferative and non-proliferative cellular processes such as cell proliferation and differentiation, embryonic development, extracellular matrix formation, bone development, wound healing, hematopoiesis, and immune and inflammatory responses. See e.g., Pircher et al, Biochem. Biophys. Res. Commun., 136: 30-37 (1986); Wakefield et al., Growth Factors, 1: 203-218 (1989); Roberts and Sporn, pp 419-472 in Handbook of Experimental Pharmacology eds M. B. Sporn & A. B. Roberts (Springer, Heidelberg, 1990); Massague et al., Annual Rev. Cell Biol., 6: 597-646 (1990); Singer and Clark, New Eng. J. Med., 341: 738-745 (1999). Also, TGFβ is used in the treatment and prevention of diseases of the intestinal mucosa (WO 2001/24813). TGFβ is also known to have strong immunosuppressive effects on various immunologic cell types, including cytotoxic T lymphocyte (CTL) inhibition (Ranges et al., J. Exp. Med., 166: 991, 1987), Espevik et al., J. Immunol., 140: 2312, 1988), depressed B cell lymphopoiesis and kappa light-chain expression (Lee et al., J. Exp. Med., 166: 1290, 1987), negative regulation of hematopoiesis (Sing et al., Blood, 72: 1504, 1988), down-regulation of HLA-DR expression on tumor cells (Czarniecki et al., J. Immunol., 140: 4217, 1988), and inhibition of the proliferation of antigen-activated B lymphocytes in response to B-cell growth factor (Petit-Koskas et al., Eur. J. Immunol., 18: 111, 1988). See also U.S. Pat. No. 7,527,791.

There is an unmet need for using TGFβ inhibitors, such as anti-TGFβ antibodies, to target various diseases and medical conditions. Further, there is a need to administer these TGFβ inhibitors in such a way to effectively treat various diseases and medical conditions (including proliferative diseases) while maintaining dosing convenience.

### SUMMARY

Disclosed herein are methods of treating a proliferative disease in a subject. Treating the proliferative disease can comprise administering to a subject a TGF-β inhibitor at a dose of about 16 mg/kg to about 50 mg/kg. In some embodiments, the subject can be aware that they have the proliferative disease, e.g., is a subject in need thereof. In some embodiments, the TGF-β inhibitor comprises a heavy chain CDR1, CDR2, and CDR3, of SEQ ID NOs: 1, 2, and 3, respectfully, and a light chain CDR1, CDR2, and CDR3, of SEQ ID NOs: 4, 5, and 6, respectfully. Further, in some embodiments, the TGF-β inhibitor comprises a heavy chain variable region and the light chain variable region set out in amino acid sequence SEQ ID NOs: 7 and 8, respectively. In some embodiments, the TGF-β inhibitor comprises the heavy chain and light chain set out in amino acid sequence of SEQ ID NOs: 9 and 10, respectively. In some embodiments, the TGF-β inhibitor consists essentially of the heavy chain and light chain set out in amino acid sequence of SEQ ID NOs: 9 and 10, respectively. In some embodiments, the TGF-β inhibitor consists of the heavy chain and light chain set out in amino acid sequence of SEQ ID NOs: 9 and 10, respectively.

The TGF-β inhibitor can sometimes be administered at different doses. These doses can be effective at preventing, treating, or ameliorating a proliferative disease such as cancer or other solid tumors. In some embodiments, the TGF-β inhibitor is administered at a dose of about 20 mg/kg. In some embodiments, the TGF-β inhibitor is administered at a dose of about 30 mg/kg.

The subject being treated can vary in weight. In some embodiments, the subject can be about 50 to 90 kg. In some embodiments, the subject can be about 70 kg.

The TGF-β inhibitor can also be administered to a subject at a fixed dose. For example, in some embodiments, TGF-β inhibitor is administered to a subject at a dose of about 1200 mg to about 1600 mg. In other embodiments, the TGF-β inhibitor is administered at a dose of about 1400 mg. In some embodiments, the TGF-β inhibitor is administered at a dose of about 1900 mg to about 2300 mg. In some embodiments, the TGF-β inhibitor is administered at a dose of about 2100 mg.

The TGF-β inhibitor can be administered more than once at different intervals. For example, the TGF-β inhibitor can be administered once a week, once every two weeks, once every three weeks, or once every four weeks. In some embodiments, the TGF-β inhibitor is administered once a week. In some embodiments, the TGF-β inhibitor is administered once every two weeks. In some embodiments, the TGF-β inhibitor is administered once every three weeks. In some embodiments, the TGF-β inhibitor is administered once every four weeks.

In some embodiments, the TGF-β inhibitor is administered over a period of about 20 to about 40 minutes. In some embodiments, the TGF-β inhibitor is administered over a period of about 30 minutes.

The TGF-β inhibitor that is administered to the subject can be in any form such as a small chemical molecule, nucleic acid, or protein. For example, in some embodiments, the TGF-β inhibitor is an antibody. In some embodiments, the antibody is a monoclonal antibody. In some embodiments, the antibody is a monospecific antibody. In some embodiments, the TGF-β antibody is a multispecific antibody. If the TGF-β antibody is multispecific, the multispecific antibody is a bispecific antibody. In some embodiments, the multispecific antibody can be a trispecific antibody. In some embodiments, the multispecific antibody can bind specifically to four or more targets.

The methods of treatment described herein can also comprise administering to the subject a checkpoint inhibitor in combination with the TGF-β inhibitor. In some embodiments, the checkpoint inhibitor is a PD1 inhibitor. The PD1 inhibitor can be a small chemical molecule, nucleic acid, or protein. In some embodiments, the PD1 inhibitor is an anti-PD1 antibody. For example, the anti-PD1 antibody can be spartalizumab, nivolumab, pembrolizumab, pidilizumab, MEDI0680, REGN2810, TSR-042, PF-06801591, BGB-A317, BGB-108, INCSHR1210, AMP-224, or any combination thereof.

In some embodiments, the checkpoint inhibitor is an anti-PD1 antibody. For example, spartalizumab can be used as the anti-PD1 antibody. If spartalizumab is used, it can be administered as a flat dose. In some embodiments, spartalizumab is administered at about 300 mg or about 400 mg. In further embodiments, spartalizumab is administered once a week, once every two weeks, once every three weeks, or once every four weeks. For example, spartalizumab is administered once every three weeks. In some embodiments, spartalizumab is administered once every four weeks.

In some embodiments, the TGF-β inhibitor is administered intravenously. In some embodiments, the PD1 inhibitor is administered intravenously.

For combination treatments, in some embodiments, the TGF-β inhibitor is administered on the same day as the checkpoint inhibitor. In other embodiments, the TGF-β inhibitor is administered before the checkpoint inhibitor is administered. In additional embodiments, the TGF-β inhibitor is administered after the checkpoint inhibitor is administered. In some embodiments, the TGF-β inhibitor is administered at the same time as the checkpoint inhibitor. In some embodiments, the TGF-β inhibitor is administered for one or more dosing cycles until (partial or complete) remission. In some embodiments, the checkpoint inhibitor is administered for one or more dosing cycles until (partial or complete) remission.

The methods described can be used to treat proliferative disease. In some embodiments, the proliferative disease is a cancer (e.g., a solid tumor). In some embodiments, the cancer is a myelofibrosis, a myelodysplastic syndrome (e.g., low risk or high risk myelodysplastic syndrome), a leukemia, a lymphoma, a myeloma, a lung cancer, a gastrointestinal cancer, a skin cancer, an ovarian cancer, a mesothelioma, a bladder cancer, a soft tissue sarcoma, a bone cancer, a kidney cancer, a liver cancer, a cholangiocarcinoma, a sarcoma, a prostate cancer, a breast cancer (e.g., triple negative breast cancer), a colorectal cancer, a nasopharyngeal cancer, a duodenal cancer, an endometrial cancer, a pancreatic cancer, a head and neck cancer, an anal cancer, a gastro-esophageal cancer, a thyroid cancer, a cervical cancer, or a neuroendocrine tumor. The type of cancer that can be treated by the methods described can be a pancreatic cancer. In some embodiments, the pancreatic cancer is pancreatic ductal adenocarcinoma (PDAC). In some embodiments, the type of cancer that can be treated by the methods described can be a gastrointestinal cancer. In some embodiments, the gastrointestinal cancer is colorectal cancer. In some embodiments, the type of cancer that can be treated by the methods described can be myelofibrosis. In some embodiments, the type of cancer that can be treated by the methods described can be myelodysplastic syndrome. In some embodiments, the type of cancer that can be treated by the methods described can be breast cancer. For example, the breast cancer can be triple negative breast cancer. In some embodiments, the TGF-β inhibitor and/or checkpoint inhibitor is administered for one or more dosing cycles until remission.

In some embodiments, the method further comprises administering one or more anticancer therapies. In some embodiments, the anticancer therapy is chemotherapy, targeted therapies (e.g., antibodies or CAR T), radiation, any of the therapies described herein. In some embodiments, the anticancer therapy is a standard of care therapy.

Disclosed herein is a method of treating pancreatic ductal adenocarcinoma comprising administering to a subject in need thereof a TGF-β antibody at a dose of 2100 mg, once every two weeks, where the TGF-β antibody comprises the heavy chain and light chain set out in amino acid sequence of SEQ ID NOs: 9 and 10, respectively.

Disclosed herein is a method of treating pancreatic ductal adenocarcinoma comprising administering to a subject in need thereof a TGF-β antibody at a dose of 2100 mg, once every three weeks, where the TGF-β antibody comprises the heavy chain and light chain set out in amino acid sequence of SEQ ID NOs: 9 and 10, respectively.

Disclosed herein is a method of treating colorectal cancer comprising administering to a subject in need thereof a TGF-β antibody at a dose of 2100 mg, once every two weeks, wherein the TGF-β antibody comprises the heavy chain and light chain set out in amino acid sequence of SEQ ID NOs: 9 and 10, respectively.

Disclosed herein is a method of treating colorectal cancer comprising administering to a subject in need thereof a TGF-β antibody at a dose of 2100 mg, once every three weeks, wherein the TGF-β antibody comprises the heavy chain and light chain set out in amino acid sequence of SEQ ID NOs: 9 and 10, respectively.

Disclosed herein is a method of treating pancreatic ductal adenocarcinoma comprising administering to a subject in need thereof a TGF-β antibody at a dose of 1400 mg, once every two weeks, wherein the TGF-β antibody comprises the heavy chain and light chain set out in amino acid sequence of SEQ ID NOs: 9 and 10, respectively.

Disclosed herein is a method of treating colorectal cancer comprising administering to a subject in need thereof a TGF-β antibody at a dose of 1400 mg, once every three weeks, wherein the TGF-β antibody comprises the heavy chain and light chain set out in amino acid sequence of SEQ ID NOs: 9 and 10, respectively.

In some embodiments, the method can further comprise administering spartalizumab at a dose of about 300 mg once every three weeks. In some embodiments, the method can further comprise administering spartalizumab at a dose of about 400 mg once every four weeks.

Disclosed herein is a pharmaceutical composition comprising a TGF-β antibody and a pharmaceutically acceptable excipient, where the TGF-β antibody comprises the heavy chain and light chain set out in amino acid sequence of SEQ ID NOs: 9 and 10, respectively. In some embodiments, the TGF-β antibody is present at a concentration of 100 mg/mL. In some embodiments, the pharmaceutical composition comprises a histidine buffer at a concentration of 20 mM with a pH of 5.5. In some embodiments, the pharmaceutical composition comprises sucrose at a concentration of 220 mM. In some embodiments, the pharmaceutical composition comprises a surfactant or polysorbate 20 present at a concentration of 0.04% (w/w).

Other features, objects, and advantages of the invention will be apparent from the description and drawings, and from the claims.

### DESCRIPTION OF THE DRAWINGS

FIG. 1 shows the mean concentration-time profiles for each dose cohort of NIS793 for cycle 1

FIG. 2 shows the mean concentration-time profiles for each dose cohort of NIS793 for cycle 3.

### DETAILED DESCRIPTION

The immune system is responsible for the early detection and destruction of cancer cells. Cancer cells may escape immune surveillance through various mechanisms, such as reduced immune recognition, increased resistance to attack by immune cells or because of an immunosuppressive tumor microenvironment (Mittal et al 2014). Some cancers produce TGFβ, a potent immunosuppressive cytokine, which antagonizes cytotoxic lymphocytes and promotes the recruitment of inhibitory immune cells that favor tumor growth and progression (Wojtowicz-Praga-2003, Teicher 2007, Yang et al 2010).

TGFβ belongs to a large family of structurally-related cytokines including: bone morphogenetic proteins (BMPs), growth and differentiation factors, activins and inhibins. There are 3 isoforms of TGFβ ligand expressed in mammals, TGFβ1, 2, and 3. Under normal conditions, TGFβ maintains homeostasis and limits the growth of epithelial, endothelial, neuronal and hematopoietic cell lineages through the induction of anti-proliferative and apoptotic responses. Therefore it is believed that alterations of the TGFβ signaling pathway are involved in human diseases including cardio-vascular diseases, fibrosis, reproductive disorders, wound healing and cancers (Wakefield and Hill 2013).

NIS793 is a fully human IgG2, human/mouse cross-reactive, TGF-β-neutralizing antibody. NIS793 acts at the ligand-receptor level. Compared to fresolimumab which is a pan-TGFβ inhibitor that neutralizes all TGFβ isoforms, NIS793 more specifically antagonizes TGFβ1 and 2 and, to a lesser extent, TGFβ3.

To escape immune surveillance, cancer cells may additionally exploit immune checkpoint pathways that tightly regulate T-cell activation such as the PD-1/PD-L1 axis (Pardoll 2012). Thus, antagonism of TGFβ alone or in combination with immune checkpoint blockade may stimulate more potent anti-tumor immunity.

### Definitions

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

“About” and “approximately” means an acceptable degree of error for the quantity measured given the nature or precision of the measurements. Exemplary degrees of error are within 20%, typically, within 10%, and more typically, within 5% of a given value or range of values. In some embodiments, when a numerical number references the term “about” the number is intended to also include the exact value of the number. For example, “about 10” includes but is not limited to the value 10. It also includes 10±2, 10±1, or 10±0.5.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination can be administered together in a single composition or administered separately in different compositions. The administration of the therapeutic agents can be in any order. The first agent and the additional agents (e.g., second, third agents) can be administered via the same administration route or via different administration routes. In general, it is expected that additional therapeutic agents utilized in combination be utilized at levels that do not exceed the levels at which they are utilized individually. In some embodiments, the levels utilized in combination will be lower than those utilized individually.

In some embodiments, the additional therapeutic agent is administered at a therapeutic or lower-than therapeutic dose. In certain embodiments, the concentration of the second therapeutic agent that is required to achieve inhibition (e.g., growth inhibition) is lower when the second therapeutic agent is administered in combination with the first therapeutic agent (e.g., the anti-TGFβ antibody molecule) than when the second therapeutic agent (e.g., the anti-PD1 antibody molecule) is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor or a TGFβ inhibitor. For example, inhibition of an activity, e.g., a TGFβ, PD-1, or PD-L1 activity, of at least 5%, 10%, 20%, 30%, 40% or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells (e.g., proliferative disease). Cancer cells can spread locally or through the bloodstream and lymphatic system to other parts of the body. Examples of various cancers are described herein and include but are not limited to, solid tumors, e.g., lung cancer, breast cancer, prostate cancer, ovarian cancer, cervical cancer, skin cancer, pancreatic cancer, colorectal cancer, renal cancer, liver cancer, and brain cancer, and hematologic malignancies, e.g., lymphoma and leukemia, and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response.

Costimulatory molecules include, but are not limited to, an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

“Immune effector cell,” or “effector cell” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector” or “effector” “function” or “response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, can be cytolytic activity or helper activity including the secretion of cytokines.

As used herein, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments, the terms “treat,” “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment” and “treating” refer to the reduction or stabilization of tumor size or cancerous cell count.

The compositions, formulations, and methods of the present invention encompass polypeptides and nucleic acids having the sequences specified, or sequences substantially identical or similar thereto, e.g., sequences at least 80%, 81%, 82%, 83%, 84%, 85%, 86%, 87%, 88%, 89%, 90%, 91%, 92%, 93%, 94%, 95%, 96%, 97%, 98%, 99% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 81%, 82%, 83%, 84%, 85%, 86%, 87%, 88%, 89%, 90%, 91%, 92%, 93%, 94%, 95%, 96%, 97%, 98%, 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 81%, 82%, 83%, 84%, 85%, 86%, 87%, 88%, 89%, 90%, 91%, 92%, 93%, 94%, 95%, 96%, 97%, 98%, 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid (molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any one of the foregoing. As used herein the term “amino acid” includes both the D- or L-optical isomers and peptidomimetics.

A “conservative amino acid substitution” is one in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer can be linear or branched, it may comprise modified amino acids, and it can be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide can be either single-stranded or double-stranded, and if single-stranded can be the coding strand or non-coding (antisense) strand. A polynucleotide may comprise modified nucleotides, such as methylated nucleotides and nucleotide analogs. The sequence of nucleotides can be interrupted by non-nucleotide components. A polynucleotide can be further modified after polymerization, such as by conjugation with a labeling component. The nucleic acid can be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### TGF-β Inhibitors

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Transforming growth factor beta (also known as TGF-β, TGFβ, TGFb, or TGF-beta, used interchangeably herein) inhibitors (e.g., an anti-TGF-β antibody molecule) are described throughout and can be used in the methods described throughout.

In some embodiments, the TGF-β inhibitor is NIS793, fresolimumab, PF-06952229, or AVID200.

In some embodiments, the TGF-β inhibitor comprises NIS973, or a compound disclosed in International Application Publication No. WO 2012/167143. NIS793 is also known as XOMA 089 or XPA.42.089. NIS793 is a fully human monoclonal antibody that specifically binds and neutralizes TGF-beta 1 and 2 ligands.

The heavy chain CDR1, CDR2, and CDR3 of NIS793 has the amino sequence of: GGTFSSYAIS (SEQ ID NO: 1); GIIPIFGTANYAQKFQG (SEQ ID NO: 2); and GLWEVRALPSVY (SEQ ID NO: 3), respectively.

The light chain CDR1, CDR2, and CDR3 of NIS793 has the amino sequence of: GANDIGSKSVH (SEQ ID NO: 4); EDIIRPS (SEQ ID NO: 5); QVWDRDSDQYV (SEQ ID NO: 6), respectively.

The heavy chain variable region of NIS793 has the amino acid sequence of: QVQLVQSGAEVKKPGSSVKVSCKASGGTFSSYAISWVRQAPGQGLEWMGGIIPIFGTANYAQ KFQGRVTITADESTSTAYMELSSLRSEDTAVYYCARGLWEVRALPSVYWGQGTLVTVSS (SEQ ID NO: 7) (disclosed as SEQ ID NO: 6 in WO2012/167143). The light chain variable region of NIS793 has the amino acid sequence of: SYELTQPPSVSVAPGQTARITCGANDIGSKSVHWYQQKAGQAPVLVVSEDIIRPSGIPERISGS NSGNTATLTISRVEAGDEADYYCQVWDRDSDQYVFGTGTKVTVLG (SEQ ID NO: 8) (disclosed as SEQ ID NO: 8 in WO 2012/167143).

The heavy chain of NIS793 has the amino acid sequence of: QVQLVQSGAEVKKPGSSVKVSCKASGGTFSSYAISWVRQAPGQGLEWMGGIIPIFGTANYAQ KFQGRVTITADESTSTAYMELSSLRSEDTAVYYCARGLWEVRALPSVYWGQGTLVTVSSAST KGPSVFPLAPCSRSTSESTAALGCLVKDYFPEPVTVSWNSGALTSGVHTFPAVLQSSGLYSLS SVVTVTSSNFGTQTYTCNVDHKPSNTKVDKTVERKCCVECPPCPAPPVAGPSVFLFPPKPKDT LMISRTPEVTCVVVDVSHEDPEVQFNWYVDGMEVHNAKTKPREEQFNSTFRVVSVLTVVHQ DWLNGKEYKCKVSNKGLPAPIEKTISKTKGQPREPQVYTLPPSREEMTKNQVSLTCLVKGFY PSDIAVEWESNGQPENNYKTTPPMLDSDGSFFLYSKLTVDKSRWQQGNVFSCSVMHEALHN HYTQKSLSLSPGK (SEQ ID NO: 9). The light chain of NIS793 has the amino acid sequence of: SYELTQPPSVSVAPGQTARITCGANDIGSKSVHWYQQKAGQAPVLVVSEDIIRPSGIPERISGS NSGNTATLTISRVEAGDEADYYCQVWDRDSDQYVFGTGTKVTVLGQPKANPTVTLFPPSSEE LQANKATLVCLISDFYPGAVTVAWKADGSPVKAGVETTKPSKQSNNKYAASSYLSLTPEQW KSHRSYSCQVTHEGSTVEKTVAPTECS (SEQ ID NO: 10).

NIS793 binds with high affinity to the human TGF-β isoforms. Generally, NIS793 binds with high affinity to TGF-β1 and TGF-02, and to a lesser extent to TGF-03. In Biacore assays, the K_{D} of NIS793 on human TGF-β is 14.6 pM for TGF-β1, 67.3 pM for TGF-02, and 948 pM for TGF-β3. Given the high affinity binding to all three TGF-β isoforms, in certain embodiments, NIS793 is expected to bind to TGF-β1, 2 and 3 at a dose of NIS793 as described herein. NIS793 cross-reacts with rodent and cynomolgus monkey TGF-β and shows functional activity in vitro and in vivo, making rodent and cynomolgus monkey relevant species for toxicology studies.

In certain embodiments, a TGF-β inhibitor is used to treat a cancer (e.g., a pancreatic cancer such as PDAC or a gastrointestinal cancer such as colorectal cancer). In some embodiments, the TGF-β inhibitor is used combination with a checkpoint inhibitor (e.g., an inhibitor of PD1 described herein) is used to treat a cancer.

In some embodiments, the TGF-β inhibitor (e.g., NIS793) is administered at a dose of greater than 15 mg/kg. For example, the TGF-β inhibitor is administered at a dose of between 15.1 mg/kg and about 50 mg/kg. In some embodiments, the TGF-β inhibitor is administered at a dose of between about 16 mg/kg and about 50 mg/kg. In some embodiments, the TGF-β inhibitor is administered at a dose of between 16 mg/kg and about 50 mg/kg. In some embodiments, the TGF-β inhibitor is administered at a dose of between about 20 mg/kg and about 40 mg/kg. In some embodiments, the TGF-β inhibitor is administered at a dose of between about 25 mg/kg and about 35 mg/kg. In some embodiments, the TGF-β inhibitor is administered at a dose of about 20 mg/kg. In some embodiments, the TGF-β inhibitor is administered at a dose of about 30 mg/kg.

In some embodiments, the TGF-β inhibitor (e.g., NIS793) is administered at a fixed dose. For example, in some embodiments the TGF-β inhibitor is administered at a dose of between about 1000 mg to about 1600 mg. In some embodiments, the TGF-β inhibitor is administered at a dose of between about 1100 mg to about 1500 mg. In some embodiments, the TGF-β inhibitor is administered at a dose of between about 1200 to about 1400 mg. In some embodiments, the TGF-β inhibitor is administered at a dose of between about 1300 mg to about 1400 mg. In some embodiments, the TGF-β inhibitor is administered at a dose of between about 1300 mg to about 1500 mg. In some embodiments, the TGF-β inhibitor is administered at a dose of between about 1300 mg to about 1600 mg. In some embodiments, the TGF-β inhibitor is administered at a dose of between about 1200 mg to about 1500 mg. In some embodiments, the TGF-β inhibitor is administered at a dose of between about 1200 mg to about 1600 mg. In some embodiments, the TGF-β inhibitor is administered at a dose of between about 1400 mg to about 1500 mg. In some embodiments, the TGF-inhibitor is administered at a dose of between about 1400 mg to about 1600 mg. In some embodiments, the TGF-β inhibitor is administered at a dose of between about 1100 mg to about 1600 mg. In some embodiments, the TGF-β inhibitor is administered at a dose of between 1100 mg to about 1400 mg. In some embodiments, the TGF-β inhibitor is administered at a dose of between about 1100 mg to about 1300 mg. In some embodiments, the TGF-β inhibitor is administered at a dose of between about 1100 mg to about 1200 mg. In some embodiments, the TGF-β inhibitor is administered at a dose of between about 1000 mg to about 1500 mg. In some embodiments, the TGF-inhibitor is administered at a dose of between about 1000 mg to about 1400 mg. In some embodiments, the TGF-β inhibitor is administered at a dose of between about 1000 mg to about 1300 mg. In some embodiments, the TGF-β inhibitor is administered at a dose of between about 1000 mg to about 1200 mg. In some embodiments, the TGF-β inhibitor is administered at a dose of between or about 1000 mg to about 1100 mg. In some embodiments, the TGF-β inhibitor is administered at a dose of about 1000 mg. In some embodiments, the TGF-β inhibitor is administered at a dose of about 1100 mg. In some embodiments, the TGF-β inhibitor is administered at a dose of about 1200 mg. In some embodiments, the TGF-β inhibitor is administered at a dose of about 1300 mg. In some embodiments, the TGF-β inhibitor is administered at a dose of about 1400 mg. In some embodiments, the TGF-β inhibitor is administered at a dose of about 1500 mg. In some embodiments, the TGF-β inhibitor is administered at a dose of about 1600 mg. In some embodiments, the TGF-β inhibitor is administered at a dose of between about 1200 mg to about 2100 mg.

In some embodiments, the TGF-β inhibitor is administered at a dose of between about 2000 mg to about 2500 mg. In some embodiments, the TGF-β inhibitor is administered at a dose of between about 2000 mg to about 2400 mg. In some embodiments, the TGF-β inhibitor is administered at a dose of between about 1900 mg to about 2300 mg. In some embodiments, the TGF-inhibitor is administered at a dose of between about 1900 mg to about 2200. In some embodiments, the TGF-β inhibitor is administered at a dose of between about 2000 mg to about 2100 mg. In some embodiments, the TGF-β inhibitor is administered at a dose of between about 2100 mg to about 2500 mg. In some embodiments, the TGF-β inhibitor is administered at a dose of between about 2100 to about 2400 mg. In some embodiments, the TGF-β inhibitor is administered at a dose of between about 2100 to about 2300 mg. In some embodiments, the TGF-β inhibitor is administered at a dose of between about 2100 to about 2200 mg. In some embodiments, the TGF-β inhibitor is administered at a dose of between about 2200 to about 2500 mg. In some embodiments, the TGF-β inhibitor is administered at a dose of between about 2200 to about 2400 mg. In some embodiments, the TGF-β inhibitor is administered at a dose of between about 2200 to about 2300 mg. In some embodiments, the TGF-β inhibitor is administered at a dose of between about 2300 mg to about 2500 mg. In some embodiments, the TGF-β inhibitor is administered at a dose of between about 2300 mg to about 2400 mg. In some embodiments, the TGF-β inhibitor is administered at a dose of between about 2400 mg to about 2500 mg. In some embodiments, the TGF-β inhibitor is administered at a dose of about 2000 mg. In some embodiments, the TGF-β inhibitor is administered at a dose of about 2100 mg. In some embodiments, the TGF-β inhibitor is administered at a dose of about 2200 mg. In some embodiments, the TGF-β inhibitor is administered at a dose of about 2300 mg. In some embodiments, the TGF-β inhibitor is administered at a dose of about 2400 mg. In some embodiments, the TGF-β inhibitor is administered at a dose of about 2500 mg.

In some embodiments, the TGF-β inhibitor is administered once a week, once every two weeks, once every three weeks, or once every four weeks. In some embodiments, the TGF-β inhibitor is administered once a week. In some embodiments, the TGF-β inhibitor is administered once every two weeks. In some embodiments, the TGF-β inhibitor is administered once every three weeks. In some embodiments, the TGF-β inhibitor is administered once four three weeks.

In some embodiments, the TGF-β inhibitor is administered intravenously.

In some embodiments, the TGF-β inhibitor is administered over a period of about 20 minutes to about 40 minutes. For example, the TGF-β inhibitor is administered over a period of about 30 minutes. In some embodiments, the TGF-β inhibitor is administered over a period of about an hour. In some embodiments, the TGF-β inhibitor is administered over a period of about two hours. In some embodiments, the TGF-β inhibitor is administered over a period of about three hours. In some embodiments, the TGF-β inhibitor is administered over a period of about four hours. In some embodiments, the TGF-β inhibitor is administered over a period of about five hours. In some embodiments, the TGF-β inhibitor is administered over a period of about six hours.

In some embodiments, the TGF-β inhibitor (e.g., NIS793) is administered at a dose between about 1300 mg to about 1500 mg (e.g., about 1400 mg), intravenously, once every two weeks. In some embodiments, the TGF-β inhibitor (e.g., NIS793) is administered at a dose between about 2000 mg to about 2200 mg (e.g., about 2100 mg), intravenously, once every two weeks. In some embodiments, the TGF-β inhibitor (e.g., NIS793) is administered at a dose between about 2000 mg to about 2200 mg (e.g., about 2100 mg), intravenously, once every three weeks.

In some embodiments, the TGF-β inhibitor (e.g., NIS793) is administered at a dose between about 1300 mg to about 1500 mg (e.g., about 1400 mg), intravenously, over a period of about 20 minutes to about 40 minutes (e.g., about 30 minutes), once every two weeks. In some embodiments, the TGF-β inhibitor (e.g., NIS793) is administered at a dose between about 2000 mg to about 2200 mg (e.g., about 2100 mg), intravenously, over a period of about 20 minutes to about 40 minutes (e.g., about 30 minutes), once every two weeks. In some embodiments, the TGF-β inhibitor (e.g., NIS793) is administered at a dose between about 2000 mg to about 2200 mg (e.g., about 2100 mg), intravenously, over a period of about 20 minutes to about 40 minutes (e.g., about 30 minutes), once every three weeks.

In some embodiments, the methods described herein can further comprises one or more other therapeutic agents, procedures or modalities. In some embodiments, the TGF-β inhibitor (e.g., NIS793) is administered in combination with a PD1 inhibitor (e.g., an anti-PD1 antibody molecule) and/or a PD-L inhibitor (PD-L1 and/or PD-L2). In one embodiment, the methods described herein can comprise administering an inhibitor of an inhibitory (or immune checkpoint) molecule PD-1, PD-L1, PD-L2, and/or TGFβ. In one embodiment, the inhibitor is an antibody or antibody fragment that binds to PD-1, PD-L1, PD-L2, and/or TGFβ.

Alternatively, or in combination with the aforesaid methods, the methods described herein can be administered or used with, one or more of: an immunomodulator (e.g., an activator of a costimulatory molecule or an inhibitor of an inhibitory molecule, e.g., an immune checkpoint molecule); a vaccine, e.g., a therapeutic cancer vaccine; or other forms of cellular immunotherapy.

In certain embodiments, the methods described herein is administered or used in with a modulator of a costimulatory molecule or an inhibitory molecule, e.g., a co-inhibitory ligand or receptor.

Other Exemplary TGF-β Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 11)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

The light chain of fresolimumab has the amino acid sequence of:

(SEQ ID NO: 12)
ETVLTQSPGTLSLSPGERATLSCRASQSLGSSYLAWYQQKPGQAPRLLI
YGASSRAPGIPDRFSGSGSGTDFTLTISRLEPEDFAVYYCQQYADSPIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

Fresolimumab is disclosed, e.g., in International Application Publication No. WO 2006/086469, and U.S. Pat. Nos. 8,383,780 and 8,591,901.

In some embodiments, the TGF-β inhibitor is PF-06952229. PF-06952229 is an inhibitor of TGF-βR1, preventing signaling through the receptor and TGF-βR1-mediated immunosuppression thereby enhancing the anti-tumor immune response. PF-06952229 is disclosed, e.g., in Yano et al. Immunology 2019; 157(3) 232-47.

In some embodiments, the TGF-β inhibitor is AVID200. AVID200 is a TGF-β receptor ectodomain-IgG Fc fusion protein, which selectively targets and neutralizes TGF-β isoforms 1 and 3. AVID200 is disclosed, e.g., in O'Connor-McCourt, M D et al. Can. Res. 2018; 78(13).

### PD-1 Inhibitors

PD-1 is a CD28/CTLA-4 family member expressed, e.g., on activated CD4+ and CD8+ T cells, Tregs, and B cells. It negatively regulates effector T cell signaling and function. PD-1 is induced on tumor-infiltrating T cells, and can result in functional exhaustion or dysfunction (Keir et al. (2008) Annu. Rev. Immunol. 26:677-704; Pardoll et al. (2012) Nat Rev Cancer 12(4):252-64). PD-1 delivers a coinhibitory signal upon binding to either of its two ligands, Programmed Death-Ligand 1 (PD-L1) or Programmed Death-Ligand 2 (PD-L2). PD-L1 is expressed on a number of cell types, including T cells, natural killer (NK) cells, macrophages, dendritic cells (DCs), B cells, epithelial cells, vascular endothelial cells, as well as many types of tumors. High expression of PD-L1 on murine and human tumors has been linked to poor clinical outcomes in a variety of cancers (Keir et al. (2008) Annu. Rev. Immunol. 26:677-704; Pardoll et al. (2012) Nat Rev Cancer 12(4):252-64). PD-L2 is expressed on dendritic cells, macrophages, and some tumors. Blockade of the PD-1 pathway has been pre-clinically and clinically validated for cancer immunotherapy. Both preclinical and clinical studies have demonstrated that anti-PD-1 blockade can restore activity of effector T cells and results in robust anti-tumor response. For example, blockade of PD-1 pathway can restore exhausted/dysfunctional effector T cell function (e.g., proliferation, IFN-γ secretion, or cytolytic function) and/or inhibit Treg cell function (Keir et al. (2008) Annu. Rev. Immunol. 26:677-704; Pardoll et al. (2012) Nat Rev Cancer 12(4):252-64). Blockade of the PD-1 pathway can be effected with an antibody, an antigen binding fragment thereof, an immunoadhesin, a fusion protein, or oligopeptide of PD-1, PD-L1 and/or PD-L2.

As used herein, the term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

In certain embodiments, the TGFβ inhibitors as described herein are administered in combination with a PD-1 inhibitor. In some embodiments, the PD-1 inhibitor is spartalizumab (PDR001, Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof”. In one embodiment, the anti-PD-1 inhibitor is spartalizumab, also known as PDR001.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 13). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 14, a VHCDR2 amino acid sequence of SEQ ID NO: 15, and a VHCDR3 amino acid sequence of SEQ ID NO: 16; and a light chain variable region (VL) comprising a VLCDR1 amino acid sequence of SEQ ID NO: 23, a VLCDR2 amino acid sequence of SEQ ID NO: 24, and a VLCDR3 amino acid sequence of SEQ ID NO: 25, each disclosed in Table 1.

In one embodiment, the antibody molecule comprises a VH comprising a VHCDR1 encoded by the nucleotide sequence of SEQ ID NO: 37, a VHCDR2 encoded by the nucleotide sequence of SEQ ID NO: 38, and a VHCDR3 encoded by the nucleotide sequence of SEQ ID NO: 39; and a VL comprising a VLCDR1 encoded by the nucleotide sequence of SEQ ID NO: 42, a VLCDR2 encoded by the nucleotide sequence of SEQ ID NO: 43, and a VLCDR3 encoded by the nucleotide sequence of SEQ ID NO: 44, each disclosed in Table 1.

In one embodiment, the anti-PD-1 antibody molecule comprises a VH comprising the amino acid sequence of SEQ ID NO: 19, or an amino acid sequence at least 85%, 86%, 87%, 88%, 89%, 90%, 91%, 92%, 93%, 94%, 95%, 96%, 97%, 98%, or 99% identical or higher to SEQ ID NO: 19. In one embodiment, the anti-PD-1 antibody molecule comprises a VL comprising the amino acid sequence of SEQ ID NO: 33, or an amino acid sequence at least 85%, 86%, 87%, 88%, 89%, 90%, 91%, 92%, 93%, 94%, 95%, 96%, 97%, 98%, or 99% identical or higher to SEQ ID NO: 33. In one embodiment, the anti-PD-1 antibody molecule comprises a VL comprising the amino acid sequence of SEQ ID NO: 29, or an amino acid sequence at least 85%, 86%, 87%, 88%, 89%, 90%, 91%, 92%, 93%, 94%, 95%, 96%, 97%, 98%, or 99% identical or higher to SEQ ID NO: 29. In one embodiment, the anti-PD-1 antibody molecule comprises a VH comprising the amino acid sequence of SEQ ID NO: 19 and a VL comprising the amino acid sequence of SEQ ID NO: 33. In one embodiment, the anti-PD-1 antibody molecule comprises a VH comprising the amino acid sequence of SEQ ID NO: 19 and a VL comprising the amino acid sequence of SEQ ID NO: 33.

In one embodiment, the antibody molecule comprises a VH encoded by the nucleotide sequence of SEQ ID NO: 20, or a nucleotide sequence at least 85%, 86%, 87%, 88%, 89%, 90%, 91%, 92%, 93%, 94%, 95%, 96%, 97%, 98%, or 99% identical or higher to SEQ ID NO: 20. In one embodiment, the antibody molecule comprises a VL encoded by the nucleotide sequence of SEQ ID NO: 34 or 30, or a nucleotide sequence at least 85%, 86%, 87%, 88%, 89%, 90%, 91%, 92%, 93%, 94%, 95%, 96%, 97%, 98%, or 99% identical or higher to SEQ ID NO: 34 or 30. In one embodiment, the antibody molecule comprises a VH encoded by the nucleotide sequence of SEQ ID NO: 20 and a VL encoded by the nucleotide sequence of SEQ ID NO: 34 or 30.

In one embodiment, the anti-PD-1 antibody molecule comprises a heavy chain comprising the amino acid sequence of SEQ ID NO: 21, or an amino acid sequence at least 85%, 90%, 95%, or 99% identical or higher to SEQ ID NO: 21. In one embodiment, the anti-PD-1 antibody molecule comprises a light chain comprising the amino acid sequence of SEQ ID NO: 35, or an amino acid sequence at least 85%, 86%, 87%, 88%, 89%, 90%, 91%, 92%, 93%, 94%, 95%, 96%, 97%, 98%, or 99% identical or higher to SEQ ID NO: 35. In one embodiment, the anti-PD-1 antibody molecule comprises a light chain comprising the amino acid sequence of SEQ ID NO: 31, or an amino acid sequence at least 85%, 86%, 87%, 88%, 89%, 90%, 91%, 92%, 93%, 94%, 95%, 96%, 97%, 98%, or 99% identical or higher to SEQ ID NO: 31. In one embodiment, the anti-PD-1 antibody molecule comprises a heavy chain comprising the amino acid sequence of SEQ ID NO: 21 and a light chain comprising the amino acid sequence of SEQ ID NO: 35. In one embodiment, the anti-PD-1 antibody molecule comprises a heavy chain comprising the amino acid sequence of SEQ ID NO: 21 and a light chain comprising the amino acid sequence of SEQ ID NO: 31.

In one embodiment, the antibody molecule comprises a heavy chain encoded by the nucleotide sequence of SEQ ID NO: 22, or a nucleotide sequence at least 85%, 86%, 87%, 88%, 89%, 90%, 91%, 92%, 93%, 94%, 95%, 96%, 97%, 98%, or 99% identical or higher to SEQ ID NO: 22. In one embodiment, the antibody molecule comprises a light chain encoded by the nucleotide sequence of SEQ ID NO: 36 or 32, or a nucleotide sequence at least 85%, 86%, 87%, 88%, 89%, 90%, 91%, 92%, 93%, 94%, 95%, 96%, 97%, 98%, or 99% identical or higher to SEQ ID NO: 36 or 32. In one embodiment, the antibody molecule comprises a heavy chain encoded by the nucleotide sequence of SEQ ID NO: 22 and a light chain encoded by the nucleotide sequence of SEQ ID NO: 36 or 32.

The antibody molecules described herein can be made by vectors, host cells, and methods described in US 2015/0210769.

In some embodiments, the PD-1 inhibitor (e.g., spartalizumab) is administered at a flat dose of between about 100 mg to about 600 mg. In some embodiments, the PD-1 inhibitor is administered at a dose of between about 100 mg to about 500 mg. In some embodiments, the PD-1 inhibitor is administered at a dose of between about 100 mg to about 400 mg. In some embodiments, the PD-1 inhibitor is administered at a dose of between about 100 mg to about 300 mg. In some embodiments, the PD-1 inhibitor is administered at a dose of between about 100 mg to about 200 mg. In some embodiments, the PD-1 inhibitor is administered at a dose of between about 200 mg to about 600 mg. In some embodiments, the PD-1 inhibitor is administered at a dose of between about 200 mg to about 500 mg. In some embodiments, the PD-1 inhibitor is administered at a dose of between about 200 mg to about 400 mg. In some embodiments, the PD-1 inhibitor is administered at a dose of between about 200 mg to about 300 mg. In some embodiments, the PD-1 inhibitor is administered at a dose of between about 300 mg to about 600 mg. In some embodiments, the PD-1 inhibitor is administered at a dose of between about 300 mg to about 500 mg. In some embodiments, the PD-1 inhibitor is administered at a dose of between about 300 mg to about 400 mg. In some embodiments, the PD-1 inhibitor is administered at a dose of between about 400 mg to about 600 mg. In some embodiments, the PD-1 inhibitor is administered at a dose of between about 400 mg to about 500 mg. In some embodiments, the PD-1 inhibitor is administered at a dose of between about 500 mg to about 600 mg.

In some embodiments, the PD-1 inhibitor (e.g., spartalizumab) is administered at a flat dose of about 100 mg. In some embodiments, the PD-1 inhibitor is administered at a dose of about 200 mg. In some embodiments, the PD-1 inhibitor is administered at a dose of about 300 mg. In some embodiments, the PD-1 inhibitor is administered at a dose of about 400 mg. In some embodiments, the PD-1 inhibitor is administered at a dose of about 500 mg. In some embodiments, the PD-1 inhibitor is administered at a dose of about 600 mg.

In some embodiments, the PD-1 inhibitor (e.g., spartalizumab) is administered once every four weeks. In some embodiments, the PD-1 inhibitor is administered once every three weeks. In some embodiments, the PD-1 inhibitor is administered once every two weeks. In some embodiments, the PD-1 inhibitor is administered once every week.

In some embodiments, the PD-1 inhibitor (e.g., spartalizumab) is administered intravenously.

In some embodiments, the PD-1 inhibitor (e.g., spartalizumab) is administered over a period of about 20 minutes to 40 minutes (e.g., about 30 minutes). In some embodiments, the PD-1 inhibitor is administered over a period of about 30 minutes. In some embodiments, the PD-1 inhibitor is administered over a period of about an hour. In some embodiments, the PD-1 inhibitor is administered over a period of about two hours. In some embodiments, the PD-1 inhibitor is administered over a period of about three hours. In some embodiments, the PD-1 inhibitor is administered over a period of about four hours. In some embodiments, the PD-1 inhibitor is administered over a period of about five hours. In some embodiments, the PD-1 inhibitor is administered over a period of about six hours.

In some embodiments, the PD-1 inhibitor (e.g., spartalizumab) is administered at a dose between about 300 mg to about 500 mg (e.g., about 400 mg), intravenously, once every four weeks. In some embodiments, the PD-1 inhibitor is administered at a dose between about 200 mg to about 400 mg (e.g., about 300 mg), intravenously, once every three weeks. In some embodiments, spartaliziumab is administered at a dose of 400 mg, once every four weeks. In some embodiments, spartalizumab is administered at a dose of 300 mg, once every three weeks.

In some embodiments, the PD-1 inhibitor (e.g., spartalizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., spartalizumab) is administered in combination with a TGF-β inhibitor (e.g., NIS793).

TABLE 1
Amino acid and nucleotide sequences of
exemplary anti-PD-1 antibody molecules

| | | |
|---|---|---|
| BAP049-Clone-B HC | | |
| SEQ ID NO: 14 (Kabat) | HCDR1 | TYWMH |
| SEQ ID NO: 15 (Kabat) | HCDR2 | TNIYPGTGGSNFDEKFKN |
| SEQ ID NO: 16 (Kabat) | HCDR3 | WTTGTGAY |
| SEQ ID NO: 17 | HCDR1 | GYTFTTY |
| (Chothia) | | |
| SEQ ID NO: 18 | HCDR2 | YPGTGG |
| (Chothia) | | |
| SEQ ID NO: 16 | HCDR3 | WTTGTGAY |
| ; (Chothia) | | |
| | | EVQLVQSGAEVKKPGESLRISCKGSGYTFTTYWMHWVRQA |
| SEQ ID NO: 19 | VH | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 20 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 21 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 22 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-B LC | | |
| SEQ ID NO: 23 (Kabat) | LCDR1 | KSSQSLLDSGNQKNFLT |
| SEQ ID NO: 24 (Kabat) | LCDR2 | WASTRES |
| SEQ ID NO: 25 (Kabat) | LCDR3 | QNDYSYPYT |
| SEQ ID NO: 26 | LCDR1 | SQSLLDSGNQKNF |
| (Chothia) | | |
| SEQ ID NO: 27 | LCDR2 | WAS |
| (Chothia) | | |
| SEQ ID NO: 28 | LCDR3 | DYSYPY |
| (Chothia) | | |
| SEQ ID NO: 29 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 30 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 31 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 32 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | DNACCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-E HC | | |
| SEQ ID NO: 14 (Kabat) | HCDR1 | TYWMH |
| SEQ ID NO: 15 (Kabat) | HCDR2 | NIYPGTGGSNFDEKFKN |
| SEQ ID NO: 16 (Kabat) | HCDR3 | WTTGTGAY |
| SEQ ID NO: 17 | HCDR1 | GYTFTTY |
| (Chothia) | | |
| SEQ ID NO: 18 | HCDR2 | YPGTGG |
| (Chothia) | | |
| SEQ ID NO: 16 | HCDR3 | WTTGTGAY |
| (Chothia) | | |
| SEQ ID NO: 19 | VH | EVQLVQSGAEVKKPGESLRISCKGSGYTFTTYWMHWVRQA |
| | DNA | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 20 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 21 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 22 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-E LC | | |
| SEQ ID NO: 23 (Kabat) | LCDR1 | KSSQSLLDSGNQKNFLT |
| SEQ ID NO: 24 (Kabat) | LCDR2 | WASTRES |
| SEQ ID NO: 25 (Kabat) | LCDR3 | QNDYSYPYT |
| SEQ ID NO: 26 | LCDR1 | SQSLLDSGNQKNF |
| (Chothia) | | |
| SEQ ID NO: 27 | LCDR2 | WAS |
| (Chothia) | | |
| SEQ ID NO: 28 | LCDR3 | DYSYPY |
| (Chothia) | | |
| SEQ ID NO: 33 | VL | EIVLTQSPATLSLSPGERATLSCKSSQSLLDSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 34 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 35 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 36 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-B HC | | |
| SEQ ID NO: 37 (Kabat) | HCDR1 | ACCTACTGGATGCAC |
| SEQ ID NO: 38 (Kabat) | HCDR2 | AATATCTACCCCGGCACCGGCGGCTCTAACTTCGACGAG |
| | | AAGTTTAAGAAT |
| SEQ ID NO: 39 (Kabat) | HCDR3 | TGGACTACCGGCACAGGCGCCTAC |
| SEQ ID NO: 40 | HCDR1 | GGCTACACCTTCACTACCTAC |
| (Chothia) | | |
| SEQ ID NO: 41 | HCDR2 | TACCCCGGCACCGGCGGC |
| (Chothia) | | |
| SEQ ID NO: 39) | HCDR3 | TGGACTACCGGCACAGGCGCCTAC |
| (Chothia) | | |
| BAP049-Clone-B LC | | |
| SEQ ID NO: 42 (Kabat) | LCDR1 | AAATCTAGTCAGTCACTGCTGGATAGCGGTAATCAGAAG |
| | | AACTTCCTGACC |
| SEQ ID NO: 43 (Kabat) | LCDR2 | TGGGCCTCTACTAGAGAATCA |
| SEQ ID NO: 44 (Kabat) | LCDR3 | CAGAACGACTATAGCTACCCCTACACC |
| SEQ ID NO: 45 | LCDR1 | AGTCAGTCACTGCTGGATAGCGGTAATCAGAAGAACTTC |
| (Chothia) | | |
| SEQ ID NO: 46 | LCDR2 | TGGGCCTCT |
| (Chothia) | | |
| SEQ ID NO: 47 | LCDR3 | GACTATAGCTACCCCTAC |
| (Chothia) | | |
| BAP049-Clone-E HC | | |
| SEQ ID NO: 37 (Kabat) | HCDR1 | ACCTACTGGATGCAC |
| SEQ ID NO: 38 (Kabat) | HCDR2 | AATATCTACCCCGGCACCGGCGGCTCTAACTTCGACGAG |
| | | AAGTTTAAGAAT |
| SEQ ID NO: 39 (Kabat) | HCDR3 | TGGACTACCGGCACAGGCGCCTAC |
| SEQ ID NO: 40 | HCDR1 | GGCTACACCTTCACTACCTAC |
| (Chothia) | | |
| SEQ ID NO: 41 | HCDR2 | TACCCCGGCACCGGCGGC |
| (Chothia) | | |
| SEQ ID NO: 39 | HCDR3 | TGGACTACCGGCACAGGCGCCTAC |
| (Chothia) | | |
| BAP049-Clone-E LC | | |
| SEQ ID NO: 42 (Kabat) | LCDR1 | AAATCTAGTCAGTCACTGCTGGATAGCGGTAATCAGAAG |
| | | AACTTCCTGACC |
| SEQ ID NO: 43 (Kabat) | LCDR2 | TGGGCCTCTACTAGAGAATCA |
| SEQ ID NO: 44 (Kabat) | LCDR3 | CAGAACGACTATAGCTACCCCTACACC |
| SEQ ID NO: 45 | LCDR1 | AGTCAGTCACTGCTGGATAGCGGTAATCAGAAGAACTTC |
| (Chothia) | | |
| SEQ ID NO: 46 | LCDR2 | TGGGCCTCT |
| (Chothia) | | |
| SEQ ID NO: 47 | LCDR3 | GACTATAGCTACCCCTAC |
| (Chothia) | | |

Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 2.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 2.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342).

TABLE 2
Amino acid sequences of other exemplary anti-PD-1 antibody molecules

| | | |
|---|---|---|
| Nivolumab | | |
| SEQ ID NO: 48 | Heavy | QVQLVESGGGVVQPGRSLRLDCKASGITFSNSGMHWVRQAPGKGLEWVAVIWYD |
| | chain | GSKRYYADSVKGRFTISRDNSKNTLFLQMNSLRAEDTAVYYCATNDDYWGQGTL |
| | | VTVSSASTKGPSVFPLAPCSRSTSESTAALGCLVKDYFPEPVTVSWNSGALTSG |
| | | VHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNKGLPSS |
| | | IEKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQK |
| | | SLSLSLGK |
| SEQ ID NO: 49 | Light | EIVLTQSPATLSLSPGERATLSCRASQSVSSYLAWYQQKPGQAPRLLIYDASNR |
| | chain | ATGIPARFSGSGSGTDFTLTISSLEPEDFAVYYCQQSSNWPRTFGQGTKVEIKR |
| | | TVAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSFNRGEC |
| Pembrolizumab | | |
| SEQ ID NO: 50 | Heavy | QVQLVQSGVEVKKPGASVKVSCKASGYTFTNYYMYWVRQAPGQGLEWMGGINPS |
| | chain | NGGTNFNEKFKNRVTLTTDSSTTTAYMELKSLQFDDTAVYYCARRDYRFDMGFD |
| | | YWGQGTTVTVSSASTKGPSVFPLAPCSRSTSESTAALGCLVKDYFPEPVTVSWN |
| | | SGALTSGVHTFPAVLQSSGLYSLSSVVTVPSSSLGTKTYTCNVDHKPSNTKVDK |
| | | RVESKYGPPCPPCPAPEFLGGPSVFLFPPKPKDTLMISRTPEVTCVVVDVSQED |
| | | PEVQFNWYVDGVEVHNAKTKPREEQFNSTYRVVSVLTVLHQDWLNGKEYKCKVS |
| | | NKGLPSSIEKTISKAKGQPREPQVYTLPPSQEEMTKNQVSLTCLVKGFYPSDIA |
| | | VEWESNGQPENNYKTTPPVLDSDGSFFLYSRLTVDKSRWQEGNVFSCSVMHEAL |
| | | HNHYTQKSLSLSLGK |
| SEQ ID NO: 51 | Light | EIVLTQSPATLSLSPGERATLSCRASKGVSTSGYSYLHWYQQKPGQAPRLLIYL |
| | chain | ASYLESGVPARFSGSGSGTDFTLTISSLEPEDFAVYYCQHSRDLPLTFGGGTKV |
| | | EIKRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFNRG |
| | | EC |
| Pidilizumab | | |
| SEQ ID NO: 52 | Heavy | QVQLVQSGSELKKPGASVKISCKASGYTFTNYGMNWVRQAPGQGLQWMGWINTD |
| | chain | SGESTYAEEFKGRFVFSLDTSVNTAYLQITSLTAEDTGMYFCVRVGYDALDYWG |
| | | QGTLVTVSSASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSNTKVDKRVE |
| | | PKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEAL |
| | | HNHYTQKSLSLSPGK |
| SEQ ID NO: 53 | Light | EIVLTQSPSSLSASVGDRVTITCSARSSVSYMHWFQQKPGKAPKLWIYRTSNLA |
| | chain | SGVPSRFSGSGSGTSYCLTINSLQPEDFATYYCQQRSSFPLTFGGGTKLEIKRT |
| | | VAAPSVFIFPPSDEQLKSGTASVVCLLNNFYPREAKVQWKVDNALQSGNSQESV |
| | | TEQDSKDSTYSLSSTLTLSKADYEKHKVYACEVTHQGLSSPVTKSFNRGEC |

### PD-L1 Inhibitors

In certain embodiments, the method described further includes administering a PD-L1 inhibitor. In some embodiments, the PD-L1 inhibitor is FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof.”

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 3 (e.g., from the heavy and light chain variable region sequences of BAP058-Clone 0 or BAP058-Clone N disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTSYWMY (SEQ ID NO: 100). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-L1 antibody molecule comprises a heavy chain variable region (VH) comprising a VHCDR1 amino acid sequence of SEQ ID NO: 54, a VHCDR2 amino acid sequence of SEQ ID NO: 55, and a VHCDR3 amino acid sequence of SEQ ID NO: 56; and a light chain variable region (VL) comprising a VLCDR1 amino acid sequence of SEQ ID NO: 62, a VLCDR2 amino acid sequence of SEQ ID NO: 63, and a VLCDR3 amino acid sequence of SEQ ID NO: 64, each disclosed in Table 3.

In one embodiment, the anti-PD-L1 antibody molecule comprises a VH comprising a VHCDR1 encoded by the nucleotide sequence of SEQ ID NO: 81, a VHCDR2 encoded by the nucleotide sequence of SEQ ID NO: 82, and a VHCDR3 encoded by the nucleotide sequence of SEQ ID NO: 83; and a VL comprising a VLCDR1 encoded by the nucleotide sequence of SEQ ID NO: 86, a VLCDR2 encoded by the nucleotide sequence of SEQ ID NO: 87, and a VLCDR3 encoded by the nucleotide sequence of SEQ ID NO: 88, each disclosed in Table 3.

In one embodiment, the anti-PD-L1 antibody molecule comprises a VH comprising the amino acid sequence of SEQ ID NO: 59, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 59. In one embodiment, the anti-PD-L1 antibody molecule comprises a VL comprising the amino acid sequence of SEQ ID NO: 69, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 69. In one embodiment, the anti-PD-L1 antibody molecule comprises a VH comprising the amino acid sequence of SEQ ID NO: 73, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 73. In one embodiment, the anti-PD-L1 antibody molecule comprises a VL comprising the amino acid sequence of SEQ ID NO: 77, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 77. In one embodiment, the anti-PD-L1 antibody molecule comprises a VH comprising the amino acid sequence of SEQ ID NO: 59 and a VL comprising the amino acid sequence of SEQ ID NO: 69. In one embodiment, the anti-PD-L1 antibody molecule comprises a VH comprising the amino acid sequence of SEQ ID NO: 73 and a VL comprising the amino acid sequence of SEQ ID NO: 77.

In one embodiment, the antibody molecule comprises a VH encoded by the nucleotide sequence of SEQ ID NO: 60, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 60. In one embodiment, the antibody molecule comprises a VL encoded by the nucleotide sequence of SEQ ID NO: 70, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 70. In one embodiment, the antibody molecule comprises a VH encoded by the nucleotide sequence of SEQ ID NO: 74, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 74. In one embodiment, the antibody molecule comprises a VL encoded by the nucleotide sequence of SEQ ID NO: 78, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 78. In one embodiment, the antibody molecule comprises a VH encoded by the nucleotide sequence of SEQ ID NO: 60 and a VL encoded by the nucleotide sequence of SEQ ID NO: 70. In one embodiment, the antibody molecule comprises a VH encoded by the nucleotide sequence of SEQ ID NO: 72 and a VL encoded by the nucleotide sequence of SEQ ID NO: 78.

In one embodiment, the anti-PD-L1 antibody molecule comprises a heavy chain comprising the amino acid sequence of SEQ ID NO: 61, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 61. In one embodiment, the anti-PD-L1 antibody molecule comprises a light chain comprising the amino acid sequence of SEQ ID NO: 71, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 71. In one embodiment, the anti-PD-L1 antibody molecule comprises a heavy chain comprising the amino acid sequence of SEQ ID NO: 75, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 75. In one embodiment, the anti-PD-L1 antibody molecule comprises a light chain comprising the amino acid sequence of SEQ ID NO: 79, or an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 79. In one embodiment, the anti-PD-L1 antibody molecule comprises a heavy chain comprising the amino acid sequence of SEQ ID NO: 61 and a light chain comprising the amino acid sequence of SEQ ID NO: 71. In one embodiment, the anti-PD-L1 antibody molecule comprises a heavy chain comprising the amino acid sequence of SEQ ID NO: 75 and a light chain comprising the amino acid sequence of SEQ ID NO: 79.

In one embodiment, the antibody molecule comprises a heavy chain encoded by the nucleotide sequence of SEQ ID NO: 68, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 68. In one embodiment, the antibody molecule comprises a light chain encoded by the nucleotide sequence of SEQ ID NO: 72, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 72. In one embodiment, the antibody molecule comprises a heavy chain encoded by the nucleotide sequence of SEQ ID NO: 76, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 76. In one embodiment, the antibody molecule comprises a light chain encoded by the nucleotide sequence of SEQ ID NO: 80, or a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical or higher to SEQ ID NO: 80. In one embodiment, the antibody molecule comprises a heavy chain encoded by the nucleotide sequence of SEQ ID NO: 68 and a light chain encoded by the nucleotide sequence of SEQ ID NO: 72. In one embodiment, the antibody molecule comprises a heavy chain encoded by the nucleotide sequence of SEQ ID NO: 76 and a light chain encoded by the nucleotide sequence of SEQ ID NO: 80.

The antibody molecules described herein can be made by vectors, host cells, and methods described in US 2016/0108123.

TABLE 3
Amino acid and nucleotide sequences of
exemplary anti-PD-Ll antibody molecules

| | | |
|---|---|---|
| BAP058-Clone O HC | | |
| SEQ ID NO: 54 (Kabat) | HCDR1 | SYWMY |
| SEQ ID NO: 55 (Kabat) | HCDR2 | RIDPNSGSTKYNEKFKN |
| SEQ ID NO: 56 (Kabat) | HCDR3 | DYRKGLYAMDY |
| SEQ ID NO: 57 | HCDR1 | GYTFTSY |
| (Chothia) | | |
| SEQ ID NO: 58 | HCDR2 | DPNSGS |
| (Chothia) | | |
| SEQ ID NO: 56 | HCDR3 | DYRKGLYAMDY |
| (Chothia) | | |
| SEQ ID NO: 59 | VH | EVQLVQSGAEVKKPGATVKISCKVSGYTFTSYWMYWVRQ |
| | | ARGQRLEWIGRIDPNSGSTKYNEKFKNRFTISRDNSKNTLY |
| | | LQMNSLRAEDTAVYYCARDYRKGLYAMDYWGQGTTVTV |
| | | SS |
| SEQ ID NO: 60 | DNAVH | GAAGTGCAGCTGGTGCAGTCAGGCGCCGAAGTGAAGAA |
| | | ACCCGGCGCTACCGTGAAGATTAGCTGTAAAGTCTCAGG |
| | | CTACACCTTCACTAGCTACTGGATGTACTGGGTCCGACA |
| | | GGCTAGAGGGCAAAGACTGGAGTGGATCGGTAGAATCG |
| | | ACCCTAATAGCGGCTCTACTAAGTATAACGAGAAGTTTA |
| | | AGAATAGGTTCACTATTAGTAGGGATAACTCTAAGAACA |
| | | CCCTGTACCTGCAGATGAATAGCCTGAGAGCCGAGGAC |
| | | ACCGCCGTCTACTACTGCGCTAGAGACTATAGAAAGGGC |
| | | CTGTACGCTATGGACTACTGGGGTCAAGGCACTACCGTG |
| | | ACCGTGTCTTCA |
| SEQ ID NO: 61 | Heavy | EVQLVQSGAEVKKPGATVKISCKVSGYTFTSYWMYWVRQ |
| | chain | ARGQRLEWIGRIDPNSGSTKYNEKFKNRFTISRDNSKNTLY |
| | | LQMNSLRAEDTAVYYCARDYRKGLYAMDYWGQGTTVTV |
| | | SSASTKGPSVFPLAPCSRSTSESTAALGCLVKDYFPEPVTVS |
| | | WNSGALTSGVHTFPAVLQSSGLYSLSSVVTVPSSSLGTKTY |
| | | TCNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 68 | DNA | GAAGTGCAGCTGGTGCAGTCAGGCGCCGAAGTGAAGAA |
| | heavy | ACCCGGCGCTACCGTGAAGATTAGCTGTAAAGTCTCAGG |
| | chain | CTACACCTTCACTAGCTACTGGATGTACTGGGTCCGACA |
| | | GGCTAGAGGGCAAAGACTGGAGTGGATCGGTAGAATCG |
| | | ACCCTAATAGCGGCTCTACTAAGTATAACGAGAAGTTTA |
| | | AGAATAGGTTCACTATTAGTAGGGATAACTCTAAGAACA |
| | | CCCTGTACCTGCAGATGAATAGCCTGAGAGCCGAGGAC |
| | | ACCGCCGTCTACTACTGCGCTAGAGACTATAGAAAGGGC |
| | | CTGTACGCTATGGACTACTGGGGTCAAGGCACTACCGTG |
| | | ACCGTGTCTTCAGCTAGCACTAAGGGCCCGTCCGTGTTC |
| | | CCCCTGGCACCTTGTAGCCGGAGCACTAGCGAATCCACC |
| | | GCTGCCCTCGGCTGCCTGGTCAAGGATTACTTCCCGGAG |
| | | CCCGTGACCGTGTCCTGGAACAGCGGAGCCCTGACCTCC |
| | | GGAGTGCACACCTTCCCCGCTGTGCTGCAGAGCTCCGGG |
| | | CTGTACTCGCTGTCGTCGGTGGTCACGGTGCCTTCATCTA |
| | | GCCTGGGTACCAAGACCTACACTTGCAACGTGGACCACA |
| | | AGCCTTCCAACACTAAGGTGGACAAGCGCGTCGAATCG |
| | | AAGTACGGCCCACCGTGCCCGCCTTGTCCCGCGCCGGAG |
| | | TTCCTCGGCGGTCCCTCGGTCTTTCTGTTCCCACCGAAGC |
| | | CCAAGGACACTTTGATGATTTCCCGCACCCCTGAAGTGA |
| | | CATGCGTGGTCGTGGACGTGTCACAGGAAGATCCGGAG |
| | | GTGCAGTTCAATTGGTACGTGGATGGCGTCGAGGTGCAC |
| | | AACGCCAAAACCAAGCCGAGGGAGGAGCAGTTCAACTC |
| | | CACTTACCGCGTCGTGTCCGTGCTGACGGTGCTGCATCA |
| | | GGACTGGCTGAACGGGAAGGAGTACAAGTGCAAAGTGT |
| | | CCAACAAGGGACTTCCTAGCTCAATCGAAAAGACCATCT |
| | | CGAAAGCCAAGGGACAGCCCCGGGAACCCCAAGTGTAT |
| | | ACCCTGCCACCGAGCCAGGAAGAAATGACTAAGAACCA |
| | | AGTCTCATTGACTTGCCTTGTGAAGGGCTTCTACCCATCG |
| | | GATATCGCCGTGGAATGGGAGTCCAACGGCCAGCCGGA |
| | | AAACAACTACAAGACCACCCCTCCGGTGCTGGACTCAGA |
| | | CGGATCCTTCTTCCTCTACTCGCGGCTGACCGTGGATAA |
| | | GAGCAGATGGCAGGAGGGAAATGTGTTCAGCTGTTCTGT |
| | | GATGCATGAAGCCCTGCACAACCACTACACTCAGAAGTC |
| | | CCTGTCCCTCTCCCTGGGA |
| BAP058-Clone O LC | | |
| SEQ ID NO: 62 (Kabat) | LCDR1 | KASQDVGTAVA |
| SEQ ID NO: 63 (Kabat) | LCDR2 | WASTRHT |
| SEQ ID NO: 64 (Kabat) | LCDR3 | QQYNSYPLT |
| SEQ ID NO: 65 | LCDR1 | SQDVGTA |
| (Chothia) | | |
| SEQ ID NO: 66 | LCDR2 | WAS |
| (Chothia) | | |
| SEQ ID NO: 67 | LCDR3 | YNSYPL |
| (Chothia) | | |
| SEQ ID NO: 69 | VL | AIQLTQSPSSLSASVGDRVTITCKASQDVGTAVAWYLQKPG |
| | | QSPQLLIYWASTRHTGVPSRFSGSGSGTDFTFTISSLEAEDA |
| | | ATYYCQQYNSYPLTFGQGTKVEIK |
| SEQ ID NO: 70 | DNAVL | GCTATTCAGCTGACTCAGTCACCTAGTAGCCTGAGCGCT |
| | | AGTGTGGGCGATAGAGTGACTATCACCTGTAAAGCCTCT |
| | | CAGGACGTGGGCACCGCCGTGGCCTGGTATCTGCAGAA |
| | | GCCTGGTCAATCACCTCAGCTGCTGATCTACTGGGCCTC |
| | | TACTAGACACACCGGCGTGCCCTCTAGGTTTAGCGGTAG |
| | | CGGTAGTGGCACCGACTTCACCTTCACTATCTCTTCACTG |
| | | GAAGCCGAGGACGCCGCTACCTACTACTGTCAGCAGTAT |
| | | AATAGCTACCCCCTGACCTTCGGTCAAGGCACTAAGGTC |
| | | GAGATTAAG |
| SEQ ID NO: 71 | Light | AIQLTQSPSSLSASVGDRVTITCKASQDVGTAVAWYLQKPG |
| | chain | QSPQLLIYWASTRHTGVPSRFSGSGSGTDFTFTISSLEAEDA |
| | | ATYYCQQYNSYPLTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSSP |
| | | VTKSFNRGEC |
| SEQ ID NO: 72 | DNA light | GCTATTCAGCTGACTCAGTCACCTAGTAGCCTGAGCGCT |
| | chain | AGTGTGGGCGATAGAGTGACTATCACCTGTAAAGCCTCT |
| | | CAGGACGTGGGCACCGCCGTGGCCTGGTATCTGCAGAA |
| | | GCCTGGTCAATCACCTCAGCTGCTGATCTACTGGGCCTC |
| | | TACTAGACACACCGGCGTGCCCTCTAGGTTTAGCGGTAG |
| | | CGGTAGTGGCACCGACTTCACCTTCACTATCTCTTCACTG |
| | | GAAGCCGAGGACGCCGCTACCTACTACTGTCAGCAGTAT |
| | | AATAGCTACCCCCTGACCTTCGGTCAAGGCACTAAGGTC |
| | | GAGATTAAGCGTACGGTGGCCGCTCCCAGCGTGTTCATC |
| | | TTCCCCCCCAGCGACGAGCAGCTGAAGAGCGGCACCGC |
| | | CAGCGTGGTGTGCCTGCTGAACAACTTCTACCCCCGGGA |
| | | GGCCAAGGTGCAGTGGAAGGTGGACAACGCCCTGCAGA |
| | | GCGGCAACAGCCAGGAGAGCGTCACCGAGCAGGACAGC |
| | | AAGGACTCCACCTACAGCCTGAGCAGCACCCTGACCCTG |
| | | AGCAAGGCCGACTACGAGAAGCATAAGGTGTACGCCTG |
| | | CGAGGTGACCCACCAGGGCCTGTCCAGCCCCGTGACCA |
| | | AGAGCTTCAACAGGGGCGAGTGC |
| BAP058-Clone N HC | | |
| SEQ ID NO: 54 (Kabat) | HCDR1 | SYWMY |
| SEQ ID NO: 55 (Kabat) | HCDR2 | RIDPNSGSTKYNEKFKN |
| SEQ ID NO: 56 (Kabat) | HCDR3 | DYRKGLYAMDY |
| SEQ ID NO: 57 | HCDR1 | GYTFTSY |
| (Chothia) | | |
| SEQ ID NO: 58 | HCDR2 | DPNSGS |
| (Chothia) | | |
| SEQ ID NO: 56 | HCDR3 | DYRKGLYAMDY |
| (Chothia) | | |
| SEQ ID NO: 73 | VH | EVQLVQSGAEVKKPGATVKISCKVSGYTFTSYWMYWVRQ |
| | | ATGQGLEWMGRIDPNSGSTKYNEKFKNRVTITADKSTSTA |
| | | YMELSSLRSEDTAVYYCARDYRKGLYAMDYWGQGTTVT |
| | | VSS |
| SEQ ID NO: 74 | DNAVH | GAAGTGCAGCTGGTGCAGTCAGGCGCCGAAGTGAAGAA |
| | | ACCCGGCGCTACCGTGAAGATTAGCTGTAAAGTCTCAGG |
| | | CTACACCTTCACTAGCTACTGGATGTACTGGGTCCGACA |
| | | GGCTACCGGTCAAGGCCTGGAGTGGATGGGTAGAATCG |
| | | ACCCTAATAGCGGCTCTACTAAGTATAACGAGAAGTTTA |
| | | AGAATAGAGTGACTATCACCGCCGATAAGTCTACTAGCA |
| | | CCGCCTATATGGAACTGTCTAGCCTGAGATCAGAGGACA |
| | | CCGCCGTCTACTACTGCGCTAGAGACTATAGAAAGGGCC |
| | | TGTACGCTATGGACTACTGGGGTCAAGGCACTACCGTGA |
| | | CCGTGTCTTCA |
| SEQ ID NO: 75 | Heavy | EVQLVQSGAEVKKPGATVKISCKVSGYTFTSYWMYWVRQ |
| | chain | ATGQGLEWMGRIDPNSGSTKYNEKFKNRVTITADKSTSTA |
| | | YMELSSLRSEDTAVYYCARDYRKGLYAMDYWGQGTTVT |
| | | VSSASTKGPSVFPLAPCSRSTSESTAALGCLVKDYFPEPVTV |
| | | SWNSGALTSGVHTFPAVLQSSGLYSLSSVVTVPSSSLGTKT |
| | | YTCNVDHKPSNTKVDKRVESKYGPPCPPCPAPEFLGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYK |
| | | CKVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| SEQ ID NO: 76 | DNA | GAAGTGCAGCTGGTGCAGTCAGGCGCCGAAGTGAAGAA |
| | heavy | ACCCGGCGCTACCGTGAAGATTAGCTGTAAAGTCTCAGG |
| | chain | CTACACCTTCACTAGCTACTGGATGTACTGGGTCCGACA |
| | | GGCTACCGGTCAAGGCCTGGAGTGGATGGGTAGAATCG |
| | | ACCCTAATAGCGGCTCTACTAAGTATAACGAGAAGTTTA |
| | | AGAATAGAGTGACTATCACCGCCGATAAGTCTACTAGCA |
| | | CCGCCTATATGGAACTGTCTAGCCTGAGATCAGAGGACA |
| | | CCGCCGTCTACTACTGCGCTAGAGACTATAGAAAGGGCC |
| | | TGTACGCTATGGACTACTGGGGTCAAGGCACTACCGTGA |
| | | CCGTGTCTTCAGCTAGCACTAAGGGCCCGTCCGTGTTCC |
| | | CCCTGGCACCTTGTAGCCGGAGCACTAGCGAATCCACCG |
| | | CTGCCCTCGGCTGCCTGGTCAAGGATTACTTCCCGGAGC |
| | | CCGTGACCGTGTCCTGGAACAGCGGAGCCCTGACCTCCG |
| | | GAGTGCACACCTTCCCCGCTGTGCTGCAGAGCTCCGGGC |
| | | TGTACTCGCTGTCGTCGGTGGTCACGGTGCCTTCATCTAG |
| | | CCTGGGTACCAAGACCTACACTTGCAACGTGGACCACAA |
| | | GCCTTCCAACACTAAGGTGGACAAGCGCGTCGAATCGA |
| | | AGTACGGCCCACCGTGCCCGCCTTGTCCCGCGCCGGAGT |
| | | TCCTCGGCGGTCCCTCGGTCTTTCTGTTCCCACCGAAGCC |
| | | CAAGGACACTTTGATGATTTCCCGCACCCCTGAAGTGAC |
| | | ATGCGTGGTCGTGGACGTGTCACAGGAAGATCCGGAGG |
| | | TGCAGTTCAATTGGTACGTGGATGGCGTCGAGGTGCACA |
| | | ACGCCAAAACCAAGCCGAGGGAGGAGCAGTTCAACTCC |
| | | ACTTACCGCGTCGTGTCCGTGCTGACGGTGCTGCATCAG |
| | | GACTGGCTGAACGGGAAGGAGTACAAGTGCAAAGTGTC |
| | | CAACAAGGGACTTCCTAGCTCAATCGAAAAGACCATCTC |
| | | GAAAGCCAAGGGACAGCCCCGGGAACCCCAAGTGTATA |
| | | CCCTGCCACCGAGCCAGGAAGAAATGACTAAGAACCAA |
| | | GTCTCATTGACTTGCCTTGTGAAGGGCTTCTACCCATCGG |
| | | ATATCGCCGTGGAATGGGAGTCCAACGGCCAGCCGGAA |
| | | AACAACTACAAGACCACCCCTCCGGTGCTGGACTCAGAC |
| | | GGATCCTTCTTCCTCTACTCGCGGCTGACCGTGGATAAG |
| | | AGCAGATGGCAGGAGGGAAATGTGTTCAGCTGTTCTGTG |
| | | ATGCATGAAGCCCTGCACAACCACTACACTCAGAAGTCC |
| | | CTGTCCCTCTCCCTGGGA |
| BAP058-Clone N LC | | |
| SEQ ID NO: 62 (Kabat) | LCDR1 | KASQDVGTAVA |
| SEQ ID NO: 63 (Kabat) | LCDR2 | WASTRHT |
| SEQ ID NO: 64 (Kabat) | LCDR3 | QQYNSYPLT |
| SEQ ID NO: 65 | LCDR1 | SQDVGTA |
| (Chothia) | | |
| SEQ ID NO: 66 | LCDR2 | WAS |
| (Chothia) | | |
| SEQ ID NO: 67 | LCDR3 | YNSYPL |
| (Chothia) | | |
| SEQ ID NO: 77 | VL | DVVMTQSPLSLPVTLGQPASISCKASQDVGTAVAWYQQKP |
| | | GQAPRLLIYWASTRHTGVPSRFSGSGSGTEFTLTISSLQPDD |
| | | FATYYCQQYNSYPLTFGQGTKVEIK |
| SEQ ID NO: 78 | DNAVL | GACGTCGTGATGACTCAGTCACCCCTGAGCCTGCCCGTG |
| | | ACCCTGGGGCAGCCCGCCTCTATTAGCTGTAAAGCCTCT |
| | | CAGGACGTGGGCACCGCCGTGGCCTGGTATCAGCAGAA |
| | | GCCAGGGCAAGCCCCTAGACTGCTGATCTACTGGGCCTC |
| | | TACTAGACACACCGGCGTGCCCTCTAGGTTTAGCGGTAG |
| | | CGGTAGTGGCACCGAGTTCACCCTGACTATCTCTTCACT |
| | | GCAGCCCGACGACTTCGCTACCTACTACTGTCAGCAGTA |
| | | TAATAGCTACCCCCTGACCTTCGGTCAAGGCACTAAGGT |
| | | CGAGATTAAG |
| SEQ ID NO: 79 | Light | DVVMTQSPLSLPVTLGQPASISCKASQDVGTAVAWYQQKP |
| | chain | GQAPRLLIYWASTRHTGVPSRFSGSGSGTEFTLTISSLQPDD |
| | | FATYYCQQYNSYPLTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 80 | DNA light | GACGTCGTGATGACTCAGTCACCCCTGAGCCTGCCCGTG |
| | chain | ACCCTGGGGCAGCCCGCCTCTATTAGCTGTAAAGCCTCT |
| | | CAGGACGTGGGCACCGCCGTGGCCTGGTATCAGCAGAA |
| | | GCCAGGGCAAGCCCCTAGACTGCTGATCTACTGGGCCTC |
| | | TACTAGACACACCGGCGTGCCCTCTAGGTTTAGCGGTAG |
| | | CGGTAGTGGCACCGAGTTCACCCTGACTATCTCTTCACT |
| | | GCAGCCCGACGACTTCGCTACCTACTACTGTCAGCAGTA |
| | | TAATAGCTACCCCCTGACCTTCGGTCAAGGCACTAAGGT |
| | | CGAGATTAAGCGTACGGTGGCCGCTCCCAGCGTGTTCAT |
| | | CTTCCCCCCCAGCGACGAGCAGCTGAAGAGCGGCACCG |
| | | CCAGCGTGGTGTGCCTGCTGAACAACTTCTACCCCCGGG |
| | | AGGCCAAGGTGCAGTGGAAGGTGGACAACGCCCTGCAG |
| | | AGCGGCAACAGCCAGGAGAGCGTCACCGAGCAGGACAG |
| | | CAAGGACTCCACCTACAGCCTGAGCAGCACCCTGACCCT |
| | | GAGCAAGGCCGACTACGAGAAGCATAAGGTGTACGCCT |
| | | GCGAGGTGACCCACCAGGGCCTGTCCAGCCCCGTGACC |
| | | AAGAGCTTCAACAGGGGCGAGTGC |
| BAP058-Clone O HC | | |
| SEQ ID NO: 81 (Kabat) | HCDR1 | AGCTACTGGATGTAC |
| SEQ ID NO: 82 (Kabat) | HCDR2 | AGAATCGACCCTAATAGCGGCTCTACTAAGTATAACGAG |
| | | AAGTTTAAGAAT |
| SEQ ID NO: 83 (Kabat) | HCDR3 | GACTATAGAAAGGGCCTGTACGCTATGGACTAC |
| SEQ ID NO: 84 | HCDR1 | GGCTACACCTTCACTAGCTAC |
| (Chothia) | | |
| SEQ ID NO: 85 | HCDR2 | GACCCTAATAGCGGCTCT |
| (Chothia) | | |
| SEQ ID NO: 83 | HCDR3 | GACTATAGAAAGGGCCTGTACGCTATGGACTAC |
| (Chothia) | | |
| BAP058-Clone O LC | | |
| SEQ ID NO: 86 (Kabat) | LCDR1 | AAAGCCTCTCAGGACGTGGGCACCGCCGTGGCC |
| SEQ ID NO: 87 (Kabat) | LCDR2 | TGGGCCTCTACTAGACACACC |
| SEQ ID NO: 88 (Kabat) | LCDR3 | CAGCAGTATAATAGCTACCCCCTGACC |
| SEQ ID NO: 89 | LCDR1 | TCTCAGGACGTGGGCACCGCC |
| (Chothia) | | |
| SEQ ID NO: 90 | LCDR2 | TGGGCCTCT |
| (Chothia) | | |
| SEQ ID NO: 91 | LCDR3 | TATAATAGCTACCCCCTG |
| (Chothia) | | |
| BAP058-Clone N HC | | |
| SEQ ID NO: 81 (Kabat) | HCDR1 | AGCTACTGGATGTAC |
| SEQ ID NO: 82 (Kabat) | HCDR2 | AGAATCGACCCTAATAGCGGCTCTACTAAGTATAACGAG |
| | | AAGTTTAAGAAT |
| SEQ ID NO: 83 (Kabat) | HCDR3 | GACTATAGAAAGGGCCTGTACGCTATGGACTAC |
| SEQ ID NO: 84 | HCDR1 | GGCTACACCTTCACTAGCTAC |
| (Chothia) | | |
| SEQ ID NO: 85 | HCDR2 | GACCCTAATAGCGGCTCT |
| (Chothia) | | |
| SEQ ID NO: 83 | HCDR3 | GACTATAGAAAGGGCCTGTACGCTATGGACTAC |
| (Chothia) | | |
| BAP058-Clone N LC | | |
| SEQ ID NO: 86 (Kabat) | LCDR1 | AAAGCCTCTCAGGACGTGGGCACCGCCGTGGCC |
| SEQ ID NO: 87 (Kabat) | LCDR2 | TGGGCCTCTACTAGACACACC |
| SEQ ID NO: 88 (Kabat) | LCDR3 | CAGCAGTATAATAGCTACCCCCTGACC |
| SEQ ID NO: 89 | LCDR1 | TCTCAGGACGTGGGCACCGCC |
| (Chothia) | | |
| SEQ ID NO: 90 | LCDR2 | TGGGCCTCT |
| (Chothia) | | |
| SEQ ID NO: 91 | LCDR3 | TATAATAGCTACCCCCTG |
| (Chothia) | | |

Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 4.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 4.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 4.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 4.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082.

In one embodiment, the anti-PD-L1 antibody is an antibody that competes for binding with, and/or binds to the same epitope on PD-L1 as, one of the anti-PD-L1 antibodies described herein.

TABLE 4
Amino acid sequences of other exemplary anti-PD-Ll antibody molecules

| | | |
|---|---|---|
| Atezolizumab | | |
| SEQ ID NO: 92 | Heavy | EVQLVESGGGLVQPGGSLRLSCAASGFTFSDSWIHWVRQAPGKGLE |
| | chain | WVAWISPYGGSTYYADSVKGRFTISADTSKNTAYLQMNSLRAEDTA |
| | | VYYCARRHWPGGFDYWGQGTLVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYASTYRVVSVLTVLHQDWLNGKEYKCKVSN |
| | | KALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQG |
| | | NVFSCSVMHEALHNHYTQKSLSLSPGK |
| SEQ ID NO: 93 | Light | DIQMTQSPSSLSASVGDRVTITCRASQDVSTAVAWYQQKPGKAPKLLI |
| | chain | YSASFLYSGVPSRFSGSGSGTDFTLTISSLQPEDFATYYCQQYLYHPAT |
| | | FGQGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKV |
| | | YACEVTHQGLSSPVTKSFNRGEC |
| Avelumab | | |
| SEQ ID NO: 94 | Heavy | EVQLLESGGGLVQPGGSLRLSCAASGFTFSSYIMMWVRQAPGKGLE |
| | chain | WVSSIYPSGGITFYADTVKGRFTISRDNSKNTLYLQMNSLRAEDTAVY |
| | | YCARIKLGTVTTVDYWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGK |
| SEQ ID NO: 95 | Light | QSALTQPASVSGSPGQSITISCTGTSSDVGGYNYVSWYQQHPGKAPKL |
| | chain | MIYDVSNRPSGVSNRFSGSKSGNTASLTISGLQAEDEADYYCSSYTSS |
| | | STRVFGTGTKVTVLGQPKANPTVTLFPPSSEELQANKATLVCLISDFYP |
| | | GAVTVAWKADGSPVKAGVETTKPSKQSNNKYAASSYLSLTPEQWKS |
| | | HRSYSCQVTHEGSTVEKTVAPTECS |
| Durvalumab | | |
| SEQ ID NO: 96 | Heavy | EVQLVESGGGLVQPGGSLRLSCAASGFTFSRYWMSWVRQAPGKGLE |
| | chain | WVANIKQDGSEKYYVDSVKGRFTISRDNAKNSLYLQMNSLRAEDTA |
| | | VYYCAREGGWFGELAFDYWGQGTLVTVSSASTKGPSVFPLAPSSKST |
| | | SGGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPPCP |
| | | APEFEGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKV |
| | | SNKALPASIEKTISKAKGQPREPQVYTLPPSREEMTKNQVSLTCLVKG |
| | | FYPSDIAVEWESNGQPENNYKTTPPVLDSDGSFFLYSKLTVDKSRWQ |
| | | QGNVFSCSVMHEALHNHYTQKSLSLSPGK |
| SEQ ID NO: 97 | Light | EIVLTQSPGTLSLSPGERATLSCRASQRVSSSYLAWYQQKPGQAPRLLI |
| | chain | YDASSRATGIPDRFSGSGSGTDFTLTISRLEPEDFAVYYCQQYGSLPW |
| | | TFGQGTKVEIKRTVAAPSVFIFPPSDEQLKSGTASVVCLLNNFYPREA |
| | | KVQWKVDNALQSGNSQESVTEQDSKDSTYSLSSTLTLSKADYEKHK |
| | | VYACEVTHQGLSSPVTKSFNRGEC |
| BMS-936559 | | |
| SEQ ID NO: 98 | VH | QVQLVQSGAEVKKPGSSVKVSCKTSGDTFSTYAISWVRQAPGQGLE |
| | | WMGGIIPIFGKAHYAQKFQGRVTITADESTSTAYMELSSLRSEDTAVY |
| | | FCARKFHFVSGSPFGMDVWGQGTTVTVSS |
| SEQ ID NO: 99 | VL | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPTFG |
| | | QGTKVEIK |

### Antibodies and Antibody-Like Molecules

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full-length antibody which has an immunoglobulin Fc region). In an embodiment, an antibody molecule comprises a full-length antibody, or a full-length immunoglobulin chain. In an embodiment, an antibody molecule comprises an antigen binding or functional fragment of a full-length antibody, or a full-length immunoglobulin chain. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope (e.g., a first target) and a second immunoglobulin variable domain sequence of the plurality has binding specificity for a second epitope (e.g., a second target). In an embodiment, a multispecific antibody molecule is a bispecific antibody molecule.

In an embodiment, an antibody molecule is a monospecific antibody molecule and binds a single epitope (e.g., a single target such as TGFβ like NIS793). For example, a monospecific antibody molecule can have a plurality of immunoglobulin variable domain sequences, each of which binds the same epitope.

In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope (e.g., a first target) and a second immunoglobulin variable domain sequence of the plurality has binding specificity for a second epitope (e.g., a second target). In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule,

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment the first and second epitopes do not overlap. In an embodiment, the first and second epitopes are on different antigens, e.g., the different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the first epitope is located on TGFβ (1, 2, and/or 3) and the second epitope is located on PD-1 (or PD-L1 or PD-L2).

Protocols for generating multi-specific (e.g., bispecific or trispecific) or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US 2002/004587A1, US 2002/076406A1, US 2002/103345A1, US 2003/207346A1, US 2003/211078A1, US 2004/219643A1, US 2004/220388A1, US 2004/242847A1, US 2005/003403A1, US 2005/004352A1, US 2005/069552A1, US 2005/079170A1, US 2005/100543A1, US 2005/136049A1, US 2005/136051A1, US 2005/163782A1, US 2005/266425A1, US 2006/083747A1, US 2006/120960A1, US 2006/204493A1, US 2006/263367A1, US 2007/004909A1, US 2007/087381A1, US 2007/128150A1, US 2007/141049A1, US 2007/154901A1, US 2007/274985A1, US 2008/050370A1, US 2008/069820A1, US 2008/152645A1, US 2008/171855A1, US 2008/241884A1, US 2008/254512A1, US 2008/260738A1, US 2009/130106A1, US 2009/148905A1, US 2009/155275A1, US 2009/162359A1, US 2009/162360A1, US 2009/175851A1, US 2009/175867A1, US 2009/232811A1, US 2009/234105A1, US 2009/263392A1, US 2009/274649A1, EP 346087A2, WO 00/06605A2, WO 02/072635A2, WO 04/081051A1, WO 06/020258A2, WO 2007/044887A2, WO 2007/095338A2, WO 2007/137760A2, WO 2008/119353A1, WO 2009/021754A2, WO 2009/068630A1, WO 91/03493A1, WO 93/23537A1, WO 94/09131A1, WO 94/12625A2, WO 95/09917A1, WO 96/37621A2, WO 99/64460A1.

A “fusion protein” and a “fusion polypeptide” refer to a polypeptide having at least two portions covalently linked together, where each of the portions is a polypeptide having a different property. The property can be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which can be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region, e.g., IgG1, IgG2, IgG3, or IgG4. The antibody can also have a light chain, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies can be any as described in the art, or any future single domain antibodies. Single domain antibodies can be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any one of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the antibody molecules can include any combination of one or more Kabat CDRs and/or Chothia hypervariable loops. In one embodiment, the following definitions are used for the antibody molecules described in Table 1: HCDR1 according to the combined CDR definitions of both Kabat and Chothia, and HCCDRs 2-3 and LCCDRs 1-3 according the CDR definition of Kabat. Under all definitions, each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a target (such as TGFβ) or an epitope thereof. With respect to proteins (or protein mimetics), the antigen-binding site typically includes one or more loops (of at least four amino acids or amino acid mimics) that form an interface that binds to the target polypeptide. Typically, the antigen-binding site of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of another antibody molecule, e.g., an anti-TGFβ antibody molecule provided herein, to a target, e.g., TGFβ1, 2, or 3. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In some embodiments, a competition binding assay is a quantitative competition assay. In some embodiments, a first anti-TGFβ antibody molecule is said to compete for binding to the target with a second anti-TGFβ antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see e.g., Saleh et al., Cancer Immunol. Immunother. 32:180-190 (1990)) and also because of potential allergic reactions (see e.g., LoBuglio et al., Hybridoma, 5:5117-5123 (1986)).

The antibody molecule described can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02 809; Fuchs et al. (1991) Bio/Technology 9:1370-1 372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody can be replaced with at least a portion of a non-human CDR or only some of the CDRs can be replaced with non-human CDRs. It is only necessary to replace the number of CDRs required for binding of the humanized antibody to its target, e.g., TGFβ. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (see e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762.

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539. Winter describes a CDR-grafting method which can be used to prepare the humanized antibodies of the present invention (UK Patent Application GB 2188638A, filed on Mar. 26, 1987; Winter U.S. Pat. No. 5,225,539).

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089. Other techniques for humanizing antibodies are described in Padlan et al. EP 519596 A1, published on Dec. 23, 1992.

The antibody molecule can be a single chain antibody. A single-chain antibody (scFV) can be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region, e.g., the heavy chain constant regions of IgG1, IgG2, IgG3, IgG4, IgM, IgA1, IgA2, IgD, and IgE; particularly, e.g., the (human) heavy chain constant regions of IgG1, IgG2, IgG3, and IgG4. In another embodiment, the antibody molecule has a light chain constant region, e.g., the (human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment, the antibody has: effector function; and can fix complement. In other embodiments, the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260) Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention can be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody can be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule can be conjugated to another molecular entity, typically a label or a therapeutic (e.g., a cytotoxic or cytostatic) agent or moiety. Radioactive isotopes can be used in diagnostic or therapeutic applications.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody. As is discussed above, the antibody molecule can be conjugated to a therapeutic agent.

Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see, e.g., U.S. Pat. No. 5,208,020), CC-1065 (see, e.g., U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In one aspect, the disclosure provides a method of providing a target binding molecule that specifically binds to a target disclosed throughout. For example, the target binding molecule is an antibody molecule. The method includes: providing a target protein that comprises at least a portion of non-human protein, the portion being homologous to (at least 70, 75, 80, 81, 82, 83, 84, 85, 86, 87, 88, 89, 90, 91, 92, 93, 94, 95, 96, 97, 98, or 99% identical to) a corresponding portion of a human target protein, but differing by at least one amino acid (e.g., at least one, two, three, four, five, six, seven, eight, or nine amino acids); obtaining an antibody molecule that specifically binds to the antigen; and evaluating efficacy of the binding agent in modulating activity of the target protein. The method can further include administering the binding agent (e.g., antibody molecule) or a derivative (e.g., a humanized antibody molecule) to a human subject.

This disclosure provides an isolated nucleic acid molecule (i.e., a polynucleotide) encoding any of the antibody molecules described throughout. Also disclosed are vectors comprising the nucleic acid molecules and host cells thereof. The nucleic acid molecule includes but is not limited to RNA, genomic DNA and cDNA.

### Combinations

The methods of treatment described herein can comprise two or more other therapeutic agents, procedures or modalities administered in combination.

In some embodiments, the TGF-β inhibitor (e.g., NIS793) is administered in combination with a PD1 inhibitor (e.g., an anti-PD1 antibody molecule). In some embodiments, the TGF-β inhibitor is administered on the same day as the PD1 inhibitor. In some embodiments, the TGF-β inhibitor is administered after the administration of the PD1 inhibitor is started. In some embodiments, the TGF-β inhibitor is administered one hour after the administration of the anti-PD1 inhibitor is finished.

In some embodiments, the TGF-β inhibitor (e.g., NIS793) is administered at a dose between 1300 mg and 1500 mg (e.g., about 1400 mg), e.g., once every two weeks and the PD1 inhibitor (e.g., the anti-PD1 antibody molecule, e.g., spartalizumab) is administered at a dose between 300 mg to 500 mg (e.g., 400 mg), e.g., once every four weeks.

In some embodiments, the TGF-β inhibitor (e.g., NIS793) is administered at a dose between 1300 mg and 1500 mg (e.g., about 1400 mg), e.g., once every two weeks, and the PD1 inhibitor (e.g., the anti-PD1 antibody molecule, e.g., spartalizumab) is administered at a dose between 200 mg to 400 mg (e.g., 300 mg), e.g., once every three weeks.

In some embodiments, the TGF-β inhibitor (e.g., NIS793) is administered at a dose between 2000 mg and 2200 mg (e.g., about 2100 mg), e.g., once every two weeks, and the PD1 inhibitor (e.g., the anti-PD1 antibody molecule, e.g., spartalizumab) is administered at a dose between 300 mg to 500 mg (e.g., 400 mg), e.g., once every four weeks.

In some embodiments, the TGF-β inhibitor (e.g., NIS793) is administered at a dose between 2000 mg and 2200 mg (e.g., about 2100 mg), e.g., once every two weeks, and the PD1 inhibitor (e.g., the anti-PD1 antibody molecule, e.g., spartalizumab) is administered at a dose between 200 mg to 400 mg (e.g., 300 mg), e.g., once every three weeks.

In some embodiments, the TGF-β inhibitor (e.g., NIS793) is administered at a dose between 2000 mg and 2200 mg (e.g., about 2100 mg), e.g., once every three weeks, and the PD1 inhibitor (e.g., the anti-PD1 antibody molecule, e.g., spartalizumab) is administered at a dose between 300 mg to 500 mg (e.g., 400 mg), e.g., once every four weeks.

In some embodiments, the TGF-β inhibitor (e.g., NIS793) is administered at a dose between 2000 mg and 2200 mg (e.g., about 2100 mg), e.g., once every three weeks, and the PD1 inhibitor (e.g., the anti-PD1 antibody molecule, e.g., spartalizumab) is administered at a dose between 200 mg to 400 mg (e.g., 300 mg), e.g., once every three weeks.

In certain embodiments, the methods described herein can be administered with one or more of other therapeutic agents, including antibody molecules, chemotherapeutic agents, other anti-cancer therapies (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any one of the foregoing. The additional therapy can be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole-body irradiation (e.g., gamma irradiation). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

Alternatively, or in combination with the aforementioned, the methods described herein can be administered or used with, one or more of: an immunomodulator (e.g., an activator of a costimulatory molecule or an inhibitor of an inhibitory molecule, e.g., an immune checkpoint molecule); a vaccine, e.g., a therapeutic cancer vaccine; or other forms of cellular immunotherapy.

In certain embodiments, the combination described herein is administered or used in with a modulator of a costimulatory molecule or an inhibitory molecule, e.g., a co-inhibitory ligand or receptor.

In one embodiment, the combination described herein is administered or used in combination with an inhibitor of an inhibitory (or immune checkpoint) molecule PD-1, PD-L1, PD-L2, and/or TGFβ. In one embodiment, the inhibitor is an antibody or antibody fragment that binds to PD-1, PD-L1, PD-L2, or TGFβ.

For combination treatments, in some embodiments, the TGF-β inhibitor is administered on the same day as the checkpoint inhibitor. In other embodiments, the TGF-β inhibitor is administered before administration of the checkpoint inhibitor is completed. In additional embodiments, the TGF-β inhibitor is administered after administration of the checkpoint inhibitor is completed. In some embodiments, the TGF-β inhibitor is administered at the same time as the checkpoint inhibitor. In some embodiments, the TGF-β inhibitor is given until (partial or complete) remission. In some embodiments, the checkpoint inhibitor is given until (partial or complete) remission.

The compounds of the disclosure can be administered in therapeutically effective amounts in a combinational therapy with one or more therapeutic agents (pharmaceutical combinations) or modalities, e.g., non-drug therapies. For example, synergistic effects can occur with other cancer agents. Where the compounds of the application are administered in conjunction with other therapies, dosages of the co-administered compounds will of course vary depending on the type of co-drug employed, on the specific drug employed, on the condition being treated and so forth.

The compounds can be administered simultaneously (as a single preparation or separate preparation), sequentially, separately, or over a period of time to the other drug therapy or treatment modality. In general, a combination therapy envisions administration of two or more drugs during a single cycle or course of therapy. A therapeutic agent is, for example, a chemical compound, peptide, antibody, antibody fragment or nucleic acid, which is therapeutically active or enhances the therapeutic activity when administered to a patient in combination with a compound of the present disclosure.

In one aspect, the TGFβ inhibitors (and/or PD1, PD-L1, or PD-L2 inhibitor) can be combined with other therapeutic agents, such as other anti-cancer agents, anti-allergic agents, anti-nausea agents (or anti-emetics), pain relievers, cytoprotective agents, and combinations thereof.

In some embodiments, the TGFβ inhibitors are administered in combination with one or more second agent(s) selected from a PD-1 inhibitor, a PD-L1 inhibitor, a LAG-3 inhibitor, a cytokine, an A2A antagonist, a GITR agonist, a TIM-3 inhibitor, a STING agonist, and a TLR7 agonist, to treat a disease, e.g., cancer.

In another embodiment, one or more chemotherapeutic agents are used in combination with TGFβ inhibitors (and/or PD1, PD-L1, or PD-L2 inhibitor), for treating a disease, e.g., cancer, wherein said chemotherapeutic agents include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

In other embodiments, TGFβ inhibitors (and/or PD1, PD-L1, or PD-L2 inhibitor), of the present disclosure are used in combination with one or more other anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In other embodiments, TGFβ inhibitors (and/or PD1, PD-L1, or PD-L2 inhibitor), of the present disclosure are used in combination with one or more tyrosine kinase inhibitors, including but not limited to, EGFR inhibitors, Her3 inhibitors, IGFR inhibitors, and Met inhibitors, for treating a disease, e.g., cancer.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3 S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)-N-(3-Chlorophenyl)-3-({3,5-dimethyl-[4(4-methylpiperazin-1-yl)carbonyl]-1H-pyrrol-2-yl}methylene)-N-methyl-2-oxoindoline-5-sulfonamide (SU11274); (3Z)-N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1Hpyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl) pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another embodiment, the TGFβ inhibitors (and/or PD1, PD-L1, or PD-L2 inhibitor) are used in combination with one or more proliferation signalling pathway inhibitors, including but not limited to, MEK inhibitors, BRAF inhibitors, PI3K/Akt inhibitors, SHP2 inhibitors, and also mTOR inhibitors, and CDK inhibitors, for treating a disease, e.g., cancer.

For example, mitogen-activated protein kinase (MEK) inhibitors include but are not limited to, XL-518 (also known as GDC-0973, CAS No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)-N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-□-aspartylL-serine-, inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, TGFβ inhibitors (and/or PD1, PD-L1, or PD-L2 inhibitor), of the present disclosure are used in combination with one or more pro-apoptotics, including but not limited to, IAP inhibitors, BCL2 inhibitors, MCL1 inhibitors, TRAIL agents, CHK inhibitors, for treating a disease, e.g., cancer.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N-[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 318)), and CBP501 ((d-Bpa)sws(d-Phe-F5)(d-Cha)rrrqrr).

In a further embodiment, TGFβ inhibitors (and/or PD1, PD-L1, or PD-L2 inhibitor) of the present disclosure are used in combination with one or more immunomodulators (e.g., one or more of an activator of a costimulatory molecule or an inhibitor of an immune checkpoint molecule), for treating a disease, e.g., cancer.

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Agonists

In some embodiments, a GITR agonist is used in combination with a TGFβ inhibitors (and/or PD1, PD-L1, or PD-L2 inhibitor), for treating a disease, e.g., cancer. In some embodiments, the GITR agonist is GWN323 (Novartis), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy”.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of MAB7 disclosed in Table 5), or encoded by a nucleotide sequence shown in Table 5. In some embodiments, the CDRs are according to the Kabat definition. In some embodiments, the CDRs are according to the Chothia definition. In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence or encoded by a nucleotide sequence.

In one embodiment, the anti-GITR antibody molecule comprises a heavy chain variable region (VH) comprising a VHCDR1 amino acid sequence of SEQ ID NO: 109, a VHCDR2 amino acid sequence of SEQ ID NO: 111, and a VHCDR3 amino acid sequence of SEQ ID NO: 113; and a light chain variable region (VL) comprising a VLCDR1 amino acid sequence of SEQ ID NO: 114, a VLCDR2 amino acid sequence of SEQ ID NO: 116, and a VLCDR3 amino acid sequence of SEQ ID NO: 118, each disclosed in Table 5.

In one embodiment, the anti-GITR antibody molecule comprises a VH comprising the amino acid sequence of SEQ ID NO: 101, or an amino acid sequence at least 85%, 90%, 95%, or 99% identical or higher to SEQ ID NO: 101. In one embodiment, the anti-GITR antibody molecule comprises a VL comprising the amino acid sequence of SEQ ID NO: 102, or an amino acid sequence at least 85%, 90%, 95%, or 99% identical or higher to SEQ ID NO: 102. In one embodiment, the anti-GITR antibody molecule comprises a VH comprising the amino acid sequence of SEQ ID NO: 101 and a VL comprising the amino acid sequence of SEQ ID NO: 102.

In one embodiment, the antibody molecule comprises a VH encoded by the nucleotide sequence of SEQ ID NO: 105, or a nucleotide sequence at least 85%, 90%, 95%, or 99% identical or higher to SEQ ID NO: 105. In one embodiment, the antibody molecule comprises a VL encoded by the nucleotide sequence of SEQ ID NO: 106, or a nucleotide sequence at least 85%, 90%, 95%, or 99% identical or higher to SEQ ID NO: 106. In one embodiment, the antibody molecule comprises a VH encoded by the nucleotide sequence of SEQ ID NO: 105 and a VL encoded by the nucleotide sequence of SEQ ID NO: 106.

In one embodiment, the anti-GITR antibody molecule comprises a heavy chain comprising the amino acid sequence of SEQ ID NO: 103, or an amino acid sequence at least 85%, 90%, 95%, or 99% identical or higher to SEQ ID NO: 103. In one embodiment, the anti-GITR antibody molecule comprises a light chain comprising the amino acid sequence of SEQ ID NO: 104, or an amino acid sequence at least 85%, 90%, 95%, or 99% identical or higher to SEQ ID NO: 104. In one embodiment, the anti-GITR antibody molecule comprises a heavy chain comprising the amino acid sequence of SEQ ID NO: 103 and a light chain comprising the amino acid sequence of SEQ ID NO: 104.

In one embodiment, the antibody molecule comprises a heavy chain encoded by the nucleotide sequence of SEQ ID NO: 107, or a nucleotide sequence at least 85%, 90%, 95%, or 99% identical or higher to SEQ ID NO: 107. In one embodiment, the antibody molecule comprises a light chain encoded by the nucleotide sequence of SEQ ID NO: 108, or a nucleotide sequence at least 85%, 90%, 95%, or 99% identical or higher to SEQ ID NO: 108. In one embodiment, the antibody molecule comprises a heavy chain encoded by the nucleotide sequence of SEQ ID NO: 107 and a light chain encoded by the nucleotide sequence of SEQ ID NO: 108.

The antibody molecules described herein can be made by vectors, host cells, and methods described in WO 2016/057846.

TABLE 5
Amino acid and nucleotide sequences of exemplary anti-GITR antibody molecule

| | | |
|---|---|---|
| MAB7 | | |
| SEQ ID NO: 101 | VH | EVQLVESGGGLVQSGGSLRLSCAASGFSLSSYGVDWVRQAPGKGLEWV |
| | | GVIWGGGGTYYASSLMGRFTISRDNSKNTLYLQMNSLRAEDTAVYYCA |
| | | RHAYGHDGGFAMDYWGQGTLVTVSS |
| SEQ ID NO: 102 | VL | EIVMTQSPATLSVSPGERATLSCRASESVSSNVAWYQQRPGQAPRLLIYG |
| | | ASNRATGIPARFSGSGSGTDFTLTISRLEPEDFAVYYCGQSYSYPFTFGQG |
| | | TKLEIK |
| SEQ ID NO: 103 | Heavy | EVQLVESGGGLVQSGGSLRLSCAASGFSLSSYGVDWVRQAPGKGLEWV |
| | Chain | GVIWGGGGTYYASSLMGRFTISRDNSKNTLYLQMNSLRAEDTAVYYCA |
| | | RHAYGHDGGFAMDYWGQGTLVTVSSASTKGPSVFPLAPSSKSTSGGTA |
| | | ALGCLVKDYFPEPVTVSWNSGALTSGVHTFPAVLQSSGLYSLSSVVTVPS |
| | | SSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNH |
| | | YTQKSLSLSPGK |
| SEQ ID NO: 104 | Light | EIVMTQSPATLSVSPGERATLSCRASESVSSNVAWYQQRPGQAPRLLIYG |
| | Chain | ASNRATGIPARFSGSGSGTDFTLTISRLEPEDFAVYYCGQSYSYPFTFGQG |
| | | TKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| SEQ ID NO: 105 | DNA VH | GAGGTGCAGCTGGTGGAATCTGGCGGCGGACTGGTGCAGTCCGGCGG |
| | | CTCTCTGAGACTGTCTTGCGCTGCCTCCGGCTTCTCCCTGTCCTCTTAC |
| | | GGCGTGGACTGGGTGCGACAGGCCCCTGGCAAGGGCCTGGAATGGGT |
| | | GGGAGTGATCTGGGGCGGAGGCGGCACCTACTACGCCTCTTCCCTGA |
| | | TGGGCCGGTTCACCATCTCCCGGGACAACTCCAAGAACACCCTGTAC |
| | | CTGCAGATGAACTCCCTGCGGGCCGAGGACACCGCCGTGTACTACTG |
| | | CGCCAGACACGCCTACGGCCACGACGGCGGCTTCGCCATGGATTATT |
| | | GGGGCCAGGGCACCCTGGTGACAGTGTCCTCC |
| SEQ ID NO: 106 | DNA VL | GAGATCGTGATGACCCAGTCCCCCGCCACCCTGTCTGTGTCTCCCGGC |
| | | GAGAGAGCCACCCTGAGCTGCAGAGCCTCCGAGTCCGTGTCCTCCAA |
| | | CGTGGCCTGGTATCAGCAGAGACCTGGTCAGGCCCCTCGGCTGCTGA |
| | | TCTACGGCGCCTCTAACCGGGCCACCGGCATCCCTGCCAGATTCTCCG |
| | | GCTCCGGCAGCGGCACCGACTTCACCCTGACCATCTCCCGGCTGGAA |
| | | CCCGAGGACTTCGCCGTGTACTACTGCGGCCAGTCCTACTCATACCCC |
| | | TTCACCTTCGGCCAGGGCACCAAGCTGGAAATCAAG |
| SEQ ID NO: 107 | DNA | GAGGTGCAGCTGGTGGAATCTGGCGGCGGACTGGTGCAGTCCGGCGG |
| | Heavy | CTCTCTGAGACTGTCTTGCGCTGCCTCCGGCTTCTCCCTGTCCTCTTAC |
| | Chain | GGCGTGGACTGGGTGCGACAGGCCCCTGGCAAGGGCCTGGAATGGGT |
| | | GGGAGTGATCTGGGGCGGAGGCGGCACCTACTACGCCTCTTCCCTGA |
| | | TGGGCCGGTTCACCATCTCCCGGGACAACTCCAAGAACACCCTGTAC |
| | | CTGCAGATGAACTCCCTGCGGGCCGAGGACACCGCCGTGTACTACTG |
| | | CGCCAGACACGCCTACGGCCACGACGGCGGCTTCGCCATGGATTATT |
| | | GGGGCCAGGGCACCCTGGTGACAGTGTCCTCCGCTAGCACCAAGGGC |
| | | CCAAGTGTGTTTCCCCTGGCCCCCAGCAGCAAGTCTACTTCCGGCGGA |
| | | ACTGCTGCCCTGGGTTGCCTGGTGAAGGACTACTTCCCCGAGCCCGTG |
| | | ACAGTGTCCTGGAACTCTGGGGCTCTGACTTCCGGCGTGCACACCTTC |
| | | CCCGCCGTGCTGCAGAGCAGCGGCCTGTACAGCCTGAGCAGCGTGGT |
| | | GACAGTGCCCTCCAGCTCTCTGGGAACCCAGACCTATATCTGCAACGT |
| | | GAACCACAAGCCCAGCAACACCAAGGTGGACAAGAGAGTGGAGCCC |
| | | AAGAGCTGCGACAAGACCCACACCTGCCCCCCCTGCCCAGCTCCAGA |
| | | ACTGCTGGGAGGGCCTTCCGTGTTCCTGTTCCCCCCCAAGCCCAAGGA |
| | | CACCCTGATGATCAGCAGGACCCCCGAGGTGACCTGCGTGGTGGTGG |
| | | ACGTGTCCCACGAGGACCCAGAGGTGAAGTTCAACTGGTACGTGGAC |
| | | GGCGTGGAGGTGCACAACGCCAAGACCAAGCCCAGAGAGGAGCAGT |
| | | ACAACAGCACCTACAGGGTGGTGTCCGTGCTGACCGTGCTGCACCAG |
| | | GACTGGCTGAACGGCAAAGAATACAAGTGCAAAGTCTCCAACAAGG |
| | | CCCTGCCAGCCCCAATCGAAAAGACAATCAGCAAGGCCAAGGGCCA |
| | | GCCACGGGAGCCCCAGGTGTACACCCTGCCCCCCAGCCGGGAGGAGA |
| | | TGACCAAGAACCAGGTGTCCCTGACCTGTCTGGTGAAGGGCTTCTAC |
| | | CCCAGCGATATCGCCGTGGAGTGGGAGAGCAACGGCCAGCCCGAGA |
| | | ACAACTACAAGACCACCCCCCCAGTGCTGGACAGCGACGGCAGCTTC |
| | | TTCCTGTACAGCAAGCTGACCGTGGACAAGTCCAGGTGGCAGCAGGG |
| | | CAACGTGTTCAGCTGCAGCGTGATGCACGAGGCCCTGCACAACCACT |
| | | ACACCCAGAAGTCCCTGAGCCTGAGCCCCGGCAAG |
| SEQ ID NO: 108 | DNA | GAGATCGTGATGACCCAGTCCCCCGCCACCCTGTCTGTGTCTCCCGGC |
| | Light | GAGAGAGCCACCCTGAGCTGCAGAGCCTCCGAGTCCGTGTCCTCCAA |
| | Chain | CGTGGCCTGGTATCAGCAGAGACCTGGTCAGGCCCCTCGGCTGCTGA |
| | | TCTACGGCGCCTCTAACCGGGCCACCGGCATCCCTGCCAGATTCTCCG |
| | | GCTCCGGCAGCGGCACCGACTTCACCCTGACCATCTCCCGGCTGGAA |
| | | CCCGAGGACTTCGCCGTGTACTACTGCGGCCAGTCCTACTCATACCCC |
| | | TTCACCTTCGGCCAGGGCACCAAGCTGGAAATCAAGCGTACGGTGGC |
| | | CGCTCCCAGCGTGTTCATCTTCCCCCCCAGCGACGAGCAGCTGAAGA |
| | | GCGGCACCGCCAGCGTGGTGTGCCTGCTGAACAACTTCTACCCCCGG |
| | | GAGGCCAAGGTGCAGTGGAAGGTGGACAACGCCCTGCAGAGCGGCA |
| | | ACAGCCAGGAGAGCGTCACCGAGCAGGACAGCAAGGACTCCACCTA |
| | | CAGCCTGAGCAGCACCCTGACCCTGAGCAAGGCCGACTACGAGAAGC |
| | | ATAAGGTGTACGCCTGCGAGGTGACCCACCAGGGCCTGTCCAGCCCC |
| | | GTGACCAAGAGCTTCAACAGGGGCGAGTGC |
| SEQ ID NO: 109 | HCDR1 | SYGVD |
| (KABAT) | | |
| SEQ ID NO: 110 | HCDR1 | GFSLSSY |
| (CHOTHIA) | | |
| SEQ ID NO: 111 | HCDR2 | VIWGGGGTYYASSLMG |
| (KABAT) | | |
| SEQ ID NO: 112 | HCDR2 | WGGGG |
| (CHOTHIA) | | |
| SEQ ID NO: 113 | HCDR3 | HAYGHDGGFAMDY |
| (KABAT) | | |
| SEQ ID NO: 113 | HCDR3 | HAYGHDGGFAMDY |
| (CHOTHIA) | | |
| SEQ ID NO: 114 | LCDR1 | RASESVSSNVA |
| (KABAT) | | |
| SEQ ID NO: 115 | LCDR1 | SESVSSN |
| (CHOTHIA) | | |
| SEQ ID NO: 116 | LCDR2 | GASNRAT |
| (KABAT) | | |
| SEQ ID NO: 117 | LCDR2 | GAS |
| (CHOTHIA) | | |
| SEQ ID NO: 118 | LCDR3 | GQSYSYPFT |
| (KABAT) | | |
| SEQ ID NO: 119 | LCDR3 | SYSYPF |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 6.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signalling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 6
Amino acid sequence of other exemplary
anti-GITR antibody molecules

| BMS-986156 | | |
|---|---|---|
| SEQ ID NO: 120 | VH | QVQLVESGGGVVQPGRSLRLSCAASGFTFSSYG |
| | | MHWVRQAPGKGLEWVAVIWYEGSNKYYADSVKG |
| | | RFTISRDNSKNTLYLQMNSLRAEDTAVYYCARG |
| | | GSMVRGDYYYGMDVWGQGTTVTVSS |
| SEQ ID NO: 121 | VL | AIQLTQSPSSLSASVGDRVTITCRASQGISSAL |
| | | AWYQQKPGKAPKLLIYDASSLESGVPSRFSGSG |
| | | SGTDFTLTISSLQPEDFATYYCQQFNSYPYTFG |
| | | QGTKLEIK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)2, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TGFβ, TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and TGFβ. In another embodiment, the bispecific antibody molecule binds to PD-1 and TGFβ. Any combination of the aforesaid molecules can be made in a multispecific antibody molecule, e.g., a trispecific antibody that includes a first binding specificity to PD-1 or PD-1, and a second and third binding specificities to two or more of TGFβ, TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of TGFβ, LAG-3, TIM-3 or CTLA4. In an exemplary embodiment, the inhibitor of PD-1 or PD-L1, e.g., the anti-TGFβ or anti-PD-1 or PD-L1 antibody molecule, is administered in combination with a LAG-3 inhibitor, e.g., an anti-LAG-3 antibody molecule. In another embodiment, the inhibitor of TGFβ, PD-1 or PD-L1, e.g., the anti-TGFβ or anti-PD-1 or PD-L1 antibody molecule, is administered in combination with a TIM-3 inhibitor, e.g., an anti-TIM-3 antibody molecule. In yet other embodiments, the inhibitor of TGFβ, PD-1 or PD-L1, e.g., the anti-TGFβ or anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present disclosure. Any of the antibody molecules known in the art or disclosed herein can be used in the aforesaid combinations of inhibitors of checkpoint molecule.

### CTLA-4 Inhibitors

In some embodiments, TGFβ inhibitors (and/or PD1, PD-L1, or PD-L2 inhibitor), of the present disclosure are used in combination with a CTLA-4 inhibitor to treat a disease, e.g., cancer. In some embodiments, the PD-1 inhibitor is selected from Ipilimumab (MDX-010, MDX-101, or Yervoy, Bristol-Myers Squibb), tremelilumab (ticilimumab Pfizer/AstraZeneca), AGEN1181 (Agenus), Zalifrelimab (AGEN1884, Agenus), IBI310 (Innovent Biologics),

### LAG-3 Inhibitors

In some embodiments, TGFβ inhibitors (and/or PD1, PD-L1, or PD-L2 inhibitor), of the present disclosure are used in combination with a LAG-3 inhibitor to treat a disease, e.g., cancer. In some embodiments, the LAG-3 inhibitor is selected from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof”.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP050-Clone I or BAP050-Clone J disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GFTLTNYGMN (SEQ ID NO: 122). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-LAG-3 antibody molecule comprises a heavy chain variable region (VH) comprising a VHCDR1 amino acid sequence of SEQ ID NO: 123, a VHCDR2 amino acid sequence of SEQ ID NO: 124, and a VHCDR3 amino acid sequence of SEQ ID NO: 125; and a light chain variable region (VL) comprising a VLCDR1 amino acid sequence of SEQ ID NO: 132, a VLCDR2 amino acid sequence of SEQ ID NO: 133, and a VLCDR3 amino acid sequence of SEQ ID NO: 134, each disclosed in Table 7.

In one embodiment, the anti-LAG-3 antibody molecule comprises a VH comprising a VHCDR1 encoded by the nucleotide sequence of SEQ ID NO: 158 or 159, a VHCDR2 encoded by the nucleotide sequence of SEQ ID NO: 160 or 161, and a VHCDR3 encoded by the nucleotide sequence of SEQ ID NO: 162 or 163; and a VL comprising a VLCDR1 encoded by the nucleotide sequence of SEQ ID NO: 168 or 169, a VLCDR2 encoded by the nucleotide sequence of SEQ ID NO: 170 or 171, and a VLCDR3 encoded by the nucleotide sequence of SEQ ID NO: 172 or 173, each disclosed in Table 7. In one embodiment, the anti-LAG-3 antibody molecule comprises a VH comprising a VHCDR1 encoded by the nucleotide sequence of SEQ ID NO: 180 or 159, a VHCDR2 encoded by the nucleotide sequence of SEQ ID NO: 181 or 161, and a VHCDR3 encoded by the nucleotide sequence of SEQ ID NO: 182 or 163; and a VL comprising a VLCDR1 encoded by the nucleotide sequence of SEQ ID NO: 168 or 169, a VLCDR2 encoded by the nucleotide sequence of SEQ ID NO: 170 or 171, and a VLCDR3 encoded by the nucleotide sequence of SEQ ID NO: 172 or 173, each disclosed in Table 7.

In one embodiment, the anti-LAG-3 antibody molecule comprises a VH comprising the amino acid sequence of SEQ ID NO: 128, or an amino acid sequence at least 85%, 90%, 95%, or 99% identical or higher to SEQ ID NO: 128. In one embodiment, the anti-LAG-3 antibody molecule comprises a VL comprising the amino acid sequence of SEQ ID NO: 140, or an amino acid sequence at least 85%, 90%, 95%, or 99% identical or higher to SEQ ID NO: 140. In one embodiment, the anti-LAG-3 antibody molecule comprises a VH comprising the amino acid sequence of SEQ ID NO: 146, or an amino acid sequence at least 85%, 90%, 95%, or 99% identical or higher to SEQ ID NO: 146. In one embodiment, the anti-LAG-3 antibody molecule comprises a VL comprising the amino acid sequence of SEQ ID NO: 152, or an amino acid sequence at least 85%, 90%, 95%, or 99% identical or higher to SEQ ID NO: 152. In one embodiment, the anti-LAG-3 antibody molecule comprises a VH comprising the amino acid sequence of SEQ ID NO: 128 and a VL comprising the amino acid sequence of SEQ ID NO: 140. In one embodiment, the anti-LAG-3 antibody molecule comprises a VH comprising the amino acid sequence of SEQ ID NO: 146 and a VL comprising the amino acid sequence of SEQ ID NO: 152.

In one embodiment, the antibody molecule comprises a VH encoded by the nucleotide sequence of SEQ ID NO: 129 or 130, or a nucleotide sequence at least 85%, 90%, 95%, or 99% identical or higher to SEQ ID NO: 129 or 130. In one embodiment, the antibody molecule comprises a VL encoded by the nucleotide sequence of SEQ ID NO: 141 or 142, or a nucleotide sequence at least 85%, 90%, 95%, or 99% identical or higher to SEQ ID NO: 141 or 142. In one embodiment, the antibody molecule comprises a VH encoded by the nucleotide sequence of SEQ ID NO: 147 or 148, or a nucleotide sequence at least 85%, 90%, 95%, or 99% identical or higher to SEQ ID NO: 147 or 148. In one embodiment, the antibody molecule comprises a VL encoded by the nucleotide sequence of SEQ ID NO: 153 or 154, or a nucleotide sequence at least 85%, 90%, 95%, or 99% identical or higher to SEQ ID NO: 153 or 154. In one embodiment, the antibody molecule comprises a VH encoded by the nucleotide sequence of SEQ ID NO: 129 or 130 and a VL encoded by the nucleotide sequence of SEQ ID NO: 141 or 142. In one embodiment, the antibody molecule comprises a VH encoded by the nucleotide sequence of SEQ ID NO: 147 or 148 and a VL encoded by the nucleotide sequence of SEQ ID NO: 153 or 154.

In one embodiment, the anti-LAG-3 antibody molecule comprises a heavy chain comprising the amino acid sequence of SEQ ID NO: 131, or an amino acid sequence at least 85%, 90%, 95%, or 99% identical or higher to SEQ ID NO: 131. In one embodiment, the anti-LAG-3 antibody molecule comprises a light chain comprising the amino acid sequence of SEQ ID NO: 143, or an amino acid sequence at least 85%, 90%, 95%, or 99% identical or higher to SEQ ID NO: 143. In one embodiment, the anti-LAG-3 antibody molecule comprises a heavy chain comprising the amino acid sequence of SEQ ID NO: 149, or an amino acid sequence at least 85%, 90%, 95%, or 99% identical or higher to SEQ ID NO: 149. In one embodiment, the anti-LAG-3 antibody molecule comprises a light chain comprising the amino acid sequence of SEQ ID NO: 155, or an amino acid sequence at least 85%, 90%, 95%, or 99% identical or higher to SEQ ID NO: 155. In one embodiment, the anti-LAG-3 antibody molecule comprises a heavy chain comprising the amino acid sequence of SEQ ID NO: 131 and a light chain comprising the amino acid sequence of SEQ ID NO: 143. In one embodiment, the anti-LAG-3 antibody molecule comprises a heavy chain comprising the amino acid sequence of SEQ ID NO: 149 and a light chain comprising the amino acid sequence of SEQ ID NO: 155.

In one embodiment, the antibody molecule comprises a heavy chain encoded by the nucleotide sequence of SEQ ID NO: 138 or 139, or a nucleotide sequence at least 85%, 90%, 95%, or 99% identical or higher to SEQ ID NO: 138 or 139. In one embodiment, the antibody molecule comprises a light chain encoded by the nucleotide sequence of SEQ ID NO: 144 or 145, or a nucleotide sequence at least 85%, 90%, 95%, or 99% identical or higher to SEQ ID NO: 144 or 145. In one embodiment, the antibody molecule comprises a heavy chain encoded by the nucleotide sequence of SEQ ID NO: 150 or 151, or a nucleotide sequence at least 85%, 90%, 95%, or 99% identical or higher to SEQ ID NO: 150 or 151. In one embodiment, the antibody molecule comprises a light chain encoded by the nucleotide sequence of SEQ ID NO: 156 or 157, or a nucleotide sequence at least 85%, 90%, 95%, or 99% identical or higher to SEQ ID NO: 156 or 157. In one embodiment, the antibody molecule comprises a heavy chain encoded by the nucleotide sequence of SEQ ID NO: 138 or 139 and a light chain encoded by the nucleotide sequence of SEQ ID NO: 144 or 145. In one embodiment, the antibody molecule comprises a heavy chain encoded by the nucleotide sequence of SEQ ID NO: 150 or 151 and a light chain encoded by the nucleotide sequence of SEQ ID NO: 156 or 157.

The antibody molecules described herein can be made by vectors, host cells, and methods described in US 2015/0259420.

TABLE 7
Amino acid and nucleotide sequences of
exemplary anti-LAG-3 antibody molecules

| | | |
|---|---|---|
| BAP050-Clone I HC | | |
| SEQ ID NO: 123 | HCDR1 | NYGMN |
| (Kabat) | | |
| SEQ ID NO: 124 | HCDR2 | WINTDTGEPTYADDFKG |
| (Kabat) | | |
| SEQ ID NO: 125 | HCDR3 | NPPYYYGTNNAEAMDY |
| (Kabat) | | |
| SEQ ID NO: 126 | HCDR1 | GFTLTNY |
| (Chothia) | | |
| SEQ ID NO: 127 | HCDR2 | NTDTGE |
| (Chothia) | | |
| SEQ ID NO: 125 | HCDR3 | NPPYYYGTNNAEAMDY |
| (Chothia) | | |
| SEQ ID NO: 128 | VH | QVQLVQSGAEVKKPGASVKVSCKASGFTLTNYGMNWVRQAR |
| | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| SEQ ID NO: 129 | DNA | CAAGTGCAGCTGGTGCAGTCGGGAGCCGAAGTGAAGAAGCC |
| | VH | TGGAGCCTCGGTGAAGGTGTCGTGCAAGGCATCCGGATTCA |
| | | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGC |
| SEQ ID NO: 130 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 131 | Heavy | TQVQLVQSGAEVKKPGASVKVSCKASGFTLTNYGMNWVRQAR |
| | chain | GQRLEWIGWINTDTGEPTYADDFKGRFVFSLDTSVSTAYLQISS |
| | | LKAEDTAVYYCARNPPYYYGTNNAEAMDYWGQGTTVTVSSA |
| | | STKGPSVFPLAPCSRSTSESTAALGCLVKDYFPEPVTVSWNSGA |
| | | LTSGVHTFPAVLQSSGLYSLSSVVTVPSSSLGTKTYTCNVDHKP |
| | | SNTKVDKRVESKYGPPCPPCPAPEFLGGPSVFLFPPKPKDTLMIS |
| | | RTPEVTCVVVDVSQEDPEVQFNWYVDGVEVHNAKTKPREEQF |
| | | NSTYRVVSVLTVLHQDWLNGKEYKCKVSNKGLPSSIEKTISKA |
| | | KGQPREPQVYTLPPSQEEMTKNQVSLTCLVKGFYPSDIAVEWE |
| | | SNGQPENNYKTTPPVLDSDGSFFLYSRLTVDKSRWQEGNVFSC |
| | | SVMHEALHNHYTQKSLSLSLG |
| SEQ ID NO: 138 | DNA | CAAGTGCAGCTGGTGCAGTCGGGAGCCGAAGTGAAGAAGCC |
| | heavy | TGGAGCCTCGGTGAAGGTGTCGTGCAAGGCATCCGGATTCA |
| | chain | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGCGCGTCCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 139 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCAC |
| | chain | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone I LC | | |
| SEQ ID NO: 132 | LCDR1 | SSSQDSINYLN |
| (Kabat) | | |
| SEQ ID NO: 133 | LCDR2 | YTSTLHL |
| (Kabat) | | |
| SEQ ID NO: 134 | LCDR3 | QQYYNLPWT |
| (Kabat) | | |
| SEQ ID NO: 135 | LCDR1 | SQDISNY |
| (Chothia) | | |
| SEQ ID NO: 136 | LCDR2 | YTS |
| (Chothia) | | |
| SEQ ID NO: 137 | LCDR3 | YYNLPW |
| (Chothia) | | |
| SEQ ID NO: 140 | VL | DIQMTQSPSSLSASVGDRVTITCSSSQDISNYLNWYLQKPGQSP |
| | | QLLIYYTSTLHLGVPSRFSGSGSGTEFTLTISSLQPDDFATYYCQ |
| | | QYYNLPWTFGQGTKVEIK |
| SEQ ID NO: 141 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAG |
| SEQ ID NO: 142 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAG |
| SEQ ID NO: 143 | Light | DIQMTQSPSSLSASVGDRVTITCSSSQDISNYLNWYLQKPGQSP |
| | chain | QLLIYYTSTLHLGVPSRFSGSGSGTEFTLTISSLQPDDFATYYCQ |
| | DNA | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 144 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAGCGTACGGTGGCC |
| | | GCTCCCAGCGTGTTCATCTTCCCCCCCAGCGACGAGCAGCTG |
| | | AAGAGCGGCACCGCCAGCGTGGTGTGCCTGCTGAACAACTT |
| | | CTACCCCCGGGAGGCCAAGGTGCAGTGGAAGGTGGACAACG |
| | | CCCTGCAGAGCGGCAACAGCCAGGAGAGCGTCACCGAGCA |
| | | GGACAGCAAGGACTCCACCTACAGCCTGAGCAGCACCCTGA |
| | | CCCTGAGCAAGGCCGACTACGAGAAGCATAAGGTGTACGCC |
| | | TGCGAGGTGACCCACCAGGGCCTGTCCAGCCCCGTGACCAA |
| | | GAGCTTCAACAGGGGCGAGTGC |
| SEQ ID NO: 145 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAGCGTACGGTGGCC |
| | | GCTCCCAGCGTGTTCATCTTCCCCCCAAGCGACGAGCAGCTG |
| | | AAGAGCGGCACCGCCAGCGTGGTGTGTCTGCTGAACAACTT |
| | | CTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone J HC | | |
| SEQ ID NO: 123 | HCDR1 | NYGMN |
| (Kabat) | | |
| SEQ ID NO: 124 | HCDR2 | WINTDTGEPTYADDFKG |
| (Kabat) | | |
| SEQ ID NO: 125 | HCDR3 | NPPYYYGTNNAEAMDY |
| (Kabat) | | |
| SEQ ID NO: 126 | HCDR1 | GFTLTNY |
| (Chothia) | | |
| SEQ ID NO: 127 | HCDR2 | NTDTGE |
| (Chothia) | | |
| SEQ ID NO: 125 | HCDR3 | NPPYYYGTNNAEAMDY |
| (Chothia) | | |
| SEQ ID NO: 146 | VH | QVQLVQSGAEVKKPGASVKVSCKASGFTLTNYGMNWVRQAP |
| | | GQGLEWMGWINTDTGEPTYADDFKGRFVFSLDTSVSTAYLQIS |
| | | SLKAEDTAVYYCARNPPYYYGTNNAEAMDYWGQGTTVTVSS |
| SEQ ID NO: 147 | DNA | CAGGTGCAGCTGGTGCAGTCAGGCGCCGAAGTGAAGAAACC |
| | VH | CGGCGCTAGTGTGAAAGTCAGCTGTAAAGCTAGTGGCTTCA |
| | | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGC |
| SEQ ID NO: 148 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 149 | Heavy | QVQLVQSGAEVKKPGASVKVSCKASGFTLTNYGMNWVRQAP |
| | chain | GQGLEWMGWINTDTGEPTYADDFKGRFVFSLDTSVSTAYLQIS |
| | DNA | SLKAEDTAVYYCARNPPYYYGTNNAEAMDYWGQGTTVTVSS |
| | | ASTKGPSVFPLAPCSRSTSESTAALGCLVKDYFPEPVTVSWNSG |
| | | ALTSGVHTFPAVLQSSGLYSLSSVVTVPSSSLGTKTYTCNVDHK |
| | | PSNTKVDKRVESKYGPPCPPCPAPEFLGGPSVFLFPPKPKDTLMI |
| | | SRTPEVTCVVVDVSQEDPEVQFNWYVDGVEVHNAKTKPREEQ |
| | | AKGQPREPQVYTLPPSQEEMTKNQVSLTCLVKGFYPSDIAVEW |
| | | ESNGQPENNYKTTPPVLDSDGSFFLYSRLTVDKSRWQEGNVFS |
| | | CSVMHEALHNHYTQKSLSLSLG |
| SEQ ID NO: 150 | DNA | CAGGTGCAGCTGGTGCAGTCAGGCGCCGAAGTGAAGAAACC |
| | heavy | CGGCGCTAGTGTGAAAGTCAGCTGTAAAGCTAGTGGCTTCA |
| | chain | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGCGCTAGCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 151 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCAC |
| | chain | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone J LC | | |
| SEQ ID NO: 132 | LCDR1 | SSSQDISNYLN |
| (Kabat) | | |
| SEQ ID NO: 133 | LCDR2 | YTSTLHL |
| (Kabat) | | |
| SEQ ID NO: 134 | LCDR3 | QQYYNLPWT |
| (Kabat) | | |
| SEQ ID NO: 135 | LCDR1 | SQDISNY |
| (Chothia) | | |
| SEQ ID NO: 136 | LCDR2 | YTS |
| (Chothia) | | |
| SEQ ID NO: 137 | LCDR3 | YYNLPW |
| (Chothia) | | |
| SEQ ID NO: 152 | VL | DIQMTQSPSSLSASVGDRVTITCSSSQDISNYLNWYQQKPGKAP |
| | | KLLIYYTSTLHLGIPPRFSGSGYGTDFTLTINNIESEDAAYYFCQ |
| | | QYYNLPWTFGQGTKVEIK |
| SEQ ID NO: 153 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAG |
| SEQ ID NO: 154 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAG |
| SEQ ID NO: 155 | Light | DIQMTQSPSSLSASVGDRVTITCSSSQDISNYLNWYQQKPGKAP |
| | chain | KLLIYYTSTLHLGIPPRFSGSGYGTDFTLTINNIESEDAAYYFCQ |
| | | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 156 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAGCGTACGGTGG |
| | | CCGCTCCCAGCGTGTTCATCTTCCCCCCCAGCGACGAGCAGC |
| | | TGAAGAGCGGCACCGCCAGCGTGGTGTGCCTGCTGAACAAC |
| | | TTCTACCCCCGGGAGGCCAAGGTGCAGTGGAAGGTGGACAA |
| | | CGCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCATAAGGTGTACGC |
| | | CTGCGAGGTGACCCACCAGGGCCTGTCCAGCCCCGTGACCA |
| | | AGAGCTTCAACAGGGGCGAGTGC |
| SEQ ID NO: 157 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAGCGTACGGTGGC |
| | | CGCTCCCAGCGTGTTCATCTTCCCCCCAAGCGACGAGCAGCT |
| | | GAAGAGCGGCACCGCCAGCGTGGTGTGTCTGCTGAACAACT |
| | | TCTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone I HC | | |
| SEQ ID NO: 158 | HCDR1 | AATTACGGGATGAAC |
| (Kabat) | | |
| SEQ ID NO: 159 | HCDR1 | AACTACGGCATGAAC |
| (Kabat) | | |
| SEQ ID NO: 160 | HCDR2 | TGGATTAACACCGACACCGGGGAGCCTACCTACGCGGACGA |
| (Kabat) | | TTTCAAGGGA |
| SEQ ID NO: 161 | HCDR2 | TGGATCAACACCGACACCGGCGAGCCTACCTACGCCGACGA |
| (Kabat) | | CTTCAAGGGC |
| SEQ ID NO: 162 | HCDR3 | AACCCGCCCTACTACTACGGAACCAACAACGCCGAAGCCAT |
| (Kabat) | | GGACTAC |
| SEQ ID NO: 163 | HCDR3 | AACCCCCCTTACTACTACGGCACCAACAACGCCGAGGCCAT |
| (Kabat) | | GGACTAT |
| SEQ ID NO: 164 | HCDR1 | GGATTCACCCTCACCAATTAC |
| (Chothia) | | |
| SEQ ID NO: 165 | HCDR1 | GGCTTCACCCTGACCAACTAC |
| (Chothia) | | |
| SEQ ID NO: 166 | HCDR2 | AACACCGACACCGGGGAG |
| (Chothia) | | |
| SEQ ID NO: 167 | HCDR2 | AACACCGACACCGGCGAG |
| (Chothia) | | |
| SEQ ID NO: 162 | HCDR3 | AACCCGCCCTACTACTACGGAACCAACAACGCCGAAGCCAT |
| (Chothia) | | GGACTAC |
| SEQ ID NO: 163) | HCDR3 | AACCCCCCTTACTACTACGGCACCAACAACGCCGAGGCCAT |
| (Chothia) | | GGACTAT |
| BAP050-Clone I LC | | |
| SEQ ID NO: 168 | LCDR1 | AGCTCTAGTCAGGATATCTCTAACTACCTGAAC |
| (Kabat) | | |
| SEQ ID NO: 169 | LCDR1 | TCCTCCAGCCAGGACATCTCCAACTACCTGAAC |
| (Kabat) | | |
| SEQ ID NO: 170 | LCDR2 | TACACTAGCACCCTGCACCTG |
| (Kabat) | | |
| SEQ ID NO: 171 | LCDR2 | TACACCTCCACCCTGCACCTG |
| (Kabat) | | |
| SEQ ID NO: 172 | LCDR3 | CAGCAGTACTATAACCTGCCCTGGACC |
| (Kabat) | | |
| SEQ ID NO: 173 | LCDR3 | CAGCAGTACTACAACCTGCCCTGGACC |
| (Kabat) | | |
| SEQ ID NO: 174 | LCDR1 | AGTCAGGATATCTCTAACTAC |
| (Chothia) | | |
| SEQ ID NO: 175 | LCDR1 | AGCCAGGACATCTCCAACTAC |
| (Chothia) | | |
| SEQ ID NO: 176 | LCDR2 | TACACTAGC |
| (Chothia) | | |
| SEQ ID NO: 177 | LCDR2 | TACACCTCC |
| (Chothia) | | |
| SEQ ID NO: 178 | LCDR3 | TACTATAACCTGCCCTGG |
| (Chothia) | | |
| SEQ ID NO: 179 | LCDR3 | TACTACAACCTGCCCTGG |
| (Chothia) | | |
| BAP050-Clone J HC | | |
| SEQ ID NO: 180 | HCDR1 | AACTACGGGATGAAC |
| (Kabat) | | |
| SEQ ID NO: 159 | HCDR1 | AACTACGGCATGAAC |
| (Kabat) | | |
| SEQ ID NO: 181 | HCDR2 | TGGATTAACACCGACACGGCGAGCCTACCTACGCCGACGA |
| (Kabat) | | CTTTAAGGGC |
| SEQ ID NO: 161 | HCDR2 | TGGATCAACACCGACACCGGCGAGCCTACCTACGCCGACGA |
| (Kabat) | | CTTCAAGGGC |
| SEQ ID NO: 182 | HCDR3 | AACCCCCCCTACTACTACGGCACTAACAACGCCGAGGCTAT |
| (Kabat) | | GGACTAC |
| SEQ ID NO: 163 | HCDR3 | AACCCCCCTTACTACTACGGCACCAACAACGCCGAGGCCAT |
| (Kabat) | | GGACTAT |
| SEQ ID NO: 183 | HCDR1 | GGCTTCACCCTGACTAACTAC |
| (Chothia) | | |
| SEQ ID NO: 165 | HCDR1 | GGCTTCACCCTGACCAACTAC |
| (Chothia) | | |
| SEQ ID NO: 166 | HCDR2 | AACACCGACACCGGGGAG |
| (Chothia) | | |
| SEQ ID NO: 167 | HCDR2 | AACACCGACACCGGCGAG |
| (Chothia) | | |
| SEQ ID NO: 182 | HCDR3 | AACCCCCCCTACTACTACGGCACTAACAACGCCGAGGCTAT |
| (Chothia) | | GGACTAC |
| SEQ ID NO: 163 | HCDR3 | AACCCCCCTTACTACTACGGCACCAACAACGCCGAGGCCAT |
| (Chothia) | | GGACTAT |
| BAP050-Clone J LC | | |
| SEQ ID NO: 168 | LCDR1 | AGCTCTAGTCAGGATATCTCTAACTACCTGAAC |
| (Kabat) | | |
| SEQ ID NO: 169 | LCDR1 | TCCTCCAGCCAGGACATCTCCAACTACCTGAAC |
| (Kabat) | | |
| SEQ ID NO: 170 | LCDR2 | TACACTAGCACCCTGCACCTG |
| (Kabat) | | |
| SEQ ID NO: 171 | LCDR2 | TACACCTCCACCCTGCACCTG |
| (Kabat) | | |
| SEQ ID NO: 172) | LCDR3 | CAGCAGTACTATAACCTGCCCTGGACC |
| (Kabat) | | |
| SEQ ID NO: 173 | LCDR3 | CAGCAGTACTACAACCTGCCCTGGACC |
| (Kabat) | | |
| SEQ ID NO: 174 | LCDR1 | AGTCAGGATATCTCTAACTAC |
| (Chothia) | | |
| SEQ ID NO: 175 | LCDR1 | AGCCAGGACATCTCCAACTAC |
| (Chothia) | | |
| SEQ ID NO: 176 | LCDR2 | TACACTAGC |
| (Chothia) | | |
| SEQ ID NO: 177 | LCDR2 | TACACCTCC |
| (Chothia) | | |
| SEQ ID NO: 178 | LCDR3 | TACTATAACCTGCCCTGG |
| (Chothia) | | |
| SEQ ID NO: 179 | LCDR3 | TACTACAACCTGCCCTGG |
| (Chothia) | | |

### Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 8.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 8. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273.

TABLE 8
Amino acid sequences of other exemplary anti-LAG-3 antibody
molecules

| | | |
|---|---|---|
| BMS-986016 | | |
| SEQ ID NO: 184 | Heavy | QVQLQQWGAGLLKPSETLSLTCAVYGGSFSDYYWNWIRQPPGKG |
| | chain | LEWIGEINHRGSTNSNPSLKSRVTLSLDTSKNQFSLKLRSVTAADTA |
| | | VYYCAFGYSDYEYNWFDPWGQGTLVTVSSASTKGPSVFPLAPCSR |
| | | STSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 185 | Light | EIVLTQSPATLSLSPGERATLSCRASQSISSYLAWYQQKPGQAPRLLI |
| | chain | YDASNRATGIPARFSGSGSGTDFTLTISSLEPEDFAVYYCQQRSNWP |
| | | LTFGQGTNLEIKRTVAAPSVFIFPPSDEQLKSGTASVVCLLNNFYPR |
| | | EAKVQWKVDNALQSGNSQESVTEQDSKDSTYSLSSTLTLSKADYE |
| | | KHKVYACEVTHQGLSSPVTKSFNRGEC |
| IMP731 | | |
| SEQ ID NO: 186 | Heavy | QVQLKESGPGLVAPSQSLSITCTVSGFSLTAYGVNWVRQPPGKGLE |
| | chain | WLGMIWDDGSTDYNSALKSRLSISKDNSKSQVFLKMNSLQTDDTA |
| | | RYYCAREGDVAFDYWGQGTTLTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEY |
| | | KCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID NO: 187 | Light | DIVMTQSPSSLAVSVGQKVTMSCKSSQSLLNGSNQKNYLAWYQQ |
| | chain | KPGQSPKLLVYFASTRDSGVPDRFIGSGSGTDFTLTISSVQAEDLAD |
| | | YFCLQHFGTPPTFGGGTKLEIKRTVAAPSVFIFPPSDEQLKSGTASV |
| | | VCLLNNFYPREAKVQWKVDNALQSGNSQESVTEQDSKDSTYSLSS |
| | | TLTLSKADYEKHKVYACEVTHQGLSSPVTKSFNRGEC |

### TIM-3 Inhibitors

In certain embodiments, the inhibitor of an immune checkpoint molecule is an inhibitor of TIM-3. In some embodiments, TGFβ inhibitors (and/or PD1, PD-L1, or PD-L2 inhibitor) of the present disclosure are used in combination with a TIM-3 inhibitor to treat a disease, e.g., cancer. In some embodiments, the TIM-3 inhibitor is MGB453 (Novartis), LY3321367 (Eli Lilly), Sym023 (Symphogen), BGB-A425 (Beigene), INCAGN-2390 (Agenus/Incyte), MBS-986258 (BMS/Five Prime), RO-7121661 (Roche), LY-3415244 (Eli Lilly), or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof”.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of ABTIM3-hum11 or ABTIM3-hum03 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-TIM-3 antibody molecule comprises a heavy chain variable region (VH) comprising a VHCDR1 amino acid sequence of SEQ ID NO: 189, a VHCDR2 amino acid sequence of SEQ ID NO: 190, and a VHCDR3 amino acid sequence of SEQ ID NO: 191; and a light chain variable region (VL) comprising a VLCDR1 amino acid sequence of SEQ ID NO: 198, a VLCDR2 amino acid sequence of SEQ ID NO: 199, and a VLCDR3 amino acid sequence of SEQ ID NO: 200, each disclosed in Table 9. In one embodiment, the anti-TIM-3 antibody molecule comprises a heavy chain variable region (VH) comprising a VHCDR1 amino acid sequence of SEQ ID NO: 189, a VHCDR2 amino acid sequence of SEQ ID NO: 208, and a VHCDR3 amino acid sequence of SEQ ID NO: 191; and a light chain variable region (VL) comprising a VLCDR1 amino acid sequence of SEQ ID NO: 198, a VLCDR2 amino acid sequence of SEQ ID NO: 199, and a VLCDR3 amino acid sequence of SEQ ID NO: 200, each disclosed in Table 9.

In one embodiment, the anti-TIM-3 antibody molecule comprises a VH comprising the amino acid sequence of SEQ ID NO: 194, or an amino acid sequence at least 85%, 90%, 95%, or 99% identical or higher to SEQ ID NO: 194. In one embodiment, the anti-TIM-3 antibody molecule comprises a VL comprising the amino acid sequence of SEQ ID NO: 204, or an amino acid sequence at least 85%, 90%, 95%, or 99% identical or higher to SEQ ID NO: 204. In one embodiment, the anti-TIM-3 antibody molecule comprises a VH comprising the amino acid sequence of SEQ ID NO: 210, or an amino acid sequence at least 85%, 90%, 95%, or 99% identical or higher to SEQ ID NO: 210. In one embodiment, the anti-TIM-3 antibody molecule comprises a VL comprising the amino acid sequence of SEQ ID NO: 214, or an amino acid sequence at least 85%, 90%, 95%, or 99% identical or higher to SEQ ID NO: 214. In one embodiment, the anti-TIM-3 antibody molecule comprises a VH comprising the amino acid sequence of SEQ ID NO: 194 and a VL comprising the amino acid sequence of SEQ ID NO: 204. In one embodiment, the anti-TIM-3 antibody molecule comprises a VH comprising the amino acid sequence of SEQ ID NO: 210 and a VL comprising the amino acid sequence of SEQ ID NO: 214.

In one embodiment, the antibody molecule comprises a VH encoded by the nucleotide sequence of SEQ ID NO: 195, or a nucleotide sequence at least 85%, 90%, 95%, or 99% identical or higher to SEQ ID NO: 195. In one embodiment, the antibody molecule comprises a VL encoded by the nucleotide sequence of SEQ ID NO: 205, or a nucleotide sequence at least 85%, 90%, 95%, or 99% identical or higher to SEQ ID NO: 205. In one embodiment, the antibody molecule comprises a VH encoded by the nucleotide sequence of SEQ ID NO: 211, or a nucleotide sequence at least 85%, 90%, 95%, or 99% identical or higher to SEQ ID NO: 211. In one embodiment, the antibody molecule comprises a VL encoded by the nucleotide sequence of SEQ ID NO: 215, or a nucleotide sequence at least 85%, 90%, 95%, or 99% identical or higher to SEQ ID NO: 215. In one embodiment, the antibody molecule comprises a VH encoded by the nucleotide sequence of SEQ ID NO: 195 and a VL encoded by the nucleotide sequence of SEQ ID NO: 205. In one embodiment, the antibody molecule comprises a VH encoded by the nucleotide sequence of SEQ ID NO: 211 and a VL encoded by the nucleotide sequence of SEQ ID NO: 215.

In one embodiment, the anti-TIM-3 antibody molecule comprises a heavy chain comprising the amino acid sequence of SEQ ID NO: 196, or an amino acid sequence at least 85%, 90%, 95%, or 99% identical or higher to SEQ ID NO: 196. In one embodiment, the anti-TIM-3 antibody molecule comprises a light chain comprising the amino acid sequence of SEQ ID NO: 206, or an amino acid sequence at least 85%, 90%, 95%, or 99% identical or higher to SEQ ID NO: 206. In one embodiment, the anti-TIM-3 antibody molecule comprises a heavy chain comprising the amino acid sequence of SEQ ID NO: 212, or an amino acid sequence at least 85%, 90%, 95%, or 99% identical or higher to SEQ ID NO: 212. In one embodiment, the anti-TIM-3 antibody molecule comprises a light chain comprising the amino acid sequence of SEQ ID NO: 216, or an amino acid sequence at least 85%, 90%, 95%, or 99% identical or higher to SEQ ID NO: 216. In one embodiment, the anti-TIM-3 antibody molecule comprises a heavy chain comprising the amino acid sequence of SEQ ID NO: 196 and a light chain comprising the amino acid sequence of SEQ ID NO: 206. In one embodiment, the anti-TIM-3 antibody molecule comprises a heavy chain comprising the amino acid sequence of SEQ ID NO: 212 and a light chain comprising the amino acid sequence of SEQ ID NO: 216.

In one embodiment, the antibody molecule comprises a heavy chain encoded by the nucleotide sequence of SEQ ID NO: 197, or a nucleotide sequence at least 85%, 90%, 95%, or 99% identical or higher to SEQ ID NO: 197. In one embodiment, the antibody molecule comprises a light chain encoded by the nucleotide sequence of SEQ ID NO: 207, or a nucleotide sequence at least 85%, 90%, 95%, or 99% identical or higher to SEQ ID NO: 207. In one embodiment, the antibody molecule comprises a heavy chain encoded by the nucleotide sequence of SEQ ID NO: 213, or a nucleotide sequence at least 85%, 90%, 95%, or 99% identical or higher to SEQ ID NO: 213. In one embodiment, the antibody molecule comprises a light chain encoded by the nucleotide sequence of SEQ ID NO: 217, or a nucleotide sequence at least 85%, 90%, 95%, or 99% identical or higher to SEQ ID NO: 217. In one embodiment, the antibody molecule comprises a heavy chain encoded by the nucleotide sequence of SEQ ID NO: 197 and a light chain encoded by the nucleotide sequence of SEQ ID NO: 207. In one embodiment, the antibody molecule comprises a heavy chain encoded by the nucleotide sequence of SEQ ID NO: 213 and a light chain encoded by the nucleotide sequence of SEQ ID NO: 217.

The antibody molecules described herein can be made by vectors, host cells, and methods described in US 2015/0218274.

TABLE 9
Amino acid and nucleotide sequences of exemplary anti-TIM-3
antibody molecules

| | | |
|---|---|---|
| ABTIM3-hum11 | | |
| SEQ ID NO: 189 | HCDR1 | SYNMH |
| (Kabat) | | |
| SEQ ID NO: 190 | HCDR2 | DIYPGNGDTSYNQKFKG |
| (Kabat) | | |
| SEQ ID NO: 191 | HCDR3 | VGGAFPMDY |
| (Kabat) | | |
| SEQ ID NO: 192 | HCDR1 | GYTFTSY |
| (Chothia) | | |
| SEQ ID NO: 193 | HCDR2 | YPGNGD |
| (Chothia) | | |
| SEQ ID NO: 191 | HCDR3 | VGGAFPMDY |
| (Chothia) | | |
| SEQ ID NO: 194 | VH | QVQLVQSGAEVKKPGSSVKVSCKASGYTFTSYNMHWVRQAPGQ |
| | | GLEWMGDIYPGNGDTSYNQKFKGRVTITADKSTSTVYMELSSLR |
| | | SEDTAVYYCARVGGAFPMDYWGQGTTVTVSS |
| SEQ ID NO: 195 | DNA | CAGGTGCAGCTGGTGCAGTCAGGCGCCGAAGTGAAGAAACCC |
| | VH | GGCTCTAGCGTGAAAGTTTCTTGTAAAGCTAGTGGCTACACCT |
| | | TCACTAGCTATAATATGCACTGGGTTCGCCAGGCCCCAGGGCA |
| | | AGGCCTCGAGTGGATGGGCGATATCTACCCCGGGAACGGCGA |
| | | CACTAGTTATAATCAGAAGTTTAAGGGTAGAGTCACTATCACC |
| | | GCCGATAAGTCTACTAGCACCGTCTATATGGAACTGAGTTCCC |
| | | TGAGGTCTGAGGACACCGCCGTCTACTACTGCGCTAGAGTGG |
| | | GCGGAGCCTTCCCTATGGACTACTGGGGTCAAGGCACTACCGT |
| | | GACCGTGTCTAGC |
| SEQ ID NO: 196 | Heavy | QVQLVQSGAEVKKPGSSVKVSCKASGYTFTSYNMHWVRQAPGQ |
| | chain | GLEWMGDIYPGNGDTSYNQKFKGRVTITADKSTSTVYMELSSLR |
| | | SEDTAVYYCARVGGAFPMDYWGQGTTVTVSSASTKGPSVFPLA |
| | | PCSRSTSESTAALGCLVKDYFPEPVTVSWNSGALTSGVHTFPAVL |
| | | QSSGLYSLSSVVTVPSSSLGTKTYTCNVDHKPSNTKVDKRVESKY |
| | | GPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQ |
| | | DWLNGKEYKCKVSNKGLPSSIEKTISKAKGQPREPQVYTLPPSQE |
| | | EMTKNQVSLTCLVKGFYPSDIAVEWESNGQPENNYKTTPPVLDS |
| | | DGSFFLYSRLTVDKSRWQEGNVFSCSVMHEALHNHYTQKSLSLS |
| | | LG |
| SEQ ID NO: 197 | DNA | CAGGTGCAGCTGGTGCAGTCAGGCGCCGAAGTGAAGAAACCC |
| | heavy | GGCTCTAGCGTGAAAGTTTCTTGTAAAGCTAGTGGCTACACCT |
| | chain | TCACTAGCTATAATATGCACTGGGTTCGCCAGGCCCCAGGGCA |
| | | AGGCCTCGAGTGGATGGGCGATATCTACCCCGGGAACGGCGA |
| | | CACTAGTTATAATCAGAAGTTTAAGGGTAGAGTCACTATCACC |
| | | GCCGATAAGTCTACTAGCACCGTCTATATGGAACTGAGTTCCC |
| | | TGAGGTCTGAGGACACCGCCGTCTACTACTGCGCTAGAGTGG |
| | | GCGGAGCCTTCCCTATGGACTACTGGGGTCAAGGCACTACCGT |
| | | GACCGTGTCTAGCGCTAGCACTAAGGGCCCGTCCGTGTTCCCC |
| | | CTGGCACCTTGTAGCCGGAGCACTAGCGAATCCACCGCTGCCC |
| | | TCGGCTGCCTGGTCAAGGATTACTTCCCGGAGCCCGTGACCGT |
| | | GTCCTGGAACAGCGGAGCCCTGACCTCCGGAGTGCACACCTTC |
| | | CCCGCTGTGCTGCAGAGCTCCGGGCTGTACTCGCTGTCGTCGG |
| | | TGGTCACGGTGCCTTCATCTAGCCTGGGTACCAAGACCTACAC |
| | | TTGCAACGTGGACCACAAGCCTTCCAACACTAAGGTGGACAA |
| | | GCGCGTCGAATCGAAGTACGGCCCACCGTGCCCGCCTTGTCCC |
| | | GCGCCGGAGTTCCTCGGCGGTCCCTCGGTCTTTCTGTTCCCAC |
| | | CGAAGCCCAAGGACACTTTGATGATTTCCCGCACCCCTGAAGT |
| | | GACATGCGTGGTCGTGGACGTGTCACAGGAAGATCCGGAGGT |
| | | GCAGTTCAATTGGTACGTGGATGGCGTCGAGGTGCACAACGC |
| | | CAAAACCAAGCCGAGGGAGGAGCAGTTCAACTCCACTTACCG |
| | | CGTCGTGTCCGTGCTGACGGTGCTGCATCAGGACTGGCTGAAC |
| | | GGGAAGGAGTACAAGTGCAAAGTGTCCAACAAGGGACTTCCT |
| | | AGCTCAATCGAAAAGACCATCTCGAAAGCCAAGGGACAGCCC |
| | | CGGGAACCCCAAGTGTATACCCTGCCACCGAGCCAGGAAGAA |
| | | ATGACTAAGAACCAAGTCTCATTGACTTGCCTTGTGAAGGGCT |
| | | TCTACCCATCGGATATCGCCGTGGAATGGGAGTCCAACGGCC |
| | | AGCCGGAAAACAACTACAAGACCACCCCTCCGGTGCTGGACT |
| | | CAGACGGATCCTTCTTCCTCTACTCGCGGCTGACCGTGGATAA |
| | | GAGCAGATGGCAGGAGGGAAATGTGTTCAGCTGTTCTGTGAT |
| | | GCATGAAGCCCTGCACAACCACTACACTCAGAAGTCCCTGTCC |
| | | CTCTCCCTGGGA |
| SEQ ID NO: 198 | LCDR1 | RASESVEYYGTSLMQ |
| (Kabat) | | |
| SEQ ID NO: 199 | LCDR2 | AASNVES |
| (Kabat) | | |
| SEQ ID NO: 200 | LCDR3 | QQSRKDPST |
| (Kabat) | | |
| SEQ ID NO: 201 | LCDR1 | SESVEYYGTSL |
| (Chothia) | | |
| SEQ ID NO: 202 | LCDR2 | AAS |
| (Chothia) | | |
| SEQ ID NO: 203 | LCDR3 | SRKDPS |
| (Chothia) | | |
| SEQ ID NO: 204 | VL | AIQLTQSPSSLSASVGDRVTITCRASESVEYYGTSLMQWYQQKPG |
| | | KAPKLLIYAASNVESGVPSRFSGSGSGTDFTLTISSLQPEDFATYF |
| | | CQQSRKDPSTFGGGTKVEIK |
| SEQ ID NO: 205 | DNA | GCTATTCAGCTGACTCAGTCACCTAGTAGCCTGAGCGCTAGTG |
| | VL | TGGGCGATAGAGTGACTATCACCTGTAGAGCTAGTGAATCAG |
| | | TCGAGTACTACGGCACTAGCCTGATGCAGTGGTATCAGCAGA |
| | | AGCCCGGGAAAGCCCCTAAGCTGCTGATCTACGCCGCCTCTAA |
| | | CGTGGAATCAGGCGTGCCCTCTAGGTTTAGCGGTAGCGGTAGT |
| | | GGCACCGACTTCACCCTGACTATCTCTAGCCTGCAGCCCGAGG |
| | | ACTTCGCTACCTACTTCTGTCAGCAGTCTAGGAAGGACCCTAG |
| | | CACCTTCGGCGGAGGCACTAAGGTCGAGATTAAG |
| SEQ ID NO: 206 | Light | AIQLTQSPSSLSASVGDRVTITCRASESVEYYGTSLMQWYQQKPG |
| | chain | KAPKLLIYAASNVESGVPSRFSGSGSGTDFTLTISSLQPEDFATYF |
| | | CQQSRKDPSTFGGGTKVEIKRTVAAPSVFIFPPSDEQLKSGTASVV |
| | | CLLNNFYPREAKVQWKVDNALQSGNSQESVTEQDSKDSTYSLSS |
| | | TLTLSKADYEKHKVYACEVTHQGLSSPVTKSFNRGEC |
| SEQ ID NO: 207 | DNA | GCTATTCAGCTGACTCAGTCACCTAGTAGCCTGAGCGCTAGTG |
| | light | TGGGCGATAGAGTGACTATCACCTGTAGAGCTAGTGAATCAG |
| | chain | TCGAGTACTACGGCACTAGCCTGATGCAGTGGTATCAGCAGA |
| | | AGCCCGGGAAAGCCCCTAAGCTGCTGATCTACGCCGCCTCTAA |
| | | CGTGGAATCAGGCGTGCCCTCTAGGTTTAGCGGTAGCGGTAGT |
| | | GGCACCGACTTCACCCTGACTATCTCTAGCCTGCAGCCCGAGG |
| | | ACTTCGCTACCTACTTCTGTCAGCAGTCTAGGAAGGACCCTAG |
| | | CACCTTCGGCGGAGGCACTAAGGTCGAGATTAAGCGTACGGT |
| | | GGCCGCTCCCAGCGTGTTCATCTTCCCCCCCAGCGACGAGCAG |
| | | CTGAAGAGCGGCACCGCCAGCGTGGTGTGCCTGCTGAACAAC |
| | | GCCCTGCAGAGCGGCAACAGCCAGGAGAGCGTCACCGAGCAG |
| | | GACAGCAAGGACTCCACCTACAGCCTGAGCAGCACCCTGACC |
| | | CTGAGCAAGGCCGACTACGAGAAGCATAAGGTGTACGCCTGC |
| | | GAGGTGACCCACCAGGGCCTGTCCAGCCCCGTGACCAAGAGC |
| | | TTCAACAGGGGCGAGTGC |
| ABTIM3-hum03 | | |
| SEQ ID NO: 189 | HCDR1 | SYNMH |
| (Kabat) | | |
| SEQ ID NO: 208 | HCDR2 | DIYPGQGDTSYNQKFKG |
| (Kabat) | | |
| SEQ ID NO: 191 | HCDR3 | VGGAFPMDY |
| (Kabat) | | |
| SEQ ID NO: 192 | HCDR1 | GYTFTSY |
| (Chothia) | | |
| SEQ ID NO: 209 | HCDR2 | YPGQGD |
| (Chothia) | | |
| SEQ ID NO: 191 | HCDR3 | VGGAFPMDY |
| (Chothia) | | |
| SEQ ID NO: 210 | VH | QVQLVQSGAEVKKPGASVKVSCKASGYTFTSYNMHWVRQAPG |
| | | QGLEWIGDIYPGQGDTSYNQKFKGRATMTADKSTSTVYMELSSL |
| | | RSEDTAVYYCARVGGAFPMDYWGQGTLVTVSS |
| SEQ ID NO: 211 | DNA | CAGGTGCAGCTGGTGCAGTCAGGCGCCGAAGTGAAGAAACCC |
| | VH | GGCGCTAGTGTGAAAGTTAGCTGTAAAGCTAGTGGCTATACTT |
| | | TCACTTCTTATAATATGCACTGGGTCCGCCAGGCCCCAGGTCA |
| | | AGGCCTCGAGTGGATCGGCGATATCTACCCCGGTCAAGGCGA |
| | | CACTTCCTATAATCAGAAGTTTAAGGGTAGAGCTACTATGACC |
| | | GCCGATAAGTCTACTTCTACCGTCTATATGGAACTGAGTTCCC |
| | | TGAGGTCTGAGGACACCGCCGTCTACTACTGCGCTAGAGTGG |
| | | GCGGAGCCTTCCCAATGGACTACTGGGGTCAAGGCACCCTGG |
| | | TCACCGTGTCTAGC |
| SEQ ID NO: 212 | Heavy | QVQLVQSGAEVKKPGASVKVSCKASGYTFTSYNMHWVRQAPG |
| | chain | QGLEWIGDIYPGQGDTSYNQKFKGRATMTADKSTSTVYMELSSL |
| | | RSEDTAVYYCARVGGAFPMDYWGQGTLVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 213 | DNA | CAGGTGCAGCTGGTGCAGTCAGGCGCCGAAGTGAAGAAACCC |
| | heavy | GGCGCTAGTGTGAAAGTTAGCTGTAAAGCTAGTGGCTATACTT |
| | chain | TCACTTCTTATAATATGCACTGGGTCCGCCAGGCCCCAGGTCA |
| | | AGGCCTCGAGTGGATCGGCGATATCTACCCCGGTCAAGGCGA |
| | | CACTTCCTATAATCAGAAGTTTAAGGGTAGAGCTACTATGACC |
| | | GCCGATAAGTCTACTTCTACCGTCTATATGGAACTGAGTTCCC |
| | | TGAGGTCTGAGGACACCGCCGTCTACTACTGCGCTAGAGTGG |
| | | GCGGAGCCTTCCCAATGGACTACTGGGGTCAAGGCACCCTGG |
| | | TCACCGTGTCTAGCGCTAGCACTAAGGGCCCGTCCGTGTTCCC |
| | | CCTGGCACCTTGTAGCCGGAGCACTAGCGAATCCACCGCTGCC |
| | | CTCGGCTGCCTGGTCAAGGATTACTTCCCGGAGCCCGTGACCG |
| | | TGTCCTGGAACAGCGGAGCCCTGACCTCCGGAGTGCACACCTT |
| | | CCCCGCTGTGCTGCAGAGCTCCGGGCTGTACTCGCTGTCGTCG |
| | | GTGGTCACGGTGCCTTCATCTAGCCTGGGTACCAAGACCTACA |
| | | CTTGCAACGTGGACCACAAGCCTTCCAACACTAAGGTGGACA |
| | | AGCGCGTCGAATCGAAGTACGGCCCACCGTGCCCGCCTTGTCC |
| | | CGCGCCGGAGTTCCTCGGCGGTCCCTCGGTCTTTCTGTTCCCA |
| | | CCGAAGCCCAAGGACACTTTGATGATTTCCCGCACCCCTGAAG |
| | | TGACATGCGTGGTCGTGGACGTGTCACAGGAAGATCCGGAGG |
| | | TGCAGTTCAATTGGTACGTGGATGGCGTCGAGGTGCACAACG |
| | | CCAAAACCAAGCCGAGGGAGGAGCAGTTCAACTCCACTTACC |
| | | GCGTCGTGTCCGTGCTGACGGTGCTGCATCAGGACTGGCTGAA |
| | | CGGGAAGGAGTACAAGTGCAAAGTGTCCAACAAGGGACTTCC |
| | | TAGCTCAATCGAAAAGACCATCTCGAAAGCCAAGGGACAGCC |
| | | CCGGGAACCCCAAGTGTATACCCTGCCACCGAGCCAGGAAGA |
| | | AATGACTAAGAACCAAGTCTCATTGACTTGCCTTGTGAAGGGC |
| | | TTCTACCCATCGGATATCGCCGTGGAATGGGAGTCCAACGGCC |
| | | AGCCGGAAAACAACTACAAGACCACCCCTCCGGTGCTGGACT |
| | | CAGACGGATCCTTCTTCCTCTACTCGCGGCTGACCGTGGATAA |
| | | GAGCAGATGGCAGGAGGGAAATGTGTTCAGCTGTTCTGTGAT |
| | | GCATGAAGCCCTGCACAACCACTACACTCAGAAGTCCCTGTCC |
| | | CTCTCCCTGGGA |
| SEQ ID NO: 198 | LCDR1 | RASESVEYYGTSLMQ |
| (Kabat) | | |
| SEQ ID NO: 199 | LCDR2 | AASNVES |
| (Kabat) | | |
| SEQ ID NO: 200 | LCDR3 | QQSRKDPST |
| (Kabat) | | |
| SEQ ID NO: 201 | LCDR1 | SESVEYYGTSL |
| (Chothia) | | |
| SEQ ID NO: 202 | LCDR2 | AAS |
| (Chothia) | | |
| SEQ ID NO: 203 | LCDR3 | SRKDPS |
| (Chothia) | | |
| SEQ ID NO: 214 | VL | DIVLTQSPDSLAVSLGERATINCRASESVEYYGTSLMQWYQQKP |
| | | GQPPKLLIYAASNVESGVPDRFSGSGSGTDFTLTISSLQAEDVAVY |
| | | YCQQSRKDPSTFGGGTKVEIK |
| SEQ ID NO: 215 | DNA | GATATCGTCCTGACTCAGTCACCCGATAGCCTGGCCGTCAGCC |
| | VL | TGGGCGAGCGGGCTACTATTAACTGTAGAGCTAGTGAATCAG |
| | | TCGAGTACTACGGCACTAGCCTGATGCAGTGGTATCAGCAGA |
| | | AGCCCGGTCAACCCCCTAAGCTGCTGATCTACGCCGCCTCTAA |
| | | CGTGGAATCAGGCGTGCCCGATAGGTTTAGCGGTAGCGGTAG |
| | | TGGCACCGACTTCACCCTGACTATTAGTAGCCTGCAGGCCGAG |
| | | GACGTGGCCGTCTACTACTGTCAGCAGTCTAGGAAGGACCCTA |
| | | GCACCTTCGGCGGAGGCACTAAGGTCGAGATTAAG |
| SEQ ID NO: 216 | Light | DIVLTQSPDSLAVSLGERATINCRASESVEYYGTSLMQWYQQKP |
| | chain | GQPPKLLIYAASNVESGVPDRFSGSGSGTDFTLTISSLQAEDVAVY |
| | | YCQQSRKDPSTFGGGTKVEIKRTVAAPSVFIFPPSDEQLKSGTASV |
| | | VCLLNNFYPREAKVQWKVDNALQSGNSQESVTEQDSKDSTYSLS |
| | | STLTLSKADYEKHKVYACEVTHQGLSSPVTKSFNRGEC |
| SEQ ID NO: 217 | DNA | GATATCGTCCTGACTCAGTCACCCGATAGCCTGGCCGTCAGCC |
| | light | TGGGCGAGCGGGCTACTATTAACTGTAGAGCTAGTGAATCAG |
| | chain | TCGAGTACTACGGCACTAGCCTGATGCAGTGGTATCAGCAGA |
| | | AGCCCGGTCAACCCCCTAAGCTGCTGATCTACGCCGCCTCTAA |
| | | CGTGGAATCAGGCGTGCCCGATAGGTTTAGCGGTAGCGGTAG |
| | | TGGCACCGACTTCACCCTGACTATTAGTAGCCTGCAGGCCGAG |
| | | GACGTGGCCGTCTACTACTGTCAGCAGTCTAGGAAGGACCCTA |
| | | GCACCTTCGGCGGAGGCACTAAGGTCGAGATTAAGCGTACGG |
| | | TGGCCGCTCCCAGCGTGTTCATCTTCCCCCCCAGCGACGAGCA |
| | | GCTGAAGAGCGGCACCGCCAGCGTGGTGTGCCTGCTGAACAA |
| | | CTTCTACCCCCGGGAGGCCAAGGTGCAGTGGAAGGTGGACAA |
| | | CGCCCTGCAGAGCGGCAACAGCCAGGAGAGCGTCACCGAGCA |
| | | GGACAGCAAGGACTCCACCTACAGCCTGAGCAGCACCCTGAC |
| | | CCTGAGCAAGGCCGACTACGAGAAGCATAAGGTGTACGCCTG |
| | | CGAGGTGACCCACCAGGGCCTGTCCAGCCCCGTGACCAAGAG |
| | | CTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 10. APE5137, APE5121, and other anti-TIM-3 antibodies are disclosed in WO 2016/161270.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

In one embodiment, the anti-TIM-3 antibody molecule is LY3321367 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of LY3321367.

In one embodiment, the anti-TIM-3 antibody molecule is Sym023 (Symphogen). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of Sym023.

In one embodiment, the anti-TIM-3 antibody molecule is BGB-A425 (Beigene). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BGB-A425.

In one embodiment, the anti-TIM-3 antibody molecule is INCAGN-2390 (Agenus/Incyte). In one embodiment, the anti-TIM-3 antibody molecule comprises one or more of the CDR sequences (or collectively all of the CDR sequences), the heavy chain variable region sequence and/or light chain variable region sequence, or the heavy chain or light chain sequence of INCAGN-2390.

In one embodiment, the anti-TIM-3 antibody molecule is BMS-986258 (BMS/Five Prime). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of BMS-986258.

In one embodiment, the anti-TIM-3 antibody or inhibitor molecule is RO-7121661 (Roche). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of the TIM-3 binding arm of RO-7121661.

In one embodiment, the anti-TIM-3 antibody or inhibitor molecule is LY-3415244 (Eli Lilly). In one embodiment, the anti-TIM-3 antibody molecule comprises one or more of the CDR sequences (or collectively all of the CDR sequences), the heavy chain variable region sequence and/or light chain variable region sequence, or the heavy chain sequence and/or light chain sequence of the TIM-3 binding arm of LY-3415244.

Further known anti-TIM-3 antibodies include those described, e.g., in WO 2016/111947, WO 2016/071448, WO 2016/144803, U.S. Pat. Nos. 8,552,156, 8,841,418, and 9,163,087.

In one embodiment, the anti-TIM-3 antibody is an antibody that competes for binding with, and/or binds to the same epitope on TIM-3 as, one of the anti-TIM-3 antibodies described herein.

TABLE 10
Amino acid sequences of other exemplary
anti-TIM-3 antibody molecules

| | | |
|---|---|---|
| APE5137 | | |
| SEQ ID NO: 218 | VH | EVQLLESGGGLVQPGGSLRLSCAAASGFTF |
| | | SSYDMSWVRQAPGKGLDWVSTISGGGTYTY |
| | | YQDSVKGRFTISRDNSKNTLYLQMNSLRAE |
| | | DTAVYYCASMDYWGQGTTVTVSSA |
| SEQ ID NO: 219 | VL | DIQMTQSPSSLSASVGDRVTITCRASQSIR |
| | | YRYLNWYHQKPGKAPKLLIGASTLQSGVPS |
| | | RFSGSGSGTDFTLTISSLQPEDFAVYYCQQ |
| | | SHSAPLTFGGGTKVEIKR |
| APE5121 | | |
| SEQ ID NO: 220 | VH | EVQVLESGGGLVQPGGSLRLYCVASGFTFS |
| | | GSYAMSWVRQAPGKGLEWVSAISGSGGSTY |
| | | YADSVKGRFTISRDNSKNTLYLQMNSLRAE |
| | | DTAVYYCAKKYYVGPADYWGQGTLVTVSSG |
| SEQ ID NO: 221 | VL | DIVMTQSPDSLAVSLGERATINCKSSQSVL |
| | | YSSNNKNYLAWYQHKPGQPPKLLIYWASTR |
| | | ESGVPDRFSGSGSGTDFTLTISSLQAEDVA |
| | | VYYCQQYYSSPLTFGGGTKIEVK |

### Cytokines

In yet another embodiment, TGFβ inhibitors (and/or PD1, PD-L1, or PD-L2 inhibitor), of the present disclosure are used in combination with one or more cytokines, including but not limited to, interferon, IL-2, IL-15, IL-7, or IL21. In certain embodiments, the TGFβ inhibitors (and/or PD1, PD-L1, or PD-L2 inhibitor) are administered in combination with an IL-15/IL-15Ra complex. In some embodiments, the IL-15/IL-15Ra complex is selected from NIZ985 (Novartis), ATL-803 (Altor) or CYP0150 (Cytune).

### Exemplary IL-15/IL-15Ra Complexes

In one embodiment, the cytokine is IL-15 complexed with a soluble form of IL-15 receptor alpha (IL-15Ra). The IL-15/IL-15Ra complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the formulation comprises an amino acid sequence of SEQ ID NO: 222 in Table 11 or an amino acid sequence at least 85%, 90%, 95%, or 99% identical or higher to SEQ ID NO: 222, and the soluble form of human IL-15Ra comprises an amino acid sequence of SEQ ID NO: 223 in Table 11, or an amino acid sequence at least 85%, 90%, 95%, or 99% identical or higher to SEQ ID NO: 223, as described in WO 2014/066527. The molecules described herein can be made by vectors, host cells, and methods described in WO 2007084342.

TABLE 11
Amino acid and nucleotide sequences of
exemplary IL-15/IL-15Ra complexes

| | | |
|---|---|---|
| NIZ985 | | |
| SEQ ID NO: 222 | Human | NWVNVISDLKKIEDLIQSMHIDATLYT |
| | IL-15 | ESDVHPSCKVTAMKCFLLELQVISLES |
| | | GDASIHDTVENLIILANNSLSSNGNVT |
| | | ESGCKECEELEEKNIKEFLQSFVHIVQ |
| | | MFINTS |
| SEQ ID NO: 223 | Human | ITCPPPMSVEHADIWVKSYSLYSRERY |
| | Soluble | ICNSGFKRKAGTSSLTECVLNKATNVA |
| | IL-15Ra | HWTTPSLKCIRDPALVHQRPAPPSTVT |
| | | TAGVTPQPESLSPSGKEPAASSPSSNN |
| | | TAATTAAIVPGSQLMPSKSPSTGTTEI |
| | | SSHESSHGTPSQTTAKNWELTASASHQ |
| | | PPGVYPQG |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is described in WO 2008/143794. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is described in WO 2007/04606 and WO 2012/175222. In one embodiment, the IL-15/IL-15Ra sushi domain fusion comprises the sequences as disclosed in Table 12.

Amino acid sequences of other exemplary
IL-15/IL-15Ra complexes
ALT-803

| | | |
|---|---|---|
| SEQ ID NO: | IL-15N72D | NWVNVISDLKKIEDLIQSMH |
| 224 | | IDATLYTESDVHPSCKVTAM |
| | | KCFLLELQVISLESGDASIH |
| | | DTVENLIILANDSLSSNGNV |
| | | TESGCKECEELEEKNIKEFL |
| | | QSFVHIVQMFINTS |
| SEQ ID NO: | IL- | ITCPPPMSVEHADIWVKSYS |
| 225 | 15RaSu/Fc | LYSRERYICNSGFKRKAGTS |
| | | SLTECVLNKATNVAHWTTPS |
| | | LKCIREPKSCDKTHTCPPCP |
| | | APELLGGPSVFLFPPKPKDT |
| | | LMISRTPEVTCVVVDVSHED |
| | | PEVKFNWYVDGVEVHNAKTK |
| | | PREEQYNSTYRVVSVLTVLH |
| | | QDWLNGKEYKCKVSNKALPA |
| | | PIEKTISKAKGQPREPQVYT |
| IL-15/ | a sushi | LPPSRDELTKNQVSLTCLVK |
| IL-151 | domain | GFYPSDIAVEWESNGQPENN |
| | | YKTTPPVLDSDGSFFLYSKL |
| | | TVDKSRWQQGNVFSCSVMHE |
| | | ALHNHYTQKSLSLSPGK |

IL-15/IL-15Ra sushi domain fusion (CYP0150)

| | | |
|---|---|---|
| SEQ ID NO: | Human | NWVNVISDLKKIEDLIQSMH |
| 226 | IL-15 | IDATLYTESDVHPSCKVTAM |
| | | KCFLLELQVISLESGDASIH |
| | | DTVENLIILANNSLSSNGNV |
| | | TESGCKECEELEXKNIKEFL |
| | | QSFVHIVQMFINTS |
| | | Where X is E or K |
| SEQ ID NO: | Human IL- | ITCPPPMSVEHADIWVKSYS |
| 227 | 15Ra sushi | LYSRERYICNSGFKRKAGTS |
| | and hinge | SLTECVLNKATNVAHWTTPS |
| | domains | LKCIRDPALVHQRPAPP |

In yet another embodiment, TGFβ inhibitors (and/or PD1, PD-L1, or PD-L2 inhibitor) of the present disclosure are used in combination with one or more agonists of toll like receptors (TLRs, e.g., TLR7, TLR8, TLR9) to treat a disease, e.g., cancer. In some embodiments, a compound of the present disclosure can be used in combination with a TLR7 agonist or a TLR7 agonist conjugate.

In some embodiments, the TLR7 agonist comprises a compound disclosed in International Application Publication No. WO2011/049677. In some embodiments, the TLR7 agonist comprises 3-(5-amino-2-(4-(2-(3,3-difluoro-3-phosphonopropoxy)ethoxy)methylphenethyl)benzo[1,7]naphthyridin-8-yl)propanoic acid. In some embodiments, the TLR7 agonist comprises a compound of formula:

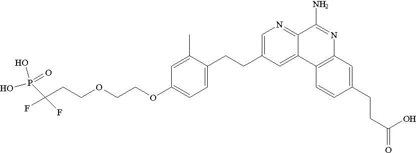 [SMILES: Cc1cc(OCCOCCC(F)(F)P(=O)(O)O)ccc1CCc1cnc2c(N)nc3cc(CCC(=O)O)ccc3c2c1]

In another embodiment, TGFβ inhibitors (and/or PD1, PD-L1, or PD-L2 inhibitor) of the present disclosure are used in combination with one or more angiogenesis inhibitors to treat cancer, e.g., Bevacizumab (Avastin®), axitinib (Inlyta®); Brivanib alaninate (BMS-582664, (S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, TGFβ inhibitors (and/or PD1, PD-L1, or PD-L2 inhibitor) of the present disclosure are used in combination with one or more heat shock protein inhibitors to treat cancer, e.g., Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (BIIB021 or -CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In yet another embodiment, TGFβ inhibitors (and/or PD1, PD-L1, or PD-L2 inhibitor) of the present disclosure are used in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cyl-1); Cyclo[(αS,2S)-α-amino-η-oxo-2-oxiraneoctanoyl-O-methyl-D-tyrosyl-L-isoleucyl-(2S)-2-piperidinecarbonyl] (Cyl-2); Cyclic[L-alanyl-D-alanyl-(2S)-η-oxo-L-α-aminooxiraneoctanoyl-D-prolyl] (HC-toxin); Cyclo[(αS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat (Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase 1A or KDM1A).

In yet another embodiment, TGFβ inhibitors (and/or PD1, PD-L1, or PD-L2 inhibitor) of the present disclosure are used in combination with one or more inhibitors of indoleamine-pyrrole 2,3-dioxygenase (IDO), for example, Indoximod (also known as NLG-8189), α-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360), to treat cancer.

### Chimeric Antigen Receptors

The present disclosure provides for TGFβ inhibitors (and/or PD1, PD-L1, or PD-L2 inhibitor) for use in combination with adoptive immunotherapy methods and reagents such as chimeric antigen receptor (CAR) immune effector cells, e.g., T cells, or chimeric TCR-transduced immune effector cells, e.g., T cells. This section describes CAR technology generally that is useful in combination with TGFβ inhibitors (and/or PD1, PD-L1, or PD-L2 inhibitor), and describes CAR reagents, e.g., cells and compositions, and methods.

In general, aspects of the present disclosure pertain to or include an isolated nucleic acid molecule encoding a chimeric antigen receptor (CAR), wherein the CAR comprises an antigen binding domain (e.g., antibody or antibody fragment, TCR or TCR fragment) that binds to a tumor antigen as described herein, a transmembrane domain (e.g., a transmembrane domain described herein), and an intracellular signalling domain (e.g., an intracellular signalling domain described herein) (e.g., an intracellular signalling domain comprising a costimulatory domain (e.g., a costimulatory domain described herein) and/or a primary signalling domain (e.g., a primary signalling domain described herein). In other aspects, the present disclosure includes: host cells containing the above nucleic acids and isolated proteins encoded by such nucleic acid molecules. CAR nucleic acid constructs, encoded proteins, containing vectors, host cells, pharmaceutical compositions, and methods of administration and treatment related to the present disclosure are disclosed in detail in International Patent Application Publication No. WO2015142675.

In one aspect, the disclosure pertains to an isolated nucleic acid molecule encoding a chimeric antigen receptor (CAR), wherein the CAR comprises an antigen binding domain (e.g., antibody or antibody fragment, TCR or TCR fragment) that binds to a tumor-supporting antigen (e.g., a tumor-supporting antigen as described herein), a transmembrane domain (e.g., a transmembrane domain described herein), and an intracellular signalling domain (e.g., an intracellular signalling domain described herein) (e.g., an intracellular signalling domain comprising a costimulatory domain (e.g., a costimulatory domain described herein) and/or a primary signalling domain (e.g., a primary signalling domain described herein). In some embodiments, the tumor-supporting antigen is an antigen present on a stromal cell or a myeloid-derived suppressor cell (MDSC). In other aspects, the disclosure features polypeptides encoded by such nucleic acids and host cells containing such nucleic acids and/or polypeptides.

Alternatively, aspects of the disclosure pertain to isolated nucleic acid encoding a chimeric T cell receptor (TCR) comprising a TCR alpha and/or TCR beta variable domain with specificity for a cancer antigen described herein. See for example, Dembic et al., Nature, 320, 232-238 (1986), Schumacher, Nat. Rev. Immunol., 2, 512-519 (2002), Kershaw et al., Nat. Rev. Immunol., 5, 928-940 (2005), Xue et al., Clin. Exp. Immunol., 139, 167-172 (2005), Rossig et al., Mol. Ther., 10, 5-18 (2004), and Murphy et al., Immunity, 22, 403-414 (2005); (Morgan et al. J. Immunol., 171, 3287-3295 (2003), Hughes et al., Hum. Gene Ther., 16, 1-16 (2005), Zhao et al., J. Immunol., 174, 4415-4423 (2005), Roszkowski et al., Cancer Res., 65, 1570-1576 (2005), and Engels et al., Hum. Gene Ther., 16, 799-810 (2005); US2009/03046557. Such chimeric TCRs may recognize, for example, cancer antigens such as MART-1, -100, p53, and NY-ESO-1, MAGE A3/A6, MAGEA3, SSX2, HPV-16 E6 or HPV-16 E7. In other aspects, the disclosure features polypeptides encoded by such nucleic acids and host cells containing such nucleic acids and/or polypeptides.

Sequences of non-limiting examples of various components that can be part of a CAR are listed in Table 11a, where “aa” stands for amino acids, and “na” stands for nucleic acids that encode the corresponding peptide.

TABLE lla
Sequences of various components of CAR
(aa-amino acid sequence, na-nucleic acid sequence).

| SEQ ID | | |
|---|---|---|
| NO: | description | Sequence |
| SEQ ID | EF-1 | CGTGAGGCTCCGGTGCCCGTCAGTGGGCAGAGCGCACATCGC |
| NO: 285 | promoter | CCACAGTCCCCGAGAAGTTGGGGGGAGGGGTCGGCAATTGAA |
| | (na) | CCGGTGCCTAGAGAAGGTGGCGCGGGGTAAACTGGGAAAGTG |
| | | ATGTCGTGTACTGGCTCCGCCTTTTTCCCGAGGGTGGGGGAGA |
| | | ACCGTATATAAGTGCAGTAGTCGCCGTGAACGTTCTTTTTCGC |
| | | AACGGGTTTGCCGCCAGAACACAGGTAAGTGCCGTGTGTGGT |
| | | TCCCGCGGGCCTGGCCTCTTTACGGGTTATGGCCCTTGCGTGC |
| | | CTTGAATTACTTCCACCTGGCTGCAGTACGTGATTCTTGATCCC |
| | | GAGCTTCGGGTTGGAAGTGGGTGGGAGAGTTCGAGGCCTTGC |
| | | GCTTAAGGAGCCCCTTCGCCTCGTGCTTGAGTTGAGGCCTGGC |
| | | CTGGGCGCTGGGGCCGCCGCGTGCGAATCTGGTGGCACCTTCG |
| | | CGCCTGTCTCGCTGCTTTCGATAAGTCTCTAGCCATTTAAAATT |
| | | TTTGATGACCTGCTGCGACGCTTTTTTTCTGGCAAGATAGTCTT |
| | | GTAAATGCGGGCCAAGATCTGCACACTGGTATTTCGGTTTTTG |
| | | GGGCCGCGGGCGGCGACGGGGCCCGTGCGTCCCAGCGCACAT |
| | | GTTCGGCGAGGCGGGGCCTGCGAGCGCGGCCACCGAGAATCG |
| | | GACGGGGGTAGTCTCAAGCTGGCCGGCCTGCTCTGGTGCCTGG |
| | | CCTCGCGCCGCCGTGTATCGCCCCGCCCTGGGCGGCAAGGCTG |
| | | GCCCGGTCGGCACCAGTTGCGTGAGCGGAAAGATGGCCGCTT |
| | | CCCGGCCCTGCTGCAGGGAGCTCAAAATGGAGGACGCGGCGC |
| | | TCGGGAGAGCGGGCGGGTGAGTCACCCACACAAAGGAAAAG |
| | | GGCCTTTCCGTCCTCAGCCGTCGCTTCATGTGACTCCACGGAG |
| | | TACCGGGCGCCGTCCAGGCACCTCGATTAGTTCTCGAGCTTTT |
| | | GGAGTACGTCGTCTTTAGGTTGGGGGGAGGGGTTTTATGCGAT |
| | | GGAGTTTCCCCACACTGAGTGGGTGGAGACTGAAGTTAGGCC |
| | | AGCTTGGCACTTGATGTAATTCTCCTTGGAATTTGCCCTTTTTG |
| | | AGTTTGGATCTTGGTTCATTCTCAAGCCTCAGACAGTGGTTCA |
| | | AAGTTTTTTTCTTCCATTTCAGGTGTCGTGA |
| SEQ ID | Leader (aa) | MALPVTALLLPLALLLHAARP |
| NO: 283 | | |
| SEQ ID | Leader (na) | ATGGCCCTGCCTGTGACAGCCCTGCTGCTGCCTCTGGCTCTGC |
| NO: | | TGCTGCATGCCGCTAGACCC |
| 299 | | |
| SEQ ID | Leader (na) | ATGGCCCTCCCTGTCACCGCCCTGCTGCTTCCGCTGGCTCTTCT |
| NO: | | GCTCCACGCCGCTCGGCCC |
| 300 | | |
| SEQ ID | CD 8 hinge | TTTPAPRPPTPAPTIASQPLSLRPEACRPAAGGAVHTRGLDFACD |
| NO: 265 | (aa) | |
| SEQ ID | CD8 hinge | ACCACGACGCCAGCGCCGCGACCACCAACACCGGCGCCCACC |
| NO: 269 | (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGAT |
| SEQ ID | IgG4 hinge | ESKYGPPCPPCPAPEFLGGPSVFLFPPKPKDTLMISRTPEVTCVV |
| NO: 268 | (aa) | VDVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQK |
| | | SLSLSLGKM |
| SEQ ID | IgG4 hinge | GAGAGCAAGTACGGCCCTCCCTGCCCCCCTTGCCCTGCCCCCG |
| NO: 270 | (na) | AGTTCCTGGGCGGACCCAGCGTGTTCCTGTTCCCCCCCAAGCC |
| | | CAAGGACACCCTGATGATCAGCCGGACCCCCGAGGTGACCTG |
| | | TGTGGTGGTGGACGTGTCCCAGGAGGACCCCGAGGTCCAGTT |
| | | CAACTGGTACGTGGACGGCGTGGAGGTGCACAACGCCAAGAC |
| | | CAAGCCCCGGGAGGAGCAGTTCAATAGCACCTACCGGGTGGT |
| | | GTCCGTGCTGACCGTGCTGCACCAGGACTGGCTGAACGGCAA |
| | | GGAATACAAGTGTAAGGTGTCCAACAAGGGCCTGCCCAGCAG |
| | | CATCGAGAAAACCATCAGCAAGGCCAAGGGCCAGCCTCGGGA |
| | | GCCCCAGGTGTACACCCTGCCCCCTAGCCAAGAGGAGATGAC |
| | | CAAGAACCAGGTGTCCCTGACCTGCCTGGTGAAGGGCTTCTAC |
| | | CCCAGCGACATCGCCGTGGAGTGGGAGAGCAACGGCCAGCCC |
| | | GAGAACAACTACAAGACCACCCCCCCTGTGCTGGACAGCGAC |
| | | GGCAGCTTCTTCCTGTACAGCCGGCTGACCGTGGACAAGAGCC |
| | | GGTGGCAGGAGGGCAACGTCTTTAGCTGCTCCGTGATGCACG |
| | | AGGCCCTGCACAACCACTACACCCAGAAGAGCCTGAGCCTGT |
| | | CCCTGGGCAAGATG |
| SEQ ID | IgD hinge | RWPESPKAQASSVPTAQPQAEGSLAKATTAPATTRNTGRGGEEK |
| NO: 271 | (aa) | KKEKEKEEQEERETKTPECPSHTQPLGVYLLTPAVQDLWLRDKA |
| | | TFTCFVVGSDLKDAHLTWEVAGKVPTGGVEEGLLERHSNGSQSQ |
| | | HSRLTLPRSLWNAGTSVTCTLNHPSLPPQRLMALREPAAQAPVK |
| | | LSLNLLASSDPPEAASWLLCEVSGFSPPNILLMWLEDQREVNTSG |
| | | FAPARPPPQPGSTTFWAWSVLRVPAPPSPQPATYTCVVSHEDSRT |
| | | LLNASRSLEVSYVTDH |
| SEQ ID | IgD hinge | AGGTGGCCCGAAAGTCCCAAGGCCCAGGCATCTAGTGTTCCT |
| NO: 272 | (na) | ACTGCACAGCCCCAGGCAGAAGGCAGCCTAGCCAAAGCTACT |
| | | ACTGCACCTGCCACTACGCGCAATACTGGCCGTGGCGGGGAG |
| | | GAGAAGAAAAAGGAGAAAGAGAAAGAAGAACAGGAAGAGA |
| | | GGGAGACCAAGACCCCTGAATGTCCATCCCATACCCAGCCGC |
| | | TGGGCGTCTATCTCTTGACTCCCGCAGTACAGGACTTGTGGCT |
| | | TAGAGATAAGGCCACCTTTACATGTTTCGTCGTGGGCTCTGAC |
| | | CTGAAGGATGCCCATTTGACTTGGGAGGTTGCCGGAAAGGTA |
| | | CCCACAGGGGGGGTTGAGGAAGGGTTGCTGGAGCGCCATTCC |
| | | AATGGCTCTCAGAGCCAGCACTCAAGACTCACCCTTCCGAGAT |
| | | CCCTGTGGAACGCCGGGACCTCTGTCACATGTACTCTAAATCA |
| | | TCCTAGCCTGCCCCCACAGCGTCTGATGGCCCTTAGAGAGCCA |
| | | GCCGCCCAGGCACCAGTTAAGCTTAGCCTGAATCTGCTCGCCA |
| | | GTAGTGATCCCCCAGAGGCCGCCAGCTGGCTCTTATGCGAAGT |
| | | GTCCGGCTTTAGCCCGCCCAACATCTTGCTCATGTGGCTGGAG |
| | | GACCAGCGAGAAGTGAACACCAGCGGCTTCGCTCCAGCCCGG |
| | | CCCCCACCCCAGCCGGGTTCTACCACATTCTGGGCCTGGAGTG |
| | | TCTTAAGGGTCCCAGCACCACCTAGCCCCCAGCCAGCCACATA |
| | | CACCTGTGTTGTGTCCCATGAAGATAGCAGGACCCTGCTAAAT |
| | | GCTTCTAGGAGTCTGGAGGTTTCCTACGTGACTGACCATT |
| SEQ ID | GS | GGGGSGGGGS |
| NO: 273 | hinge/linker | |
| | (aa) | |
| SEQ ID | GS | GGTGGCGGAGGTTCTGGAGGTGGAGGTTCC |
| NO: 274 | hinge/linker | |
| | (na) | |
| SEQ ID | CD8 | IYIWAPLAGTCGVLLLSLVITLYC |
| NO: 266 | transmembr | |
| | ane (aa) | |
| SEQ ID | CD8 | ATCTACATCTGGGCGCCCTTGGCCGGGACTTGTGGGGTCCTTC |
| NO: 267 | transmembr | TCCTGTCACTGGTTATCACCCTTTACTGC |
| | ane (na) | |
| SEQ ID | CD8 | ATCTACATTTGGGCCCCTCTGGCTGGTACTTGCGGGGTCCTGC |
| NO: 301 | transmembr | TGCTTTCACTCGTGATCACTCTTTACTGT |
| | ane (na) | |
| SEQ ID | 4-1BB | KRGRKKLLYIFKQPFMRPVQTTQEEDGCSCRFPEEEEGGCEL |
| NO: 279 | intracellular | |
| | domain (aa) | |
| SEQ ID | 4-1BB | AAACGGGGCAGAAAGAAACTCCTGTATATATTCAAACAACCA |
| NO: 281 | intracellular | TTTATGAGACCAGTACAAACTACTCAAGAGGAAGATGGCTGT |
| | domain (na) | AGCTGCCGATTTCCAGAAGAAGAAGAAGGAGGATGTGAACTG |
| SEQ ID | 4-1BB | AAGCGCGGTCGGAAGAAGCTGCTGTACATCTTTAAGCAACCC |
| NO: 302 | intracellular | TTCATGAGGCCTGTGCAGACTACTCAAGAGGAGGACGGCTGT |
| | domain (na) | TCATGCCGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPE |
| NO: 280 | | PACSP |
| SEQ ID | CD27 (na) | CAACGAAGGAAATATAGATCAAACAAAGGAGAAAGTCCTGTG |
| NO: 282 | | GAGCCTGCAGAGCCTTGTCGTTACAGCTGCCCCAGGGAGGAG |
| | | GAGGGCAGCACCATCCCCATCCAGGAGGATTACCGAAAACCG |
| | | GAGCCTGCCTGCTCCCCC |
| SEQ ID | CD 3-zeta | RVKFSRSADAPAYKQGQNQLYNELNLGRREEYDVLDKRRGRDP |
| NO: 275 | (aa) | EMGGKPRRKNPQEGLYNELQKDKMAEAYSEIGMKGERRRGKG |
| | | HDGLYQGLSTATKDTYDALHMQALPPR |
| SEQ ID | CD 3-zeta | AGAGTGAAGTTCAGCAGGAGCGCAGACGCCCCCGCGTACAAG |
| NO: 277 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | CD 3-zeta | CGCGTGAAATTCAGCCGCAGCGCAGATGCTCCAGCCTACAAG |
| NO: 303 | (na) | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAAG |
| | | CCTATAGCGAGATTGGTATGAAAGGGGAACGCAGAAGAGGCA |
| | | AAGGCCACGACGGACTGTACCAGGGACTCAGCACCGCCACCA |
| | | AGGACACCTATGACGCTCTTCACATGCAGGCCCTGCCGCCTCG |
| | | G |
| SEQ ID | CD 3-zeta | RVKFSRSADAPAYQQGQNQLYNELNLGRREEYDVLDKRRGRDP |
| NO: 276 | (aa) | EMGGKPRRKNPQEGLYNELQKDKMAEAYSEIGMKGERRRGKG |
| | | HDGLYQGLSTATKDTYDALHMQALPPR |
| SEQ ID | CD 3-zeta | AGAGTGAAGTTCAGCAGGAGCGCAGACGCCCCCGCGTACCAG |
| NO: 278 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | Linker (aa) | GGGGS |
| NO: 304 | | |
| SEQ ID | PD-1 | PGWFLDSPDRPWNPPTFSPALLVVTEGDNATFTCSFSNTSESFVL |
| NO: 305 | extracellular | NWYRMSPSNQTDKLAAFPEDRSQPGQDCRFRVTQLPNGRDFHM |
| | domain (aa) | SVVRARRNDSGTYLCGAISLAPKAQIKESLRAELRVTERRAEVPT |
| | | AHPSPSPRPAGQFQTLV |
| SEQ ID | PD-1 | CCCGGATGGTTTCTGGACTCTCCGGATCGCCCGTGGAATCCCC |
| NO: 306 | extracellular | CAACCTTCTCACCGGCACTCTTGGTTGTGACTGAGGGCGATAA |
| | domain (na) | TGCGACCTTCACGTGCTCGTTCTCCAACACCTCCGAATCATTC |
| | | GTGCTGAACTGGTACCGCATGAGCCCGTCAAACCAGACCGAC |
| | | AAGCTCGCCGCGTTTCCGGAAGATCGGTCGCAACCGGGACAG |
| | | GATTGTCGGTTCCGCGTGACTCAACTGCCGAATGGCAGAGACT |
| | | TCCACATGAGCGTGGTCCGCGCTAGGCGAAACGACTCCGGGA |
| | | CCTACCTGTGCGGAGCCATCTCGCTGGCGCCTAAGGCCCAAAT |
| | | CAAAGAGAGCTTGAGGGCCGAACTGAGAGTGACCGAGCGCAG |
| | | AGCTGAGGTGCCAACTGCACATCCATCCCCATCGCCTCGGCCT |
| | | GCGGGGCAGTTTCAGACCCTGGTC |
| SEQ ID | PD-1 CAR | MALPVTALLLPLALLLHAARPPGWFLDSPDRPWNPPTFSPALLVV |
| NO: 307 | (aa) with | TEGDNATFTCSFSNTSESFVLNWYRMSPSNQTDKLAAFPEDRSQP |
| | signal | GQDCRFRVTQLPNGRDFHMSVVRARRNDSGTYLCGAISLAPKAQ |
| | | IKESLRAELRVTERRAEVPTAHPSPSPRPAGQFQTLVTTTPAPRPP |
| | | TPAPTIASQPLSLRPEACRPAAGGAVHTRGLDFACDIYIWAPLAG |
| | | TCGVLLLSLVITLYCKRGRKKLLYIFKQPFMRPVQTTQEEDGCSC |
| | | RFPEEEEGGCELRVKFSRSADAPAYKQGQNQLYNELNLGRREEY |
| | | DVLDKRRGRDPEMGGKPRRKNPQEGLYNELQKDKMAEAYSEIG |
| | | MKGERRRGKGHDGLYQGLSTATKDTYDALHMQALPPR |
| SEQ ID | PD-1 CAR | ATGGCCCTCCCTGTCACTGCCCTGCTTCTCCCCCTCGCACTCCT |
| NO: 308 | (na) | GCTCCACGCCGCTAGACCACCCGGATGGTTTCTGGACTCTCCG |
| | | GATCGCCCGTGGAATCCCCCAACCTTCTCACCGGCACTCTTGG |
| | | TTGTGACTGAGGGCGATAATGCGACCTTCACGTGCTCGTTCTC |
| | | CAACACCTCCGAATCATTCGTGCTGAACTGGTACCGCATGAGC |
| | | CCGTCAAACCAGACCGACAAGCTCGCCGCGTTTCCGGAAGAT |
| | | CGGTCGCAACCGGGACAGGATTGTCGGTTCCGCGTGACTCAA |
| | | CTGCCGAATGGCAGAGACTTCCACATGAGCGTGGTCCGCGCT |
| | | AGGCGAAACGACTCCGGGACCTACCTGTGCGGAGCCATCTCG |
| | | CTGGCGCCTAAGGCCCAAATCAAAGAGAGCTTGAGGGCCGAA |
| | | CTGAGAGTGACCGAGCGCAGAGCTGAGGTGCCAACTGCACAT |
| | | CCATCCCCATCGCCTCGGCCTGCGGGGCAGTTTCAGACCCTGG |
| | | TCACGACCACTCCGGCGCCGCGCCCACCGACTCCGGCCCCAAC |
| | | TATCGCGAGCCAGCCCCTGTCGCTGAGGCCGGAAGCATGCCG |
| | | CCCTGCCGCCGGAGGTGCTGTGCATACCCGGGGATTGGACTTC |
| | | GCATGCGACATCTACATTTGGGCTCCTCTCGCCGGAACTTGTG |
| | | GCGTGCTCCTTCTGTCCCTGGTCATCACCCTGTACTGCAAGCG |
| | | GGGTCGGAAAAAGCTTCTGTACATTTTCAAGCAGCCCTTCATG |
| | | AGGCCCGTGCAAACCACCCAGGAGGAGGACGGTTGCTCCTGC |
| | | CGGTTCCCCGAAGAGGAAGAAGGAGGTTGCGAGCTGCGCGTG |
| | | AAGTTCTCCCGGAGCGCCGACGCCCCCGCCTATAAGCAGGGC |
| | | CAGAACCAGCTGTACAACGAACTGAACCTGGGACGGCGGGAA |
| | | GAGTACGATGTGCTGGACAAGCGGCGCGGCCGGGACCCCGAA |
| | | ATGGGCGGGAAGCCTAGAAGAAAGAACCCTCAGGAAGGCCTG |
| | | TATAACGAGCTGCAGAAGGACAAGATGGCCGAGGCCTACTCC |
| | | GAAATTGGGATGAAGGGAGAGCGGCGGAGGGGAAAGGGGCA |
| | | CGACGGCCTGTACCAAGGACTGTCCACCGCCACCAAGGACAC |
| | | ATACGATGCCCTGCACATGCAGGCCCTTCCCCCTCGC |
| SEQ ID | Linker (aa) | (Gly-Gly-Gly-Ser)n, where n = 1-10 |
| NO: 232 | | |
| SEQ ID | Linker (aa) | (Gly4 Ser)4 |
| NO: 230 | | |
| SEQ ID | Linker (aa) | (Gly4 Ser)3 |
| NO: 231 | | |
| SEQ ID | Linker (aa) | (Gly3Ser) |
| NO: 309 | | |
| SEQ ID | polyA (na) | [a]₅₀₋₅₀₀₀ |
| NO: 310 | | |
| SEQ ID | PD1 CAR | PGWFLDSPDRPWNPPTFSPALLVVTEGDNATFTCSFSNTSESF |
| NO: 311 | (aa) | VLNWYRMSPSNQTDKLAAFPEDRSQPGQDCRFRVTQLPNGRDFHM |
| | | SVVRARRNDSGTYLCGAISLAPKAQIKESLRAELRVTERRAEVPT |
| | | AHPSPSPRPAGQFQTLVTTTPAPRPPTPAPTIASQPLSLRPEACR |
| | | PAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRS |
| | | ADAPAYKQGQNQLYNELNLGRREEYDVLDKRRGRDPEMGGKPR |
| | | RKNPQEGLYNELQKDKMAEAYSEIGMKGERRRGKGHDGLYQG |
| | | LSTATKDTYDALHMQALPPR |
| SEQ ID | ICOS | TKKKYSSSVHDPNGEYMFMRAVNTAKKSRLTDVTL |
| NO: 312 | intracellular | |
| | domain (aa) | |
| SEQ ID | ICOS | ACAAAAAAGAAGTATTCATCCAGTGTGCACGACCCTAACGGT |
| NO: 313 | intracellular | GAATACATGTTCATGAGAGCAGTGAACACAGCCAAAAAATCC |
| | domain (na) | AGACTCACAGATGTGACCCTA |
| SEQ ID | ICOS TM | TTTPAPRPPTPAPTIASQPLSLRPEACRPAAGGAVHTRGLDFA |
| NO: 314 | domain (aa) | CDFWLPIGCAAFVVVCILGCILICWL |
| SEQ ID | ICOS TM | ACCACGACGCCAGCGCCGCGACCACCAACACCGGCGCCCACC |
| NO: 315 | domain (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGATTTCTGGTTACCCATAGGATGTGCAGCCTTTGTTGTA |
| | | GTCTGCATTTTGGGATGCATACTTATTTGTTGGCTT |
| SEQ ID | CD28 | RSKRSRLLHSDYMNMTPRRPGPTRKHYQPYAPPRDFAAYRS |
| NO: 316 | intracellular | |
| | domain (aa) | |
| SEQ ID | CD28 | AGGAGTAAGAGGAGCAGGCTCCTGCACAGTGACTACATGAAC |
| NO: 317 | intracellular | ATGACTCCCCGCCGCCCCGGGCCCACCCGCAAGCATTACCAGC |
| | domain (na) | CCTATGCCCCACCACGCGACTTCGCAGCCTATCGCTCC |

### Targets

The present disclosure provides cells, e.g., immune effector cells (e.g., T cells, NK cells), that comprise or at any time comprised a gRNA molecule or CRISPR system as described herein, that are further engineered to contain one or more CARs that direct the immune effector cells to undesired cells (e.g., cancer cells). This is achieved through an antigen binding domain on the CAR that is specific for a cancer associated antigen. There are two classes of cancer associated antigens (tumor antigens) that can be targeted by the CARs of the instant disclosure: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

In some embodiments, the tumor antigen is selected from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma. Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is selected from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is selected from one or more of: CD33, CD11b, C14, CD15, and CD66b. Accordingly, in some embodiments, the tumor-supporting antigen is selected from one or more of: bone marrow stromal cell antigen 2 (BST2), fibroblast activation protein (FAP) or tenascin, CD33, CD11b, C14, CD15, and CD66b.

### Antigen Binding Domain Structures

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 232). In one embodiment, the linker can be (Gly₄Ser)₄ (SEQ ID NO: 230) or (Gly₄Ser)₃ (SEQ ID NO: 231). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In one embodiment, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the antigen binding domain has a binding affinity that is at least five-fold, 10-fold, 20-fold, 30-fold, 50-fold, 100-fold or 1,000-fold less than a reference antibody, e.g., an antibody described herein. In one embodiment, the encoded antigen binding domain has a binding affinity at least 5-fold less than a reference antibody (e.g., an antibody from which the antigen binding domain is derived). In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan.

In one aspect, the antigen binding domain of the CAR is a scFv antibody fragment that is humanized compared to the murine sequence of the scFv from which it is derived.

In one aspect, the antigen binding domain of a CAR of the disclosure (e.g., a scFv) is encoded by a nucleic acid molecule whose sequence has been codon optimized for expression in a mammalian cell. In one aspect, entire CAR construct of the disclosure is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

### Antigen Binding Domains (and the Targeted Antigens)

In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In one embodiment, an antigen binding domain against mesothelin is an antigen binding portion, e.g., CDRs, of an antibody, antigen-binding fragment or CAR described in, e.g., PCT publication WO2015/090230. In one embodiment, an antigen binding domain against mesothelin is an antigen binding portion, e.g., CDRs, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419. In one embodiment, an antigen binding domain against mesothelin is an antigen binding portion, e.g., CDRs, of an antibody, antigen-binding fragment, or CAR described in WO/2015/090230.

In one embodiment, an antigen binding domain against CD123 is an antigen binding portion, e.g., CDRs, of an antibody, antigen-binding fragment or CAR described in, e.g., PCT publication WO2014/130635. In one embodiment, an antigen binding domain against CD123 is an antigen binding portion, e.g., CDRs, of an antibody, antigen-binding fragment, or CAR described in, e.g., PCT publication WO2014/138805, WO2014/138819, WO2013/173820, WO2014/144622, WO2001/66139, WO2010/126066, WO2014/144622, or US2009/0252742. In one embodiment, an antigen binding domain against CD123 is an antigen binding portion, e.g., CDRs, of an antibody, antigen-binding fragment, or CAR described in WO/2016/028896.

In one embodiment, an antigen binding domain against EGFRvIII is an antigen binding portion, e.g., CDRs, of an antibody, antigen-binding fragment or CAR described in, e.g., WO/2014/130657.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD). In one embodiment, an antigen binding domain against CLL-1 is an antigen binding portion, e.g., CDRs, of an antibody, antigen-binding fragment, or CAR described in WO/2016/014535.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). In one embodiment, an antigen binding domain against CD33 is an antigen binding portion, e.g., CDRs, of an antibody, antigen-binding fragment, or CAR described in WO/2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In one embodiment, an antigen binding domain against BCMA is an antigen binding portion, e.g., CDRs, of an antibody, antigen-binding fragment, or CAR described in WO/2016/014565.

In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRS S21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131(2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Malian et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against S SEA-4 is an antigen binding portion, e.g., CDRs, of antibody MC813 (Cell Signalling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., U520100297138; or WO2007/067992.

In one embodiment, an antigen binding domain against sLe is an antigen binding portion, e.g., CDRs, of the antibody G193 (for Lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRCSD is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J. 15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signalling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med.

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748—Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9, 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1xCD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32. In one embodiment, an antigen binding domain against FCRL5 is an antigen binding portion, e.g., CDRs, of the anti-FcRL5 antibody described in, for example, WO2001/038490, WO/2005/117986, WO2006/039238, WO2006/076691, WO2010/114940, WO2010/120561, or WO2014/210064.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

In an embodiment, the antigen-binding domain of a CAR, e.g., a CAR expressed by a cell of the disclosure, binds to CD19. CD19 is found on B cells throughout differentiation of the lineage from the pro/pre-B cell stage through the terminally differentiated plasma cell stage. In an embodiment, the antigen binding domain is a murine scFv domain that binds to human CD19, e.g., the antigen binding domain of CTL019 (e.g., SEQ ID NO: 252). In an embodiment, the antigen binding domain is a humanized antibody or antibody fragment, e.g., scFv domain, derived from the murine CTL019 scFv. In an embodiment, the antigen binding domain is a human antibody or antibody fragment that binds to human CD19. Exemplary scFv domains (and their sequences, e.g., CDRs, VL and VH sequences) that bind to CD19 are provided in Table 12a. The scFv domain sequences provided in Table 12a include a light chain variable region (VL) and a heavy chain variable region (VH). The VL and VH are attached by a linker comprising the sequence GGGGSGGGGSGGGGS (SEQ ID NO: 231), e.g., in the following orientation: VL-linker-VH.

TABLE 12a
Antigen Binding domains that bind CD 19

| | | | SEQ |
|---|---|---|---|
| | | | ID |
| Antigen | Name | Amino Acid Sequence | NO: |
| CD19 | muCTL | DIQMTQTTSSLSASLGDRVTISCRASQDISKYLNWYQQKPDGTV | 233 |
| | 019 | KLLIYHTSRLHSGVPSRFSGSGSGTDYSLTISNLEQEDIATYFCQ | |
| | | QGNTLPYTFGGGTKLEITGGGGSGGGGSGGGGSEVKLQESGPGL | |
| | | VAPSQSLSVTCTVSGVSLPDYGVSWIRQPPRKGLEWLGVIWGSE | |
| | | TTYYNSALKSRLTIIKDNSKSQVFLKMNSLQTDDTAIYYCAKHY | |
| | | YYGGSYAMDYWGQGTSVTVSS | |
| CD19 | huscFv1 | EIVMTQSPATLSLSPGERATLSCRASQDISKYLNWYQQKPGQAP | 234 |
| | | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYSSSLKSRVTISKDNSKNQVSLKLSSVTAADTAVYYCAKH | |
| | | YYYGGSYAMDYWGQGTLVTVSS | |
| CD19 | huscFv2 | EIVMTQSPATLSLSPGERATLSCRASQDISKYLNWYQQKPGQAP | 235 |
| | | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYQSSLKSRVTISKDNSKNQVSLKLSSVTAADTAVYYCAKH | |
| | | YYYGGSYAMDYWGQGTLVTVSS | |
| CD19 | huscFv3 | QVQLQESGPGLVKPSETLSLTCTVSGVSLPDYGVSWIRQPPGKG | 236 |
| | | LEWIGVIWGSETTYYSSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |
| CD19 | huscFv4 | QVQLQESGPGLVKPSETLSLTCTVSGVSLPDYGVSWIRQPPGKG | 237 |
| | | LEWIGVIWGSETTYYQSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |
| CD19 | huscFv5 | EIVMTQSPATLSLSPGERATLSCRASQDISKYLNWYQQKPGQAP | 238 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYSSSLKSRVTISKDNSKNQVSLKLSSVTAADTAVYYC | |
| | | AKHYYYGGSYAMDYWGQGTLVTVSS | |
| CD19 | huscFv6 | EIVMTQSPATLSLSPGERATLSCRASQDISKYLNWYQQKPGQAP | 239 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYQSSLKSRVTISKDNSKNQVSLKLSSVTAADTAVYY | |
| | | CAKHYYYGGSYAMDYWGQGTLVTVSS | |
| CD19 | huscFv7 | QVQLQESGPGLVKPSETLSLTCTVSGVSLPDYGVSWIRQPPGKG | 240 |
| | | LEWIGVIWGSETTYYSSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | huscFv8 | QVQLQESGPGLVKPSETLSLTCTVSGVSLPDYGVSWIRQPPGKG | 241 |
| | | LEWIGVIWGSETTYYQSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | huscFv9 | EIVMTQSPATLSLSPGERATLSCRASQDISKYLNWYQQKPGQAP | 242 |
| | | RLLIYHTSRLHSGIPARFSGSGSGTDYTLTISSLQPEDFAVYFCQQ | |
| | | GNTLPYTFGQGTKLEIKGGGGSGGGGSGGGGSGGGGSQVQLQE | |
| | | SGPGLVKPSETLSLTCTVSGVSLPDYGVSWIRQPPGKGLEWIGVI | |
| | | WGSETTYYNSSLKSRVTISKDNSKNQVSLKLSSVTAADTAVYY | |
| | | CAKHYYYGGSYAMDYWGQGTLVTVSS | |
| CD19 | Hu | QVQLQESGPGLVKPSETLSLTCTVSGVSLPDYGVSWIRQPPGKG | 243 |
| | scFv10 | LEWIGVIWGSETTYYNSSLKSRVTISKDNSKNQVSLKLSSVTAA | |
| | | DTAVYYCAKHYYYGGSYAMDYWGQGTLVTVSSGGGGSGGGG | |
| | | SGGGGSGGGGSEIVMTQSPATLSLSPGERATLSCRASQDISKYL | |
| | | NWYQQKPGQAPRLLIYHTSRLHSGIPARFSGSGSGTDYTLTISSL | |
| | | QPEDFAVYFCQQGNTLPYTFGQGTKLEIK | |
| CD19 | Hu | EIVMTQSPATLSLSPGERATLSCRASQDISKYLNWYQQKPGQAP | 244 |
| | scFv11 | RLLIYHTSRLHSGIPARFSGSGSGTDYTLTISSLQPEDFAVYFCQQ | |
| | | GNTLPYTFGQGTKLEIKGGGGSGGGGSGGGGSQVQLQESGPGL | |
| | | VKPSETLSLTCTVSGVSLPDYGVSWIRQPPGKGLEWIGVIWGSE | |
| | | TTYYNSSLKSRVTISKDNSKNQVSLKLSSVTAADTAVYYCAKH | |
| | | YYYGGSYAMDYWGQGTLVTVSS | |
| CD19 | Hu | QVQLQESGPGLVKPSETLSLTCTVSGVSLPDYGVSWIRQPPGKG | 245 |
| | scFv12 | LEWIGVIWGSETTYYNSSLKSRVTISKDNSKNQVSLKLSSVTAA | |
| | | DTAVYYCAKHYYYGGSYAMDYWGQGTLVTVSSGGGGSGGGG | |
| | | SGGGGSEIVMTQSPATLSLSPGERATLSCRASQDISKYLNWYQQ | |
| | | KPGQAPRLLIYHTSRLHSGIPARFSGSGSGTDYTLTISSLQPEDFA | |
| | | VYFCQQGNTLPYTFGQGTKLEIK | |

The sequences of the CDR sequences of the scFv domains of the CD19 antigen binding domains provided in Table 12a are shown in Table 12b for the heavy chain variable domains and in Table 12c for the light chain variable domains “ID” stands for the respective SEQ ID NO for each CDR.

TABLE 12b
Heavy Chain Variable Domain CDRs

| Description FW | HCDR1 | ID | HCDR2 | ID | HCDR3 | ID |
|---|---|---|---|---|---|---|
| murine_ | GVSLP | 319 | VIWGSE | 320 | HYYYG | 246 |
| CART19 | DYGVS | | TTYYNS | | GSYAM | |
| | | | ALKS | | DY | |
| humanized_ VH4 | GVSLP | 319 | VIWGSE | 296 | HYYYG | 246 |
| CART19 | DYGVS | | TTYY [symbol] S | | GSYAM | |
| a | | | [symbol] LKS | | DY | |
| humanized_ VH4 | GVSLP | 319 | VIWGSE | 295 | HYYYG | 246 |
| CART19 | DYGVS | | TTYY [symbol] S | | GSYAM | |
| b | | | SLKS | | DY | |
| humanized_ VH4 | GVSLP | 319 | VIWGSE | 284 | HYYYG | 246 |
| CART19 | DYGVS | | TTYYNS | | GSYAM | |
| c | | | SLKS | | DY | |

TABLE 12c
Light Chain Variable Domain CDRs

| Description | FW | LCDR1 | ID | LCDR2 | ID | LCDR3 | ID |
|---|---|---|---|---|---|---|---|
| murine_ | | RASQDI | 251 | HTSRLHS | 250 | QQGNTLPYT | 247 |
| CART19 | | SKYLN | | | | | |
| humanized_ | VK3 | RASQDI | 251 | HTSRLHS | 250 | QQGNTLPYT | 247 |
| CART19 a | | SKYLN | | | | | |
| humanized_ | VK3 | RASQDI | 251 | HTSRLHS | 250 | QQGNTLPYT | 247 |
| CART19 b | | SKYLN | | | | | |
| humanized_ | VK3 | RASQDI | 251 | HTSRLHS | 250 | QQGNTLPYT | 247 |
| CART19 c | | SKYLN | | | | | |

In an embodiment, the antigen binding domain comprises an anti-CD19 antibody, or fragment thereof, e.g., a scFv. For example, the antigen binding domain comprises a variable heavy chain and a variable light chain listed in Table 12d. The linker sequence joining the variable heavy and variable light chains can be any of the linker sequences described herein, or alternatively, can be GSTSGSGKPGSGEGSTKG (SEQ ID NO: 248). The light chain variable region and heavy chain variable region of a scFv can be, e.g., in any of the following orientations: light chain variable region-linker-heavy chain variable region or heavy chain variable region-linker-light chain variable region.

TABLE 12d
Additional Anti-CD19 antibody binding domains

| Ab | | |
|---|---|---|
| Name | VH Sequence | VL Sequence |
| SJ25-C1 | QVQLLESGAELVRP | ELVLTQSPKFMST |
| | GSSVKISCKASGYA | SVGDRVSVTCKAS |
| | FSSYWMNWVKQRPG | QNVGTNVAWYQQK |
| | QGLEWIGQIYPGD | PGQSPKPLIYSAT |
| | GDTNYNGKFKGQAT | YRNSGVPDRFTGS |
| | LTADKSSSTAYMQ | GSGTDFTLTITNV |
| | LSGLTSEDSAVYSC | QSKDLADYFYFCQ |
| | ARKTISSVVDFYFD | YNRYPYTSGGGT |
| | YWGQGTTVT | KLEIKRRS |
| | (SEQ ID NO: 249) | (SEQ ID NO: 229) |

| | |
|---|---|
| | ScFv Sequence |
| SJ25-C1 | QVQLLESGAELVRPGSSVKISCKASGYA |
| scFv | FSSYWMNWVKQRPGQGLEWIGQIYPGD |
| | GDTNYNGKFKGQATLTADKSSSTAYMQL |
| | SGLTSEDSAVYSCARKTISSVVDFYFDY |
| | WGQGTTVTGSTSGSGKPGSGEGSTKGEL |
| | VLTQSPKFMSTSVGDRVSVTCKASQNVG |
| | TNVAWYQQKPGQSPKPLIYSATYRNSGV |
| | PDRFTGSGSGTDFTLTITNVQSKDLADY |
| | FYFCQYNRYPYTSGGGTKLEIKRRS |
| | (SEQ ID NO: 228) |

In one embodiment, the CD19 binding domain comprises one or more (e.g., all three) light chain complementary determining region 1 (LC CDR1), light chain complementary determining region 2 (LC CDR2), and light chain complementary determining region 3 (LC CDR3) of a CD19 binding domain described herein, e.g., provided in Table 12a or 15, and/or one or more (e.g., all three) heavy chain complementary determining region 1 (HC CDR1), heavy chain complementary determining region 2 (HC CDR2), and heavy chain complementary determining region 3 (HC CDR3) of a CD19 binding domain described herein, e.g., provided in Table 12a or 16. In one embodiment, the CD19 binding domain comprises one, two, or all of LC CDR1, LC CDR2, and LC CDR3 of any amino acid sequences as provided in Table 12c; and one, two or all of HC CDR1, HC CDR2, and HC CDR3 of any amino acid sequences as provided in Table 12b.

Any known CD19 CAR, e.g., the CD19 antigen binding domain of any known CD19 CAR, in the art can be used in accordance with the instant disclosure to construct a CAR. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10. In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In an embodiment, the antigen-binding domain of CAR, e.g., a CAR expressed by a cell of the disclosure, binds to BCMA. BCMA is found preferentially expressed in mature B lymphocytes. In an embodiment, the antigen binding domain is a murine scFv domain that binds to human BCMA. In an embodiment, the antigen binding domain is a humanized antibody or antibody fragment, e.g., scFv domain that binds human B CMA. In an embodiment, the antigen binding domain is a human antibody or antibody fragment that binds to human BCMA. In embodiments, exemplary BCMA CAR constructs are generated using the VH and VL sequences from PCT Publication WO2012/0163805. In embodiments, additional exemplary BCMA CAR constructs are generated using the VH and VL sequences from PCT Publication WO2016/014565. In embodiments, additional exemplary BCMA CAR constructs are generated using the VH and VL sequences from PCT Publication WO2014/122144. In embodiments, additional exemplary BCMA CAR constructs are generated using the CAR molecules, and/or the VH and VL sequences from PCT Publication WO2016/014789. In embodiments, additional exemplary BCMA CAR constructs are generated using the CAR molecules, and/or the VH and VL sequences from PCT Publication WO2014/089335. In embodiments, additional exemplary BCMA CAR constructs are generated using the CAR molecules, and/or the VH and VL sequences from PCT Publication WO2014/140248.

Any known BCMA CAR, e.g., the BMCA antigen binding domain of any known BCMA CAR, in the art can be used in accordance with the instant disclosure. For example, those described herein.

### Exemplary CAR Molecules

In one aspect, a CAR, e.g., a CAR expressed by the cell of the disclosure, comprises a CAR molecule comprising an antigen binding domain that binds to a B cell antigen, e.g., as described herein, such as CD19 or BCMA.

In one embodiment, the CAR comprises a CAR molecule comprising a CD19 antigen binding domain (e.g., a murine, human or humanized antibody or antibody fragment that specifically binds to CD19), a transmembrane domain, and an intracellular signalling domain (e.g., an intracellular signalling domain comprising a costimulatory domain and/or a primary signalling domain).

Exemplary CAR molecules described herein are provided in Table 12e. The CAR molecules in Table 12e comprise a CD19 antigen binding domain, e.g., an amino acid sequence of any CD19 antigen binding domain provided in Table 12a.

TABLE 12e
Exemplary CD19 CAR molecules

| | | | SEQ |
|---|---|---|---|
| | | | ID |
| Antigen | Name | Amino Acid Sequence | NO: |
| CD 19 | CTL019 | MALPVTALLLPLALLLHAARPDIQMTQTTSSLSASLGDRVTISCR | 252 |
| | | ASQDISKYLNWYQQKPDGTVKLLIYHTSRLHSGVPSRFSGSGSG | |
| | | TDYSLTISNLEQEDIATYFCQQGNTLPYTFGGGTKLEITGGGGSG | |
| | | GGGSGGGGSEVKLQESGPGLVAPSQSLSVTCTVSGVSLPDYGVS | |
| | | WIRQPPRKGLEWLGVIWGSETTYYNSALKSRLTIIKDNSKSQVFL | |
| | | KMNSLQTDDTAIYYCAKHYYYGGSYAMDYWGQGTSVTVSSTT | |
| | | TPAPRPPTPAPTIASQPLSLRPEACRPAAGGAVHTRGLDFACDIYI | |
| | | WAPLAGTCGVLLLSLVITLYCKRGRKKLLYIFKQPFMRPVQTTQ | |
| | | EEDGCSCRFPEEEEGGCELRVKFSRSADAPAYKQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CD19 | CAR 1 | MALPVTALLLPLALLLHAARPEIVMTQSPATLSLSPGERATLSCR | 253 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYSSSLKSRVTISKDNSKNQVSLK | |
| | | LSSVTAADTAVYYCAKHYYYGGSYAMDYWGQGTLVTVSSTTTP | |
| | | APRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR 2 | MALPVTALLLPLALLLHAARPEIVMTQSPATLSLSPGERATLSCR | 254 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYQSSLKSRVTISKDNSKNQVSLK | |
| | | LSSVTAADTAVYYCAKHYYYGGSYAMDYWGQGTLVTVSSTTT | |
| | | PAPRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |
| CD19 | CAR 3 | MALPVTALLLPLALLLHAARPQVQLQESGPGLVKPSETLSLTCT | 255 |
| | | VSGVSLPDYGVSWIRQPPGKGLEWIGVIWGSETTYYSSSLKSRVT | |
| | | ISKDNSKNQVSLKLSSVTAADTAVYYCAKHYYYGGSYAMDYW | |
| | | GQGTLVTVSSGGGGSGGGGSGGGGSEIVMTQSPATLSLSPGERA | |
| | | TLSCRASQDISKYLNWYQQKPGQAPRLLIYHTSRLHSGIPARFSG | |
| | | SGSGTDYTLTISSLQPEDFAVYFCQQGNTLPYTFGQGTKLEIKTT | |
| | | TPAPRPPTPAPTIASQPLSLRPEACRPAAGGAVHTRGLDFACDIYI | |
| | | WAPLAGTCGVLLLSLVITLYCKRGRKKLLYIFKQPFMRPVQTTQ | |
| | | EEDGCSCRFPEEEEGGCELRVKFSRSADAPAYKQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CD19 | CAR 4 | MALPVTALLLPLALLLHAARPQVQLQESGPGLVKPSETLSLTCT | 256 |
| | | VSGVSLPDYGVSWIRQPPGKGLEWIGVIWGSETTYYQSSLKSRV | |
| | | TISKDNSKNQVSLKLSSVTAADTAVYYCAKHYYYGGSYAMDY | |
| | | WGQGTLVTVSSGGGGSGGGGSGGGGSEIVMTQSPATLSLSPGER | |
| | | ATLSCRASQDISKYLNWYQQKPGQAPRLLIYHTSRLHSGIPARFS | |
| | | GSGSGTDYTLTISSLQPEDFAVYFCQQGNTLPYTFGQGTKLEIKT | |
| | | TTPAPRPPTPAPTIASQPLSLRPEACRPAAGGAVHTRGLDFACDIY | |
| | | IWAPLAGTCGVLLLSLVITLYCKRGRKKLLYIFKQPFMRPVQTTQ | |
| | | EEDGCSCRFPEEEEGGCELRVKFSRSADAPAYKQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CD19 | CAR 5 | MALPVTALLLPLALLLHAARPEIVMTQSPATLSLSPGERATLSCR | 257 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYSSSLKSRVTISKDNSKN | |
| | | QVSLKLSSVTAADTAVYYCAKHYYYGGSYAMDYWGQGTLVT | |
| | | VSSTTTPAPRPPTPAPTIASQPLSLRPEACRPAAGGAVHTRGLDFA | |
| | | CDIYIWAPLAGTCGVLLLSLVITLYCKRGRKKLLYIFKQPFMRPV | |
| | | QTTQEEDGCSCRFPEEEEGGCELRVKFSRSADAPAYKQGQNQLY | |
| | | NELNLGRREEYDVLDKRRGRDPEMGGKPRRKNPQEGLYNELQK | |
| | | DKMAEAYSEIGMKGERRRGKGHDGLYQGLSTATKDTYDALHM | |
| | | QALPPR | |
| CD19 | CAR 6 | MALPVTALLLPLALLLHAARPEIVMTQSPATLSLSPGERATLSCR | 258 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYQSSLKSRVTISKDNSK | |
| | | NQVSLKLSSVTAADTAVYYCAKHYYYGGSYAMDYWGQGTLV | |
| | | TVSSTTTPAPRPPTPAPTIASQPLSLRPEACRPAAGGAVHTRGLDF | |
| | | ACDIYIWAPLAGTCGVLLLSLVITLYCKRGRKKLLYIFKQPFMRP | |
| | | VQTTQEEDGCSCRFPEEEEGGCELRVKFSRSADAPAYKQGQNQL | |
| | | YNELNLGRREEYDVLDKRRGRDPEMGGKPRRKNPQEGLYNELQ | |
| | | KDKMAEAYSEIGMKGERRRGKGHDGLYQGLSTATKDTYDALH | |
| | | MQALPPR | |
| CD19 | CAR 7 | MALPVTALLLPLALLLHAARPQVQLQESGPGLVKPSETLSLTCT | 259 |
| | | VSGVSLPDYGVSWIRQPPGKGLEWIGVIWGSETTYYSSSLKSRVT | |
| | | ISKDNSKNQVSLKLSSVTAADTAVYYCAKHYYYGGSYAMDYW | |
| | | GQGTLVTVSSGGGGSGGGGSGGGGSGGGGSEIVMTQSPATLSLS | |
| | | PGERATLSCRASQDISKYLNWYQQKPGQAPRLLIYHTSRLHSGIP | |
| | | ARFSGSGSGTDYTLTISSLQPEDFAVYFCQQGNTLPYTFGQGTKL | |
| | | EIKTTTPAPRPPTPAPTIASQPLSLRPEACRPAAGGAVHTRGLDFA | |
| | | CDIYIWAPLAGTCGVLLLSLVITLYCKRGRKKLLYIFKQPFMRPV | |
| | | QTTQEEDGCSCRFPEEEEGGCELRVKFSRSADAPAYKQGQNQLY | |
| | | NELNLGRREEYDVLDKRRGRDPEMGGKPRRKNPQEGLYNELQK | |
| | | DKMAEAYSEIGMKGERRRGKGHDGLYQGLSTATKDTYDALHM | |
| | | QALPPR | |
| CD19 | CAR 8 | MALPVTALLLPLALLLHAARPQVQLQESGPGLVKPSETLSLTCT | 260 |
| | | VSGVSLPDYGVSWIRQPPGKGLEWIGVIWGSETTYYQSSLKSRV | |
| | | TISKDNSKNQVSLKLSSVTAADTAVYYCAKHYYYGGSYAMDY | |
| | | WGQGTLVTVSSGGGGSGGGGSGGGGSGGGGSEIVMTQSPATLS | |
| | | LSPGERATLSCRASQDISKYLNWYQQKPGQAPRLLIYHTSRLHSG | |
| | | IPARFSGSGSGTDYTLTISSLQPEDFAVYFCQQGNTLPYTFGQGT | |
| | | KLEIKTTTPAPRPPTPAPTIASQPLSLRPEACRPAAGGAVHTRGLD | |
| | | FACDIYIWAPLAGTCGVLLLSLVITLYCKRGRKKLLYIFKQPFMR | |
| | | PVQTTQEEDGCSCRFPEEEEGGCELRVKFSRSADAPAYKQGQNQ | |
| | | LYNELNLGRREEYDVLDKRRGRDPEMGGKPRRKNPQEGLYNEL | |
| | | QKDKMAEAYSEIGMKGERRRGKGHDGLYQGLSTATKDTYDAL | |
| | | HMQALPPR | |
| CD19 | CAR 9 | MALPVTALLLPLALLLHAARPEIVMTQSPATLSLSPGERATLSCR | 261 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYNSSLKSRVTISKDNSK | |
| | | NQVSLKLSSVTAADTAVYYCAKHYYYGGSYAMDYWGQGTLV | |
| | | TVSSTTTPAPRPPTPAPTIASQPLSLRPEACRPAAGGAVHTRGLDF | |
| | | ACDIYIWAPLAGTCGVLLLSLVITLYCKRGRKKLLYIFKQPFMRP | |
| | | VQTTQEEDGCSCRFPEEEEGGCELRVKFSRSADAPAYKQGQNQL | |
| | | YNELNLGRREEYDVLDKRRGRDPEMGGKPRRKNPQEGLYNELQ | |
| | | KDKMAEAYSEIGMKGERRRGKGHDGLYQGLSTATKDTYDALH | |
| | | MQALPPR | |
| CD19 | CAR 10 | MALPVTALLLPLALLLHAARPEIVMTQSPATLSLSPGERATLSCR | 262 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSGGGGSQVQLQESGPGLVKPSETLSLTCTVSGVSLPD | |
| | | YGVSWIRQPPGKGLEWIGVIWGSETTYYNSSLKSRVTISKDNSK | |
| | | NQVSLKLSSVTAADTAVYYCAKHYYYGGSYAMDYWGQGTLV | |
| | | TVSSTTTPAPRPPTPAPTIASQPLSLRPEACRPAAGGAVHTRGLDF | |
| | | ACDIYIWAPLAGTCGVLLLSLVITLYCKRGRKKLLYIFKQPFMRP | |
| | | VQTTQEEDGCSCRFPEEEEGGCELRVKFSRSADAPAYKQGQNQL | |
| | | YNELNLGRREEYDVLDKRRGRDPEMGGKPRRKNPQEGLYNELQ | |
| | | KDKMAEAYSEIGMKGERRRGKGHDGLYQGLSTATKDTYDALH | |
| | | MQALPPR | |
| CD19 | CAR 11 | MALPVTALLLPLALLLHAARPQVQLQESGPGLVKPSETLSLTCT | 263 |
| | | VSGVSLPDYGVSWIRQPPGKGLEWIGVIWGSETTYYNSSLKSRV | |
| | | TISKDNSKNQVSLKLSSVTAADTAVYYCAKHYYYGGSYAMDY | |
| | | WGQGTLVTVSSGGGGSGGGGSGGGGSGGGGSEIVMTQSPATLS | |
| | | LSPGERATLSCRASQDISKYLNWYQQKPGQAPRLLIYHTSRLHSG | |
| | | IPARFSGSGSGTDYTLTISSLQPEDFAVYFCQQGNTLPYTFGQGT | |
| | | KLEIKTTTPAPRPPTPAPTIASQPLSLRPEACRPAAGGAVHTRGLD | |
| | | FACDIYIWAPLAGTCGVLLLSLVITLYCKRGRKKLLYIFKQPFMR | |
| | | PVQTTQEEDGCSCRFPEEEEGGCELRVKFSRSADAPAYKQGQNQ | |
| | | LYNELNLGRREEYDVLDKRRGRDPEMGGKPRRKNPQEGLYNEL | |
| | | QKDKMAEAYSEIGMKGERRRGKGHDGLYQGLSTATKDTYDAL | |
| | | HMQALPPR | |
| CD19 | CAR 12 | MALPVTALLLPLALLLHAARPEIVMTQSPATLSLSPGERATLSCR | 264 |
| | | ASQDISKYLNWYQQKPGQAPRLLIYHTSRLHSGIPARFSGSGSGT | |
| | | DYTLTISSLQPEDFAVYFCQQGNTLPYTFGQGTKLEIKGGGGSGG | |
| | | GGSGGGGSQVQLQESGPGLVKPSETLSLTCTVSGVSLPDYGVSW | |
| | | IRQPPGKGLEWIGVIWGSETTYYNSSLKSRVTISKDNSKNQVSLK | |
| | | LSSVTAADTAVYYCAKHYYYGGSYAMDYWGQGTLVTVSSTTT | |
| | | PAPRPPTPAPTIASQPLSLRPEACRPAAGGAVHTRGLDFACDIYIW | |
| | | APLAGTCGVLLLSLVITLYCKRGRKKLLYIFKQPFMRPVQTTQEE | |
| | | DGCSCRFPEEEEGGCELRVKFSRSADAPAYKQGQNQLYNELNLG | |
| | | RREEYDVLDKRRGRDPEMGGKPRRKNPQEGLYNELQKDKMAE | |
| | | AYSEIGMKGERRRGKGHDGLYQGLSTATKDTYDALHMQALPPR | |

In one aspect, a CAR, e.g., a CAR expressed by the cell of the disclosure, comprises a CAR molecule comprising an antigen binding domain that binds to BCMA, e.g., comprises a BCMA antigen binding domain (e.g., a murine, human or humanized antibody or antibody fragment that specifically binds to BCMA, e.g., human BCMA), a transmembrane domain, and an intracellular signalling domain (e.g., an intracellular signalling domain comprising a costimulatory domain and/or a primary signalling domain).

Exemplary CAR molecules of a CAR described herein are provided in Table 1 of WO2016/014565.

### Transmembrane Domains

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR e.g., in one embodiment, the transmembrane domain may be from the same protein that the signalling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect, the transmembrane domain is capable of signalling to the intracellular domain(s) whenever the CAR has bound to a target. A transmembrane domain of particular use in this disclosure may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge. In one embodiment, the hinge or spacer comprises (e.g., consists of) the amino acid sequence of SEQ ID NO: 265. In one aspect, the transmembrane domain comprises (e.g., consists of) a transmembrane domain of SEQ ID NO: 266.

In certain embodiments, the encoded transmembrane domain comprises an amino acid sequence of a CD8 transmembrane domain having at least one, two or three modifications but not more than 20, 10 or 5 modifications of the amino acid sequence of SEQ ID NO: 266, or a sequence with at least 95% identity to the amino acid sequence of SEQ ID NO: 266. In one embodiment, the encoded transmembrane domain comprises the sequence of SEQ ID NO: 266.

In other embodiments, the nucleic acid molecule encoding the CAR comprises a nucleotide sequence of a CD8 transmembrane domain, e.g., comprising the sequence of SEQ ID NO: 267 or SEQ ID NO: 304, or a sequence with at least 95% identity thereof.

In certain embodiments, the encoded antigen binding domain is connected to the transmembrane domain by a hinge region. In one embodiment, the encoded hinge region comprises the amino acid sequence of a CD8 hinge, e.g., SEQ ID NO: 265; or the amino acid sequence of an IgG4 hinge, e.g., SEQ ID NO: 268 or a sequence with at least 95% identity to SEQ ID NO: 265 or SEQ ID NO: 268. In other embodiments, the nucleic acid sequence encoding the hinge region comprises the sequence of SEQ ID NO: 269 or SEQ ID NO: 270, corresponding to a CD8 hinge or an IgG4 hinge, respectively, or a sequence with at least 95% identity to SEQ ID NO: 269 or 270.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSDGSFFL YSRLTVDKSRWQEGNVFSCSVMHEALHNHYTQKSLSLSLGKM (SEQ ID NO: 268). In some embodiments, the hinge or spacer comprises a hinge encoded by the nucleotide sequence of

(SEQ ID NO: 270)
GAGAGCAAGTACGGCCCTCCCTGCCCCCCTTGCCC
TGCCCCCGAGTTCCTGGGCGGACCCAGCGTGTTCC
TGTTCCCCCCCAAGCCCAAGGACACCCTGATGATC
AGCCGGACCCCCGAGGTGACCTGTGTGGTGGTGGA
CGTGTCCCAGGAGGACCCCGAGGTCCAGTTCAACT
GGTACGTGGACGGCGTGGAGGTGCACAACGCCAAG
ACCAAGCCCCGGGAGGAGCAGTTCAATAGCACCTA
CCGGGTGGTGTCCGTGCTGACCGTGCTGCACCAGG
ACTGGCTGAACGGCAAGGAATACAAGTGTAAGGTG
TCCAACAAGGGCCTGCCCAGCAGCATCGAGAAAAC
CATCAGCAAGGCCAAGGGCCAGCCTCGGGAGCCCC
AGGTGTACACCCTGCCCCCTAGCCAAGAGGAGATG
ACCAAGAACCAGGTGTCCCTGACCTGCCTGGTGAA
GGGCTTCTACCCCAGCGACATCGCCGTGGAGTGGG
AGAGCAACGGCCAGCCCGAGAACAACTACAAGACC
ACCCCCCCTGTGCTGGACAGCGACGGCAGCTTCTT
CCTGTACAGCCGGCTGACCGTGGACAAGAGCCGGT
GGCAGGAGGGCAACGTCTTTAGCTGCTCCGTGATG
CACGAGGCCCTGCACAACCACTACACCCAGAAGAG
CCTGAGCCTGTCCCTGGGCAAGATG.

In one aspect, the hinge or spacer comprises an IgD hinge. For example, in one embodiment, the hinge or spacer comprises a hinge of the amino acid sequence of RWPESPKAQASSVPTAQPQAEGSLAKATTAPATTRNTGRGGEEKKKEKEKEEQEERETKTPE CPSHTQPLGVYLLTPAVQDLWLRDKATFTCFVVGSDLKDAHLTWEVAGKVPTGGVEEGLLE RHSNGSQSQHSRLTLPRSLWNAGTSVTCTLNHPSLPPQRLMALREPAAQAPVKLSLNLLASS DPPEAASWLLCEVSGFSPPNILLMWLEDQREVNTSGFAPARPPPQPGSTTFWAWSVLRVPAPP SPQPATYTCVVSHEDSRTLLNASRSLEVSYVTDH (SEQ ID NO: 271). In some embodiments, the hinge or spacer comprises a hinge encoded by the nucleotide sequence of

(SEQ ID NO: 272)
AGGTGGCCCGAAAGTCCCAAGGCCCAGGCATCTAG
TGTTCCTACTGCACAGCCCCAGGCAGAAGGCAGCC
TAGCCAAAGCTACTACTGCACCTGCCACTACGCGC
AATACTGGCCGTGGCGGGGAGGAGAAGAAAAAGGA
GAAAGAGAAAGAAGAACAGGAAGAGAGGGAGACCA
AGACCCCTGAATGTCCATCCCATACCCAGCCGCTG
GGCGTCTATCTCTTGACTCCCGCAGTACAGGACTT
GTGGCTTAGAGATAAGGCCACCTTTACATGTTTCG
TCGTGGGCTCTGACCTGAAGGATGCCCATTTGACT
TGGGAGGTTGCCGGAAAGGTACCCACAGGGGGGGT
TGAGGAAGGGTTGCTGGAGCGCCATTCCAATGGCT
CTCAGAGCCAGCACTCAAGACTCACCCTTCCGAGA
TCCCTGTGGAACGCCGGGACCTCTGTCACATGTAC
TCTAAATCATCCTAGCCTGCCCCCACAGCGTCTGA
TGGCCCTTAGAGAGCCAGCCGCCCAGGCACCAGTT
AAGCTTAGCCTGAATCTGCTCGCCAGTAGTGATCC
CCCAGAGGCCGCCAGCTGGCTCTTATGCGAAGTGT
CCGGCTTTAGCCCGCCCAACATCTTGCTCATGTGG
CTGGAGGACCAGCGAGAAGTGAACACCAGCGGCTT
CGCTCCAGCCCGGCCCCCACCCCAGCCGGGTTCTA
CCACATTCTGGGCCTGGAGTGTCTTAAGGGTCCCA
GCACCACCTAGCCCCCAGCCAGCCACATACACCTG
TGTTGTGTCCCATGAAGATAGCAGGACCCTGCTAA
ATGCTTCTAGGAGTCTGGAGGTTTCCTACGTGACT
GACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO: 273). In some embodiments, the linker is encoded by the nucleotide sequence of GGTGGCGGAGGTTCTGGAGGTGGAGGTTCC (SEQ ID NO: 274).

In one aspect, the hinge or spacer comprises a KIR2DS2 hinge.

### Signalling Domains

In embodiments of the disclosure having an intracellular signalling domain, such a domain can contain, e.g., one or more of a primary signalling domain and/or a costimulatory signalling domain. In some embodiments, the intracellular signalling domain comprises a sequence encoding a primary signalling domain. In some embodiments, the intracellular signalling domain comprises a costimulatory signalling domain. In some embodiments, the intracellular signalling domain comprises a primary signalling domain and a costimulatory signalling domain.

The intracellular signalling sequences within the cytoplasmic portion of the CAR of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signalling sequences. In one embodiment, a glycine-serine doublet can be used as a suitable linker. In one embodiment, a single amino acid, e.g., an alanine, a glycine, can be used as a suitable linker.

In one aspect, the intracellular signalling domain is designed to comprise two or more, e.g., 2, 3, 4, 5, or more, costimulatory signalling domains. In an embodiment, the two or more, e.g., 2, 3, 4, 5, or more, costimulatory signalling domains, are separated by a linker molecule, e.g., a linker molecule described herein. In one embodiment, the intracellular signalling domain comprises two costimulatory signalling domains. In some embodiments, the linker molecule is a glycine residue. In some embodiments, the linker is an alanine residue.

### Primary Signalling Domains

A primary signalling domain regulates primary activation of the TCR complex either in a stimulatory way, or in an inhibitory way. Primary intracellular signalling domains that act in a stimulatory manner may contain signalling motifs, which are known as immunoreceptor tyrosine-based activation motifs or ITAMs.

Examples of ITAM containing primary intracellular signalling domains that are of particular use in the disclosure include those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, a CAR of the disclosure comprises an intracellular signalling domain, e.g., a primary signalling domain of CD3-zeta.

In one embodiment, the encoded primary signalling domain comprises a functional signalling domain of CD3 zeta. The encoded CD3 zeta primary signalling domain can comprise an amino acid sequence having at least one, two or three modifications but not more than 20, 10 or 5 modifications of the amino acid sequence of SEQ ID NO: 275 or SEQ ID NO: 276, or a sequence with at least 95% identity to the amino acid sequence of SEQ ID NO: 275 or SEQ ID NO: 276. In some embodiments, the encoded primary signalling domain comprises the sequence of SEQ ID NO: 275 or SEQ ID NO: 276. In other embodiments, the nucleic acid sequence encoding the primary signalling domain comprises the sequence of SEQ ID NO: 277, SEQ ID NO: 303, or SEQ ID NO: 278, or a sequence with at least 95% identity thereof.

### Costimulatory Signalling Domains

In some embodiments, the encoded intracellular signalling domain comprises a costimulatory signalling domain. For example, the intracellular signalling domain can comprise a primary signalling domain and a costimulatory signalling domain. In some embodiments, the encoded costimulatory signalling domain comprises a functional signalling domain of a protein selected from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In certain embodiments, the encoded costimulatory signalling domain comprises an amino acid sequence having at least one, two or three modifications but not more than 20, 10 or 5 modifications of the amino acid sequence of SEQ ID NO: 279 or SEQ ID NO: 280, or a sequence with at least 95% identity to the amino acid sequence of SEQ ID NO: 279 or SEQ ID NO: 280. In one embodiment, the encoded costimulatory signalling domain comprises the sequence of SEQ ID NO: 279 or SEQ ID NO: 280. In other embodiments, the nucleic acid sequence encoding the costimulatory signalling domain comprises the sequence of SEQ ID NO: 281, SEQ ID NO: 305, or SEQ ID NO: 282, or a sequence with at least 95% identity thereof.

In other embodiments, the encoded intracellular domain comprises the sequence of SEQ ID NO: 279 or SEQ ID NO: 280 and the sequence of SEQ ID NO: 275 or SEQ ID NO: 276, wherein the sequences comprising the intracellular signalling domain are expressed in the same frame and as a single polypeptide chain.

In certain embodiments, the nucleic acid sequence encoding the intracellular signalling domain comprises the sequence of SEQ ID NO: 281, SEQ ID NO: 305, or SEQ ID NO: 282, or a sequence with at least 95% identity thereof, and the sequence of SEQ ID NO: 277, SEQ ID NO: 306, or SEQ ID NO: 278, or a sequence with at least 95% identity thereof.

In some embodiments, the nucleic acid molecule further encodes a leader sequence. In one embodiment, the leader sequence comprises the sequence of SEQ ID NO: 283.

In one aspect, the intracellular signalling domain is designed to comprise the signalling domain of CD3-zeta and the signalling domain of CD28. In one aspect, the intracellular signalling domain is designed to comprise the signalling domain of CD3-zeta and the signalling domain of 4-1BB. In one aspect, the signalling domain of 4-1BB is a signalling domain of SEQ ID NO: 279. In one aspect, the signalling domain of CD3-zeta is a signalling domain of SEQ ID NO: 275.

In one aspect, the intracellular signalling domain is designed to comprise the signalling domain of CD3-zeta and the signalling domain of CD27. In one aspect, the signalling domain of CD27 comprises the amino acid sequence of QRRKYRSNKGESPVEPAEPCRYSCPREEEGSTIPIQEDYRKPEPACSP (SEQ ID NO: 280). In one aspect, the signalling domain of CD27 is encoded by the nucleic acid sequence of

(SEQ ID NO: 282)
CAACGAAGGAAATATAGATCAAACAAAGGAGAAAG
TCCTGTGGAGCCTGCAGAGCCTTGTCGTTACAGCT
GCCCCAGGGAGGAGGAGGGCAGCACCATCCCCATC
CAGGAGGATTACCGAAAACCGGAGCCTGCCTGCTC
CCCC.

### Vectors

In another aspect, the disclosure pertains to a vector comprising a nucleic acid sequence encoding a CAR described herein. In one embodiment, the vector is selected from a DNA vector, an RNA vector, a plasmid, a lentivirus vector, adenoviral vector, or a retrovirus vector. In one embodiment, the vector is a lentivirus vector. These vectors or portions thereof may, among other things, be used to create template nucleic acids, as described herein for use with the CRISPR systems as described herein. Alternatively, the vectors may be used to deliver nucleic acid directly to the cell, e.g., the immune effector cell, e.g., the T cell, e.g., the allogeneic T cell, independent of the CRISPR system.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the disclosure is an adenoviral vector (A5/35). In another embodiment, the expression of nucleic acids encoding CARs can be accomplished using of transposons such as sleeping beauty, crisper, CAS9, and zinc finger nucleases. See below June et al. 2009 Nature Reviews Immunology 9.10: 704-716.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Disclosed herein are methods for producing an in vitro transcribed RNA CAR. The present disclosure also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO: 310). RNA so produced can efficiently transfect different kinds of cells. In one aspect, the template includes sequences for the CAR.

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, cells of the disclosure, e.g., T or NK cells, e.g., allogeneic T cells, e.g., described herein, (e.g., that express a CAR described herein) are generated by contacting the cells with (a) a composition comprising one or more gRNA molecules, e.g., as described herein, and one or more Cas molecules, e.g., a Cas9 molecule, e.g., as described herein, and (b) nucleic acid comprising sequence encoding a CAR, e.g., described herein (such as a template nucleic acid molecule as described herein). Without being bound by theory, said composition of (a), above, will induce a break at or near the genomic DNA targeted by the targeting domain of the gRNA molecule(s), and the nucleic acid of (b) will incorporate, e.g., partially or wholly, into the genome at or near said break, such that upon integration, the encoded CAR molecule is expressed. In embodiments, expression of the CAR will be controlled by promoters or other regulatory elements endogenous to the genome (e.g., the promoter controlling expression from the gene in which the nucleic acid of (b) was inserted). In other embodiments, the nucleic acid of (b) further comprises a promoter and/or other regulatory elements, e.g., as described herein, e.g., an EF1-alpha promoter, operably linked to the sequence encoding the CAR, such that upon integration, expression of the CAR is controlled by that promoter and/or other regulatory elements. Additional features of the disclosure relating to use of CRISPR/Cas9 systems, e.g., as described herein, to direct incorporation of nucleic acid sequence encoding a CAR, e.g., as described herein, are described elsewhere in this application, e.g., in the section relating to gene insertion and homologous recombination. In embodiments, the composition of a) above is a composition comprising RNPs comprising the one or more gRNA molecules. In embodiments, RNPs comprising gRNAs targeting unique target sequences are introduced into the cell simultaneously, e.g., as a mixture of RNPs comprising the one or more gRNAs. In embodiments, RNPs comprising gRNAs targeting unique target sequences are introduced into the cell sequentially.

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Promoters

In one embodiment, the vector further comprises a promoter. In some embodiments, the promoter is selected from an EF-1 promoter, a CMV IE gene promoter, an EF-1α promoter, an ubiquitin C promoter, or a phosphoglycerate kinase (PGK) promoter. In one embodiment, the promoter is an EF-1 promoter. In one embodiment, the EF-1 promoter comprises the sequence of SEQ ID NO: 285.

### Host Cells for CAR Expression

As noted above, in some aspects the disclosure pertains to a cell, e.g., an immune effector cell, (e.g., a population of cells, e.g., a population of immune effector cells) comprising a nucleic acid molecule, a CAR polypeptide molecule, or a vector as described herein.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.20140.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

In some aspects, the cells of the disclosure (e.g., the immune effector cells of the disclosure, e.g., the CAR-expressing cells of the disclosure) are induced pluripotent stem cells (“iPSCs”) or embryonic stem cells (ESCs), or are T cells generated from (e.g., differentiated from) said iPSC and/or ESC. iPSCs can be generated, for example, by methods known in the art, from peripheral blood T lymphocytes, e.g., peripheral blood T lymphocytes isolated from a healthy volunteer. As well, such cells may be differentiated into T cells by methods known in the art. See e.g., Themeli M. et al., Nat. Biotechnol., 31, pp. 928-933 (2013); doi:10.1038/nbt.2678; WO2014/165707.

In another embodiment, TGFβ inhibitors (and/or PD1, PD-L1, or PD-L2 inhibitor) of the present disclosure are used in combination with one or more of the therapeutic agents listed in Table 13 or listed in the patent and patent applications cited in Table 13, to treat cancer. Each publication listed in Table 13, including all structural formulae therein.

TABLE 13

| Second | | | Patents/Patent |
|---|---|---|---|
| agent | Generic Name | | Application |
| No. | Tradename | Compound Structure | Publications |
| A1 | Sotrastaurin | 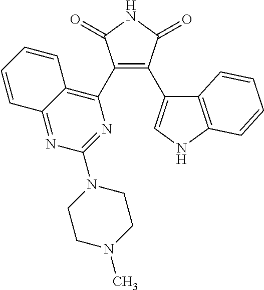 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| A2 | Nilotinib HCl monohydrate TASIGNA ® | 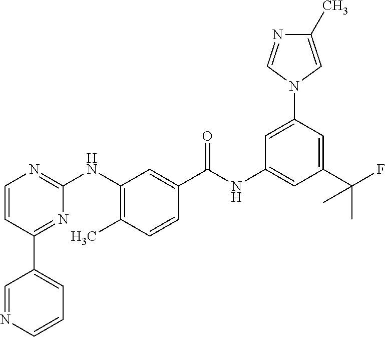 [SMILES: Cc1cn(-c2cc(NC(=O)c3ccc(C)c(Nc4nccc(-c5cccnc5)n4)c3)cc(C(C)(C)F)c2)cn1] HCl•H₂O | WO 2004/005281 U.S. Pat. No. 7,169,791 |
| A3 | | 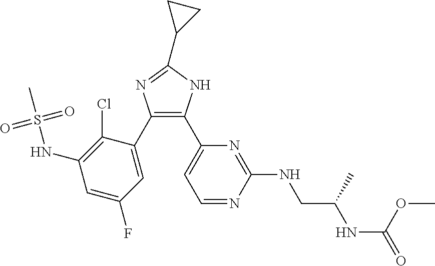 [SMILES: COC(=O)N[C@@H](C)CNc1nccc(-c2[nH]c(C3CC3)nc2-c2cc(F)cc(NS(C)(=O)=O)c2Cl)n1] | WO 2011/023773 |
| A4 | | 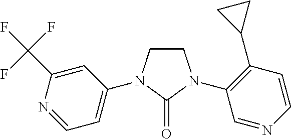 [SMILES: O=C1N(c2ccnc(C(F)(F)F)c2)CCN1c1cnccc1C1CC1] | WO 2012/149413 |
| A6 | | 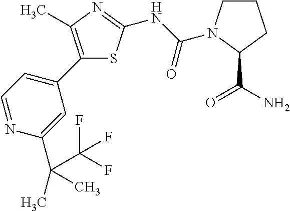 [SMILES: Cc1nc(NC(=O)N2CCC[C@H]2C(N)=O)sc1-c1ccnc(C(C)(C)C(F)(F)F)c1] | WO 2010/029082 |
| A7 | | 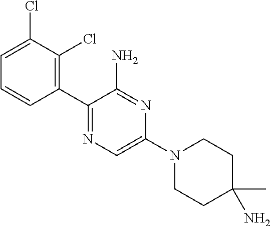 [SMILES: CC1(N)CCN(c2cnc(-c3cccc(Cl)c3Cl)c(N)n2)CC1] | WO 2015/107493 |
| A8 | | | WO 2015/107495 |
| A9 | | 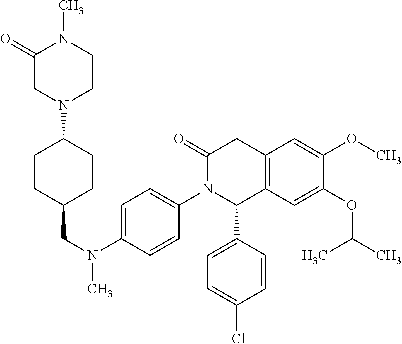 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A10 | Deferasirox EXJADE ® | 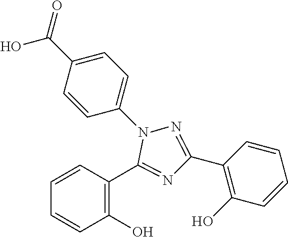 [SMILES: O=C(O)c1ccc(-n2nc(-c3ccccc3O)nc2-c2ccccc2O)cc1] | WO 1997/049395 |
| A11 | Letrozole FEMARA ® | 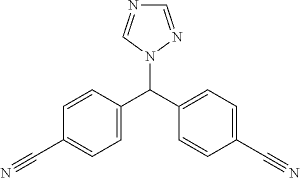 [SMILES: N#Cc1ccc(C(c2ccc(C#N)cc2)n2cncn2)cc1] | U.S. Pat. No. 4,978,672 |
| A12 | | 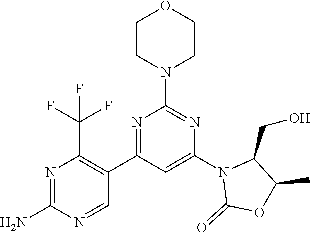 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A13 | | 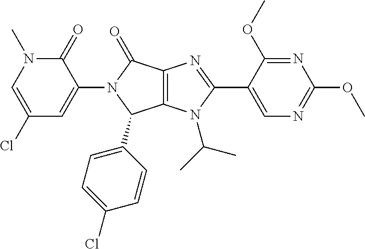 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A14 | | 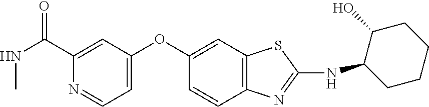 [SMILES: CNC(=O)c1cc(Oc2ccc3nc(N[C@@H]4CCCC[C@H]4O)sc3c2)ccn1] | WO 2007/121484 |
| A15 | Imatinib mesylate GLEEVEC ® | 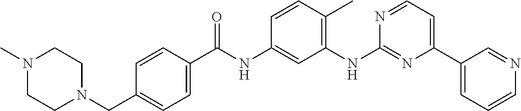 [SMILES: Cc1ccc(NC(=O)c2ccc(CN3CCN(C)CC3)cc2)cc1Nc1nccc(-c2cccnc2)n1] Mesylate | WO 1999/003854 |
| A16 | Capmatinib | 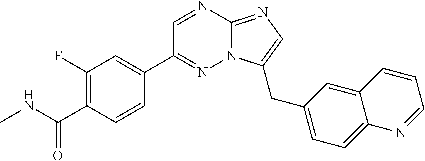 [SMILES: CNC(=O)c1ccc(-c2cnc3ncc(Cc4ccc5ncccc5c4)n3n2)cc1F] Dihydrochloric salt | EP 2099447 U.S. Pat. No. 7,767,675 U.S. Pat. No. 8,420,645 |
| A17 | Ruxolitinib Phosphate JAKAFI ® | 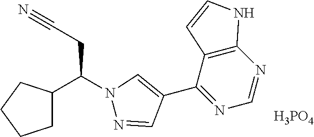 | WO 2007/070514; EP 2474545 U.S. Pat. No. 7,598,257; WO 2014/018632 |
| A18 | Panobinostat | 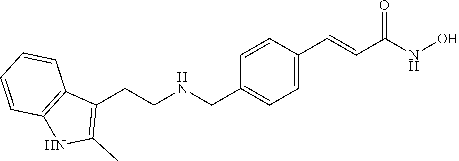 [SMILES: Cc1[nH]c2ccccc2c1CCNCc1ccc(/C=C/C(=O)NO)cc1] | WO 2014/072493 WO 2002/022577 EP 1870399 |
| A20 | | 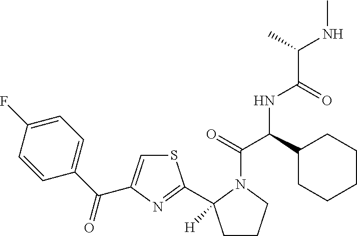 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 U.S. Pat. No. 8,552,003 |
| A21 | | 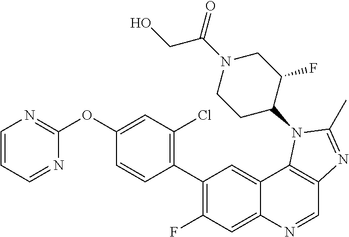 | WO 2015/022662 |
| A22 | ceritinib ZYKADIA ™ | 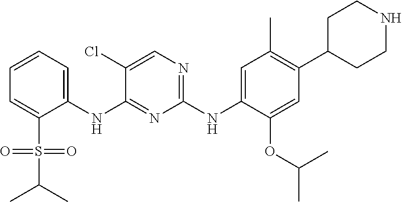 [SMILES: Cc1cc(Nc2ncc(Cl)c(Nc3ccccc3S(=O)(=O)C(C)C)n2)c(OC(C)C)cc1C1CCNCC1] | WO 2008/073687 U.S. Pat. No. 8,039,479 |
| A23 | Ribociclib KISQALI ® | 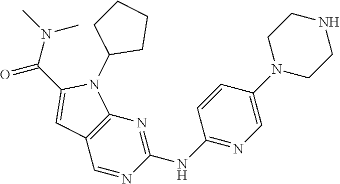 [SMILES: CN(C)C(=O)c1cc2cnc(Nc3ccc(N4CCNCC4)cn3)nc2n1C1CCCC1] | U.S. Pat. No. 8,415,355 U.S. Pat. No. 8,685,980 |
| A24 | | 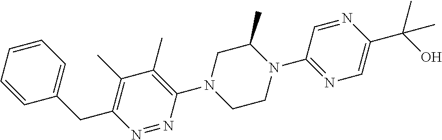 [SMILES: Cc1c(Cc2ccccc2)nnc(N2CCN(c3cnc(C(C)(C)O)cn3)[C@H](C)C2)c1C] | WO 2010/007120 |
| A26 | | 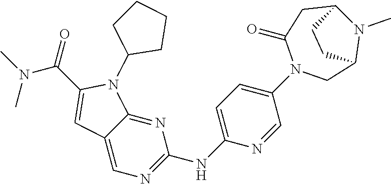 | WO 2011/101409 |
| A27 | | Human monoclonal antibody to HER3 | WO 2012/022814 |
| | | | EP 2606070 |
| | | | U.S. Pat. No. |
| | | | 8,735,551 |
| A28 | | Antibody Drug Conjugate (ADC) | WO 2014/160160 |
| A29 | | Monoclonal antibody or Fab to M-CSF | WO 2004/045532 |
| A30 | Midostaurin | 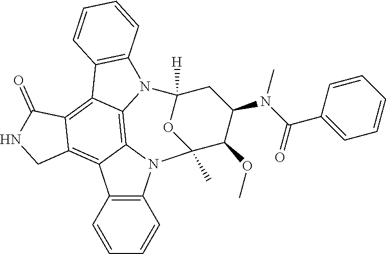 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347 EP 1441737 US 2012/252785 |
| A31 | Everolimus AFINITOR ® | 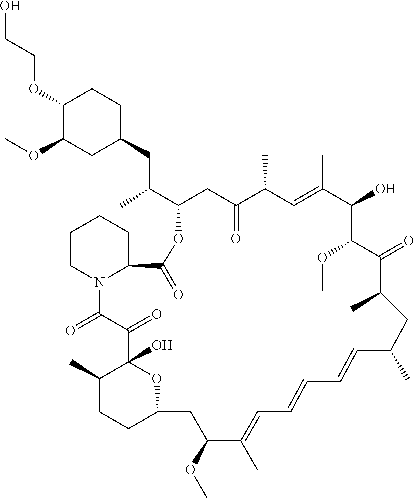 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | WO 1994/009010 WO 2014/085318 |
| A32 | | 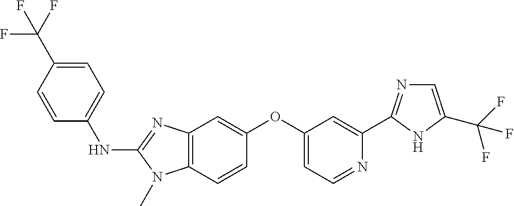 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| A34 | | 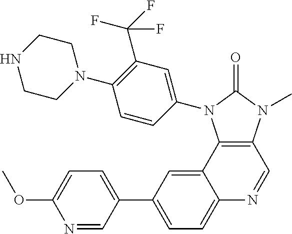 | WO 2006/122806 |
| A35 | | 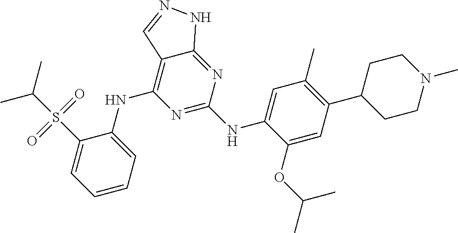 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| A36 | Valspodar AMDRAY ™ | 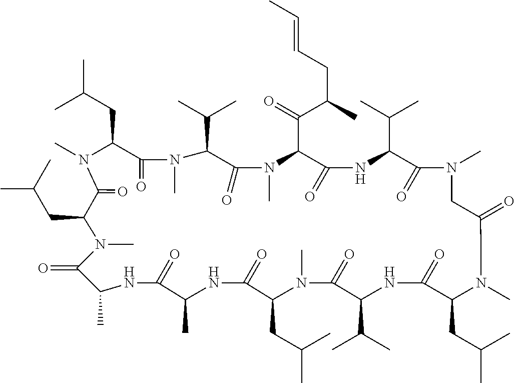 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A37 | Vatalanib succinate | 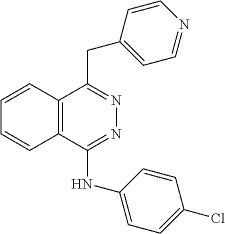 [SMILES: Clc1ccc(Nc2nnc(Cc3ccncc3)c3ccccc23)cc1] succinate | WO 98/35958 |
| A38 | | 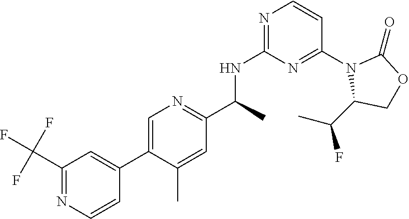 [SMILES: Cc1cc([C@H](C)Nc2nccc(N3C(=O)OC[C@@H]3[C@H](C)F)n2)ncc1-c1ccnc(C(F)(F)F)c1] | WO 2014/141104 |
| A39 | Asciminib | 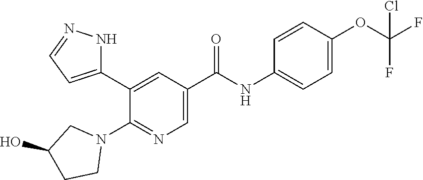 [SMILES: O=C(Nc1ccc(OC(F)(F)Cl)cc1)c1cnc(N2CC[C@@H](O)C2)c(-c2ccn[nH]2)c1] | WO 2013/171639 WO 2013/171640 WO 2013/171641 WO 2013/171642 |
| A42 | | 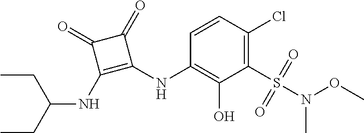 [SMILES: CCC(CC)Nc1c(Nc2ccc(Cl)c(S(=O)(=O)N(C)OC)c2O)c(=O)c1=O] or a choline salt thereof | WO 2010/015613 WO 2013030803 U.S. Pat. No. 7,989,497, |
| A43 | | | WO 2017/025918 |
| | | | WO 2011/121418 |
| | | | U.S. Pat. No. |
| | | | 8,796,284 |
| A44 | | 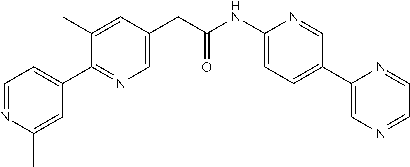 [SMILES: Cc1cc(-c2ncc(CC(=O)Nc3ccc(-c4cnccn4)cn3)cc2C)ccn1] | WO 2010/101849 |
| A45 | | 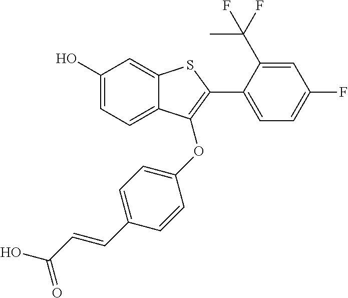 [SMILES: CC(F)(F)c1cc(F)ccc1-c1sc2cc(O)ccc2c1Oc1ccc(/C=C/C(=O)O)cc1] | WO 2014/130310 |
| A46 | trametinib | 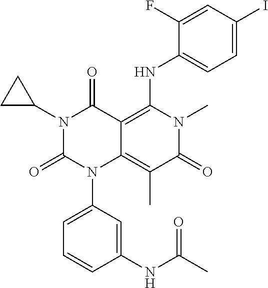 [SMILES: CC(=O)Nc1cccc(-n2c(=O)n(C3CC3)c(=O)c3c(Nc4ccc(I)cc4F)n(C)c(=O)c(C)c32)c1] | WO 2005/121142 U.S. Pat. No. 7,378,423 |
| A47 | dabrafenib | 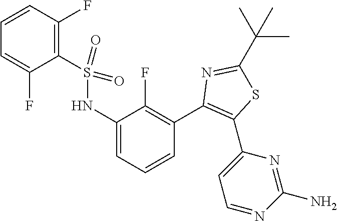 [SMILES: CC(C)(C)c1nc(-c2cccc(NS(=O)(=O)c3c(F)cccc3F)c2F)c(-c2ccnc(N)n2)s1] | WO 2009/137391 U.S. Pat. No. 7,994,185 |
| A49 | octreotide | 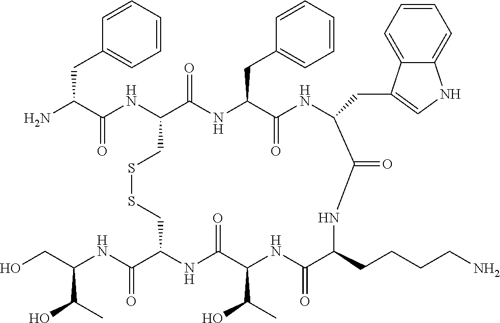 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O] | U.S. Pat. No. 4,395,403 EP 0 029 579 |
| A50 | | 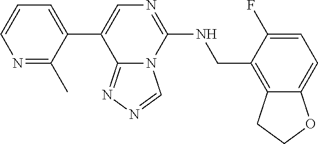 [SMILES: Cc1ncccc1-c1cnc(NCc2c(F)ccc3c2CCO3)n2cnnc12] | WO 2016/103155 U.S. Pat. No. 9580437 EP 3237418 |
| A51 | | 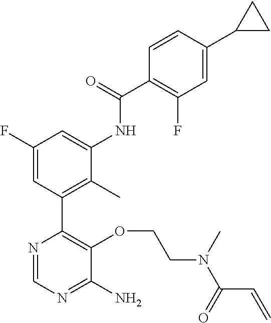 [SMILES: C=CC(=O)N(C)CCOc1c(N)ncnc1-c1cc(F)cc(NC(=O)c2ccc(C3CC3)cc2F)c1C] | U.S. Pat. No. 9,512,084 WO/2015/079417 |
| A52 | | 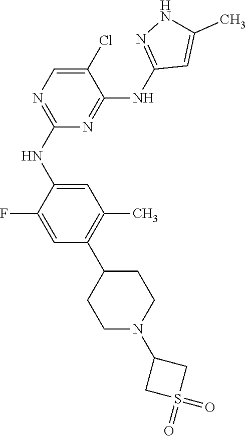 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| A53 | | 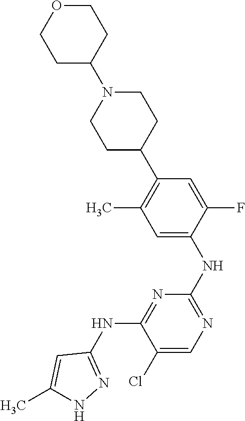 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| A54 | | 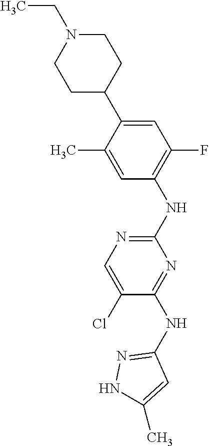 [SMILES: CCN1CCC(c2cc(F)c(Nc3ncc(Cl)c(Nc4cc(C)[nH]n4)n3)cc2C)CC1] | WO 2010/002655 |

### Estrogen Receptor Antagonists

In some embodiments, an estrogen receptor (ER) antagonist is used in combination with TGFβ inhibitors (and/or PD1, PD-L1, or PD-L2 inhibitor), for treating a disease, e.g., cancer. In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signalling is implicated in cancers (e.g., breast cancer), e.g., ER positive (ER+) breast cancer. In some embodiments, the SERD is selected from LSZ102, fulvestrant, brilanestrant, or elacestrant.

### Exemplary Estrogen Receptor Antagonists

In some embodiments, the SERD comprises a compound disclosed in International Application Publication No. WO 2014/130310. In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid.

### Other Exemplary Estrogen Receptor Antagonists

In some embodiments, the SERD comprises fulvestrant (CAS Registry Number: 129453-61-8), or a compound disclosed in International Application Publication No. WO 2001/051056. Fulvestrant is also known as ICI 182780, ZM 182780, FASLODEX®, or (7α,17β)-7-{9-[(4,4,5,5,5-pentafluoropentyl)sulfinyl]nonyl}estra-1,3,5(10)-triene-3,17-diol. Fulvestrant is a high affinity estrogen receptor antagonist with an IC50 of 0.29 nM.

In some embodiments, the SERD comprises elacestrant (CAS Registry Number: 722533-56-4), or a compound disclosed in U.S. Pat. No. 7,612,114. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56.

In some embodiments, the SERD is brilanestrant (CAS Registry Number: 1365888-06-7), or a compound disclosed in International Application Publication No. WO 2015/136017. Brilanestrant is also known as GDC-0810, ARN810, RG-6046, RO-7056118 or (2E)-3-{4-[(1E)-2-(2-chloro-4-fluorophenyl)-1-(1H-indazol-5-yl)but-1-en-1-yl]phenyl}prop-2-enoic acid. Brilanestrant is a next-generation, orally bioavailable selective SERD with an IC50 of 0.7 nM. Brilanestrant is also disclosed, e.g., in Lai A. et al. (2015) Journal of Medicinal Chemistry 58 (12): 4888-4904.

In some embodiments, the SERD is selected from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887. Other exemplary estrogen receptor antagonists are disclosed, e.g., in WO 2011/156518, WO 2011/159769, WO 2012/037410, WO 2012/037411, and US 2012/0071535.

### CDK4/6 Inhibitors

In some embodiments, an inhibitor of Cyclin-Dependent Kinases 4 or 6 (CDK4/6) is used in combination with TGFβ inhibitors (and/or PD1, PD-L1, or PD-L2 inhibitor), for treating a disease, e.g., cancer. In some embodiments, the CDK4/6 inhibitor is selected from ribociclib, abemaciclib (Eli Lilly), or palbociclib.

### Exemplary CDK4/6 Inhibitors

In some embodiments, the CDK4/6 inhibitor comprises ribociclib (CAS Registry Number: 1211441-98-3), or a compound disclosed in U.S. Pat. Nos. 8,415,355 and 8,685,980.

In some embodiments, the CDK4/6 inhibitor comprises a compound disclosed in International Application Publication No. WO 2010/020675 and U.S. Pat. Nos. 8,415,355 and 8,685,980.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

### Other Exemplary CDK4/6 Inhibitors

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

### CXCR2 Inhibitors

In some embodiments, an inhibitor of chemokine (C—X—C motif) receptor 2 (CXCR2) is used in combination with TGFβ inhibitors (and/or PD1, PD-L1, or PD-L2 inhibitor), for treating a disease, e.g., cancer. In some embodiments, the CXCR2 inhibitor is selected from 6-chloro-3-((3,4-dioxo-2-(pentan-3-ylamino)cyclobut-1-en-1-yl)amino)-2-hydroxy-N-methoxy-N-methylbenzene sulfonamide, danirixin, reparixin, or navarixin.

### Exemplary CXCR2 Inhibitors

In some embodiments, the CXCR2 inhibitor comprises a compound disclosed in U.S. Pat. Nos. 7,989,497, 8,288,588, 8,329,754, 8,722,925, 9,115,087, U.S. Application Publication Nos. US 2010/0152205, US 2011/0251205 and US 2011/0251206, and International Application Publication Nos. WO 2008/061740, WO 2008/061741, WO 2008/062026, WO 2009/106539, WO2010/063802, WO 2012/062713, WO 2013/168108, WO 2010/015613 and WO 2013/030803. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt) and has the following chemical structure:

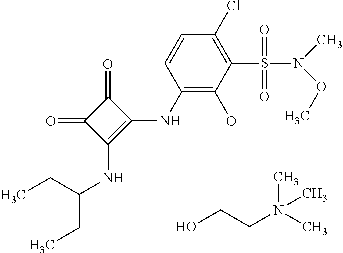

### Other Exemplary CXCR2 Inhibitors

In some embodiments, the CXCR2 inhibitor comprises danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18.

In some embodiments, the CXCR2 inhibitor comprises reparixin (CAS Registry Number: 266359-83-5). Reparixin is also known as repertaxin or (2R)-2-[4-(2-methylpropyl)phenyl]-N-methylsulfonylpropanamide Reparixin is a non-competitive allosteric inhibitor of CXCR1/2. Reparixin is disclosed, e.g., in Zarbock et al. Br J Pharmacol. 2008; 155(3):357-64.

In some embodiments, the CXCR2 inhibitor comprises navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide. Navarixin is disclosed, e.g., in Ning et al. Mol Cancer Ther. 2012; 11(6):1353-64.

### CSF-1/1R Binding Agents

In some embodiments, a CSF-1/1R binding agent is used in combination with TGFβ inhibitors (and/or PD1, PD-L1, or PD-L2 inhibitor), for treating a disease, e.g., cancer. In some embodiments, the CSF-1/1R binding agent is selected from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

### Exemplary CSF-1 Binding Agents

In some embodiments, the CSF-1/1R binding agent comprises an inhibitor of macrophage colony-stimulating factor (M-CSF). M-CSF is also sometimes known as CSF-1. In certain embodiments, the CSF-1/1R binding agent is an antibody to CSF-1 (e.g., MCS110). In other embodiments, the CSF-1/1R binding agent is an inhibitor of CSF-1R (e.g., BLZ945).

In some embodiments, the CSF-1/1R binding agent comprises a monoclonal antibody or Fab to M-CSF (e.g., MCS110/H-RX1), or a binding agent to CSF-1 disclosed in International Application Publication Nos. WO 2004/045532 and WO 2005/068503, including H-RX1 or 5H4 (e.g., an antibody molecule or Fab fragment against M-CSF) and U.S. Pat. No. 9,079,956.

TABLE 13a
Amino acid and nucleotide sequences
of an exemplary anti-M-CSF antibody
molecule (MCS110)

| | |
|---|---|
| (H-RXI) HC | QVQLQESGPGLVKPSQTLSL |
| | TCTVSDYSITSDYAWNWIRQ |
| | FPGKGLEWMGYISYSGSTSY |
| | NPSLKSRITISRDTSKNQFS |
| | LQLNSVTAADTAVYYCASFD |
| | YAHAMDYWGQGTTVTVSSAS |
| | TKGPSVFPLAPSSKSTSGGT |
| | AALGCLVKDYFPEPVTVSWN |
| | SGALTSGVHTFPAVLQSSGL |
| | YSLSSVVTVPSSSLGTQTYI |
| | CNVNHKPSNTKVDKRVEPKS |
| | CDKTHTCPPCPAPELLGGPS |
| | VFLFPPKPKDTLMISRTPEV |
| | TCVVVDVSHEDPEVKFNWYV |
| | DGVEVHNAKTKPREEQYNST |
| | YRVVSVLTVLHQDWLNGKEY |
| | KCKVSNKALPAPIEKTISKA |
| | KGQPREPQVYTLPPSREEMT |
| | KNQVSLTCLVKGFYPSDIAV |
| | EWESNGQPENNYKTTPPVLD |
| | SDGSFFLYSKLTVDKSRWQQ |
| | GNVFSCSVMHEALHNHYTQK |
| | SLSLSPGK |
| | (SEQ ID NO: 286) |
| (H-RXI) LC | DIVLTQSPAFLSVTPGEKVT |
| | FTCQASQSIGTSIHWYQQKT |
| | DQAPKLLIKYASESISGIPS |
| | RFSGSGSGTDFTLTISSVEA |
| | EDAADYYCQQINSWPTTFGG |
| | GTKLEIKRTVAAPSVFIFPP |
| | SDEQLKSGTASVVCLLNNFY |
| | PREAKVQWKVDNALQSGNSQ |
| | ESVTEQDSKDSTYSLSSTLT |
| | LSKADYEKHKVYACEVTHQG |
| | LSSPVTKSFNRGEC |
| | (SEQ ID NO: 287) |
| Heavy Chain | SDYAWN |
| CDR1 (Kabat) | (SEQ ID NO: 288) |
| Heavy Chain | YISYSGSTSYNPSLKS |
| CDR2 (Kabat) | (SEQ ID NO: 289) |
| Heavy Chain | FDYAHAMDY |
| CDR3 (Kabat) | (SEQ ID NO: 290) |
| Light Chain | QASQSIGTSIH |
| CDRI (Kabat) | (SEQ ID NO: 291) |
| Light Chain | YASESIS |
| CDR2 (Kabat) | (SEQ ID NO: 292) |
| Light Chain | QQINSWPTT |
| CDR3 (Kabat) | (SEQ ID NO: 293) |

In another embodiment, the CSF-1/1R binding agent comprises a CSF-1R tyrosine kinase inhibitor, 4-((2-(((1R,2R)-2-hydroxycyclohexyl)amino)benzo[d]thiazol-6-yl)oxy)-N-methylpicolinamide (BLZ945), or a compound disclosed in International Application Publication No. WO 2007/121484, and U.S. Pat. Nos. 7,553,854, 8,173,689, and 8,710,048.

### Other Exemplary CSF-1/1R Binding Agents

In some embodiments, the CSF-1/1R binding agent comprises pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine. Pexidartinib is a small-molecule receptor tyrosine kinase (RTK) inhibitor of KIT, CSF1R and FLT3. FLT3, CSF1R and FLT3 are overexpressed or mutated in many cancer cell types and play major roles in tumor cell proliferation and metastasis. PLX3397 can bind to and inhibit phosphorylation of stem cell factor receptor (KIT), colony-stimulating factor-1 receptor (CSF1R) and FMS-like tyrosine kinase 3 (FLT3), which may result in the inhibition of tumor cell proliferation and down-modulation of macrophages, osteoclasts and mast cells involved in the osteolytic metastatic disease.

In some embodiments, the CSF-1/1R binding agent is emactuzumab. Emactuzumab is also known as RG7155 or R05509554. Emactuzumab is a humanized IgG1 mAb targeting CSF1R. In some embodiments, the CSF-1/1R binding agent is FPA008. FPA008 is a humanized mAb that inhibits CSF1R.

### A2aR Antagonists

In some embodiments, an adenosine A2a receptor (A2aR) antagonist (e.g., an inhibitor of A2aR pathway, e.g., an adenosine inhibitor, e.g., an inhibitor of A2aR or CD-73) is used in combination with TGFβ inhibitors (and/or PD1, PD-L1, or PD-L2 inhibitor), for treating a disease, e.g., cancer. In some embodiments, the A2aR antagonist is selected from PBF509 (NIR178) (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

### Exemplary A2aR Antagonists

In some embodiments, the A2aR antagonist comprises PBF509 (NIR178) or a compound disclosed in U.S. Pat. No. 8,796,284 or in International Application Publication No. WO 2017/025918. PBF509 (NIR178) is also known as NIR178.

### Other Exemplary A2aR Antagonists

In certain embodiments, the A2aR antagonist comprises CPI444/V81444. CPI-444 and other A2aR antagonists are disclosed in International Application Publication No. WO 2009/156737. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine.

In certain embodiments, the A2aR antagonist is AZD4635/HTL-1071. A2aR antagonists are disclosed in International Application Publication No. WO 2011/095625. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197. In certain embodiments, the A2aR antagonist is an A2aR antagonist described in U.S. Pat. Nos. 8,114,845 and 9,029,393, U.S. Application Publication Nos. 2017/0015758 and 2016/0129108.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[1-[4-(2-methoxyethoxy)phenyl]-1-piperazinyl]ethyl]7H-pyrazolo[4,3-e][1,2,4]triazolo[1,5-c]pyrimidine-5-amine. Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine. Other exemplary A2aR antagonists include, e.g., ATL-444, MSX-3, SCH-58261, SCH-412,348, SCH-442,416, VER-6623, VER-6947, VER-7835, CGS-15943, and ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73. Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment. These changes include, e.g., increases in CD8 effector cells and activated macrophages, as well as a reduction in the proportions of myeloid-derived suppressor cells (MDSC) and regulatory T lymphocytes.

### IDO Inhibitors

In some embodiments, an inhibitor of indoleamine 2,3-dioxygenase (IDO) and/or tryptophan 2,3-dioxygenase (TDO) is used in combination with TGFβ inhibitors (and/or PD1, PD-L1, or PD-L2 inhibitor), for treating a disease, e.g., cancer. In some embodiments, the IDO inhibitor is selected from (4E)-4-[(3-chloro-4-fluoroanilino)-nitrosomethylidene]-1,2,5-oxadiazol-3-amine (also known as epacadostat or INCB24360), indoximod ( ), (1-methyl-D-tryptophan), α-cyclohexyl-5H-Imidazo[5,1-a]isoindole-5-ethanol (also known as NLG919), indoximod, and BMS-986205 (formerly F001287).

### Exemplary IDO Inhibitors

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### STING Agonists

In some embodiments, a STING agonist is used in combination with TGFβ inhibitors (and/or PD1, PD-L1, or PD-L2 inhibitor), for treating a disease, e.g., cancer. In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is MK-1454 (Merck). MK-1454 is a cyclic dinucleotide Stimulator of Interferon Genes (STING) agonist that activates the STING pathway. Exemplary STING agonist are disclosed, e.g., in PCT Publication No. WO 2017/027645.

### Galectin Inhibitors

In some embodiments, a Galectin, e.g., Galectin-1 or Galectin-3, inhibitor is used in combination with TGFβ inhibitors (and/or PD1, PD-L1, or PD-L2 inhibitor), for treating a disease, e.g., cancer. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is selected from an anti-Galectin antibody molecule, GR-MD-02 (Galectin Therapeutics), Galectin-3C (Mandal Med), Anginex, or OTX-008 (OncoEthix, Merck). Galectins are a family of proteins that bind to beta galactosidase sugars.

The Galectin family of proteins comprises at least of Galectin-1, Galectin-2, Galectin-3, Galectin-4, Galectin-7, and Galectin-8. Galectins are also referred to as S-type lectins, and are soluble proteins with, e.g., intracellular and extracellular functions.

Galectin-1 and Galectin-3 are highly expressed in various tumor types. Galectin-1 and Galectin-3 can promote angiogenesis and/or reprogram myeloid cells toward a pro-tumor phenotype, e.g., enhance immunosuppression from myeloid cells. Soluble Galectin-3 can also bind to and/or inactivate infiltrating T cells.

### Exemplary Galectin Inhibitors

In some embodiments, a Galectin inhibitor is an antibody molecule. In an embodiment, an antibody molecule is a monospecific antibody molecule and binds a single epitope. E.g., a monospecific antibody molecule having a plurality of immunoglobulin variable domain sequences, each of which binds the same epitope. In an embodiment, the Galectin inhibitor is an anti-Galectin, e.g., anti-Galectin-1 or anti-Galectin-3, antibody molecule. In some embodiments, the Galectin inhibitor is an anti-Galectin-1 antibody molecule. In some embodiments, the Galectin inhibitor is an anti-Galectin-3 antibody molecule.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

In some embodiments, a Galectin inhibitor is a peptide, e.g., protein, which can bind to, and inhibit Galectin, e.g., Galectin-1 or Galectin-3, function. In some embodiments, the Galectin inhibitor is a peptide which can bind to, and inhibit Galectin-3 function. In some embodiments, the Galectin inhibitor is the peptide Galectin-3C. In some embodiments, the Galectin inhibitor is a Galectin-3 inhibitor disclosed in U.S. Pat. No. 6,770,622.

Galectin-3C is an N-terminal truncated protein of Galectin-3, and functions, e.g., as a competitive inhibitor of Galectin-3. Galectin-3C prevents binding of endogenous Galectin-3 to e.g., laminin on the surface of, e.g., cancer cells, and other beta-galactosidase glycoconjugates in the extracellular matrix (ECM). Galectin-3C and other exemplary Galectin inhibiting peptides are disclosed in U.S. Pat. No. 6,770,622.

In some embodiments, Galectin-3C comprises the amino acid sequence of SEQ ID NO: 294, or an amino acid substantially identical (e.g., 90, 95 or 99%) identical thereto.

(SEQ ID NO: 294)
GAPAGPLIVPYNLPLPGGVVPRMLITILGTVKPNANR
IALDFQRGNDVAFHFNPRFNENNRRVIVCNTKLDNNW
GREERQSVFPFESGKPFKIQVLVEPDHFKVAVNDAHL
LQYNHRVKKLNEISKLGISGDIDITSASYTMI.

In some embodiments, the Galectin inhibitor is a peptide, which can bind to, and inhibit Galectin-1 function. In some embodiments, the Galectin inhibitor is the peptide Anginex: Anginex is an anti-angiongenic peptide that binds Galectin-1 (Salomonsson E, et al., (2011) Journal of Biological Chemistry, 286(16):13801-13804). Binding of Anginex to Galectin-1 can interfere with, e.g., the pro-angiongenic effects of Galectin-1.

In some embodiments, the Galectin, e.g., Galectin-1 or Galectin-3, inhibitor is a non-peptidic topomimetic molecule. In some embodiments, the non-peptidic topomimetic Galectin inhibitor is OTX-008 (OncoEthix). In some embodiments, the non-peptidic topomimetic is a non-peptidic topomimetic disclosed in U.S. Pat. No. 8,207,228. OTX-008, also known as PTX-008 or Calixarene 0118, is a selective allosteric inhibitor of Galectin-1. OTX-008 has the chemical name: N-[2-(dimethylamino)ethyl]-2-{[26,27,28-tris({[2-(dimethylamino)ethyl]carbamoyl}methoxy)pentacyclo[19.3.1.1,7.1,.15,]octacosa-1(25),3(28),4,6,9(27),1012,15,17,19(26),21,23-dodecaen-25-yl]oxy}acetamide.

In some embodiments, the Galectin, e.g., Galectin-1 or Galectin-3, inhibitor is a carbohydrate based compound. In some embodiments, the Galectin inhibitor is GR-MD-02 (Galectin Therapeutics). In some embodiments, GR-MD-02 is a Galectin-3 inhibitor. GR-MD-02 is a galactose-pronged polysaccharide also referred to as, e.g., a galactoarabino-rhamnogalaturonate. GR-MD-02 and other galactose-pronged polymers, e.g., galactoarabino-rhamnogalaturonates, are disclosed in U.S. Pat. No. 8,236,780 and U.S. Publication 2014/0086932.

### MEK Inhibitors

In some embodiments, a MEK inhibitor is used in combination with TGFβ inhibitors (and/or PD1, PD-L1, or PD-L2 inhibitor), for treating a disease, e.g., cancer. In some embodiments, the MEK inhibitor is selected from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib.

### Exemplary MEK Inhibitors

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

### Other Exemplary MEK Inhibitors

In some embodiments the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

c-MET Inhibitors

In some embodiments, a c-MET inhibitor is used in combination with TGFβ inhibitors (and/or PD1, PD-L1, or PD-L2 inhibitor), for treating a disease, e.g., cancer. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein.

In some embodiments, the c-MET inhibitor is selected from capmatinib (INC280), JNJ-3887605, AMG 337, LY2801653, MSC2156119J, crizotinib, tivantinib, or golvatinib.

Exemplary c-MET Inhibitors

In some embodiments, the c-MET inhibitor comprises capmatinib (INC280), or a compound described in U.S. Pat. Nos. 7,767,675, 8,461,330.

Other Exemplary c-MET Inhibitors

In some embodiments, the c-MET inhibitor comprises JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways.

In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor comprises AMG 337. AMG 337 is an orally bioavailable inhibitor of c-Met. AMG 337 selectively binds to c-MET, thereby disrupting c-MET signal transduction pathways.

In some embodiments, the c-Met inhibitor comprises LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor comprises MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor comprises crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signalling pathway. Altogether, this agent inhibits tumor cell growth.

In some embodiments, the c-MET inhibitor comprises golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1b), and IL-1alpha (IL-1a). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50).

In some embodiments, an interleukin-1 beta (IL-1β) inhibitor is used in combination with TGFβ inhibitors (and/or PD1, PD-L1, or PD-L2 inhibitor), for treating a disease, e.g., cancer. In some embodiments, the IL-1β inhibitor is selected from canakinumab, gevokizumab, Anakinra, or Rilonacept. In some embodiments, the IL-1β inhibitor is canakinumab.

### Exemplary IL-1β Inhibitors

In some embodiments, the IL-1β inhibitor is canakinumab Canakinumab is also known as ACZ885 or ILARIS® Canakinumab is a human monoclonal IgG1/κ antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of: MEFGLSWVFLVALLRGVQCQVQLVESGGGVVQPGRSLRLSCAASGFTFSVYGMNWVRQAP GKGLEWVAIIWYDGDNQYYADSVKGRFTISRDNSKNTLYLQMNGLRAEDTAVYYCARDLR TGPFDYWGQGTLVTVSS (SEQ ID NO: 297) (disclosed as SEQ ID NO: 1 in U.S. Pat. No. 7,446,175). The light chain variable region of canakinumab has the amino acid sequence of: MLPSQLIGFLLLWVPASRGEIVLTQSPDFQSVTPKEKVTITCRASQSIGSSLHWYQQKPDQSPK LLIKYASQSFSGVPSRFSGSGSGTDFTLTINSLEAEDAAAYYCHQSSSLPFTFGPGTKVDIK (SEQ ID NO: 298) (disclosed as SEQ ID NO: 2 in U.S. Pat. No. 7,446,175).

Canakinumab has been used, e.g., for the treatment of Cryopyrin Associated Periodic Syndromes (CAPS), in adults and children, for the treatment of systemic juvenile idiopathic arthritis (SJIA), for the symptomatic treatment of acute gouty arthritis attacks in adults, and for other IL-1β driven inflammatory diseases. Without wishing to be bound by theory, it is believed that in some embodiments, IL-1β inhibitors, e.g., canakinumab, can increase anti-tumor immune response, e.g., by blocking one or more functions of IL-1b including, e.g., recruitment of immunosuppressive neutrophils to the tumor microenvironment, stimulation of tumor angiogenesis, and/or promotion of metastasis (Dinarello (2010) Eur. J. Immunol. p. 599-606).

In some embodiments, the combination described herein includes an IL-1β inhibitor, canakinumab, or a compound disclosed in WO 2002/16436, and an inhibitor of an immune checkpoint molecule, e.g., an inhibitor of PD-1 (e.g., an anti-PD-1 antibody molecule). IL-1 is a secreted pleotropic cytokine with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50). Canakinumab can bind IL-1b and inhibit IL-1-mediated signalling. Accordingly, in certain embodiments, an IL-1β inhibitor, e.g., canakinumab, enhances, or is used to enhance, an immune-mediated anti-tumor effect of an inhibitor of PD-1 (e.g., an anti-PD-1 antibody molecule).

In some embodiments, the IL-1β inhibitor, canakinumab, or a compound disclosed in WO 2002/16436, and the inhibitor of an immune checkpoint molecule, e.g., an inhibitor of PD-1 (e.g., an anti-PD-1 antibody molecule), each is administered at a dose and/or on a time schedule, that in combination, achieves a desired anti-tumor activity.

### MDM2 Inhibitors

In some embodiments, a mouse double minute 2 homolog (MDM2) inhibitor is used in combination with TGFβ inhibitors (and/or PD1, PD-L1, or PD-L2 inhibitor), for treating a disease, e.g., cancer. The human homolog of MDM2 is also known as HDM2. In some embodiments, an MDM2 inhibitor described herein is also known as a HDM2 inhibitor. In some embodiments, the MDM2 inhibitor is selected from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In an embodiment, an MDM2 inhibitor comprises an inhibitor of p53 and/or a p53/Mdm2 interaction. In an embodiment, the MDM2 inhibitor comprises (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (also known as HDM201), or a compound disclosed in PCT Publication No. WO2013/111105 to treat a disorder, e.g., a disorder described herein. In one embodiment, a therapeutic agent disclosed herein is used in combination with HDM201. In some embodiments, HDM201 is administered orally.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest.

### Pharmaceutical Compositions, Formulations, and Kits

In another aspect, the disclosure provides compositions, e.g., pharmaceutically acceptable compositions, which include a combination described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions described herein can be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. The inhibitors (including antibody inhibitors) described can be in the form of injectable or infusible solutions. The mode of administration is parenteral (e.g., intravenous, subcutaneous, intraperitoneal, intramuscular). In an embodiment, the antibody is administered by intravenous infusion or injection. In another embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (e.g., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A combination or a composition described herein can be formulated into a formulation (e.g., a dose formulation or dosage form) suitable for administration (e.g., intravenous administration) to a subject as described herein. The formulation described herein can be a liquid formulation, a lyophilized formulation, or a reconstituted formulation.

In certain embodiments, the formulation is a liquid formulation. In some embodiments, the formulation (e.g., liquid formulation) comprises a TGFβ inhibitor (e.g., an anti-TGFβ antibody molecule as described herein) and a buffering agent. In some embodiments, the formulation (e.g., liquid formulation) comprises a PD-1 inhibitor (e.g. an anti-PD-1 antibody molecule described herein) and a buffering agent. In some embodiments, the formulation (e.g., liquid formulation) comprises a PD-L1 inhibitor (e.g. an anti-PD-L1 antibody molecule described herein) and a buffering agent. In some embodiments, the formulation (e.g., liquid formulation) comprises a PD-L2 inhibitor (e.g. an anti-PD-L2 antibody) and a buffering agent.

In some embodiments, the formulation (e.g., liquid formulation) comprises an anti-TGF-β or anti-PD1 (or anti-PD-L1/L2) antibody molecule as disclosed herein present at a concentration of about 25 mg/mL to about 250 mg/mL. In some embodiments, the formulation comprises an anti-TGF-β or anti-PD1 (or anti-PD-L1/L2) antibody molecule at a concentration of about 50 mg/mL to about 200 mg/mL. In some embodiments, the formulation comprises an anti-TGF-β or anti-PD1 (or anti-PD-L1/L2) antibody molecule at a concentration of about 60 mg/mL to about 180 mg/mL. In some embodiments, the formulation comprises an anti-TGF-β or anti-PD1 (or anti-PD-L1/L2) antibody molecule at a concentration of about 70 mg/mL to about 150 mg/mL. In some embodiments, the formulation comprises an anti-TGF-β or anti-PD1 (or anti-PD-L1/L2) antibody molecule at a concentration of about 80 mg/mL to about 120 mg/mL. In some embodiments, the formulation comprises an anti-TGF-β or anti-PD1 (or anti-PD-L1/L2) antibody molecule at a concentration of about 90 mg/mL to about 110 mg/mL. In some embodiments, the formulation comprises an anti-TGF-β or anti-PD1 (or anti-PD-L1/L2) antibody molecule at a concentration of about 50 mg/mL to about 150 mg/mL. In some embodiments, the formulation comprises an anti-TGF-β or anti-PD1 (or anti-PD-L1/L2) antibody molecule at a concentration of about 50 mg/mL to about 100 mg/mL. In some embodiments, the formulation comprises an anti-TGF-β or anti-PD1 (or anti-PD-L1/L2) antibody molecule at a concentration of about 150 mg/mL to about 200 mg/mL. In some embodiments, the formulation comprises an anti-TGF-β or anti-PD1 (or anti-PD-L1/L2) antibody molecule at a concentration of about 100 mg/mL to about 200 mg/mL. In some embodiments, the formulation comprises an anti-TGF-β or anti-PD1 (or anti-PD-L1/L2) antibody molecule at a concentration of about 50 mg/mL. In some embodiments, the formulation comprises an anti-TGF-β or anti-PD1 (or anti-PD-L1/L2) antibody molecule at a concentration of about 60 mg/mL. In some embodiments, the formulation comprises an anti-TGF-β or anti-PD1 (or anti-PD-L1/L2) antibody molecule at a concentration of about 70 mg/mL. In some embodiments, the formulation comprises an anti-TGF-β or anti-PD1 (or anti-PD-L1/L2) antibody molecule at a concentration of about 80 mg/mL. In some embodiments, the formulation comprises an anti-TGF-β or anti-PD1 (or anti-PD-L1/L2) antibody molecule at a concentration of about 90 mg/mL. In some embodiments, the formulation comprises an anti-TGF-β or anti-PD1 (or anti-PD-L1/L2) antibody molecule at a concentration of about 100 mg/mL. In some embodiments, the formulation comprises an anti-TGF-β or anti-PD1 (or anti-PD-L1/L2) antibody molecule at a concentration of about 110 mg/mL. In some embodiments, the formulation comprises an anti-TGF-β or anti-PD1 (or anti-PD-L1/L2) antibody molecule at a concentration of about 120 mg/mL. In some embodiments, the formulation comprises an anti-TGF-β or anti-PD1 (or anti-PD-L1/L2) antibody molecule at a concentration of about 130 mg/mL. In some embodiments, the formulation comprises an anti-TGF-β or anti-PD1 (or anti-PD-L1/L2) antibody molecule at a concentration of about 140 mg/mL. In some embodiments, the formulation comprises an anti-TGF-β or anti-PD1 (or anti-PD-L1/L2) antibody molecule at a concentration of about 150 mg/mL. In some embodiments, the formulation comprises an anti-TGF-β or anti-PD1 (or anti-PD-L1/L2) antibody molecule at a concentration of about 80 mg/mL to about 120 mg/mL, e.g., about 100 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of about 1 mM to about 100 mM, e.g., about 2 mM to about 50 mM, about 5 mM to about 40 mM, about 10 mM to about 30 mM, about 15 to about 25 mM, about 5 mM to about 40 mM, about 5 mM to about 30 mM, about 5 mM to about 20 mM, about 5 mM to about 10 mM, about 40 mM to about 50 mM, about 30 mM to about 50 mM, about 20 mM to about 50 mM, about 10 mM to about 50 mM, or about 5 mM to about 50 mM, e.g., about 2 mM, about 5 mM, about 10 mM, about 15 mM, about 20 mM, about 25 mM, about 30 mM, about 35 mM, about 40 mM, about 45 mM, or about 50 mM. In some embodiments, the buffering agent (e.g., histidine buffer) is present at a concentration of about 15 mM to about 25 mM, e.g., about 20 mM. In other embodiments, the buffering agent (e.g., a histidine buffer) has a pH of about 4 to about 7, e.g., about 5 to about 6, e.g., about 5, about 5.5, or about 6. In some embodiments, the buffering agent (e.g., histidine buffer) has a pH of about 5 to about 6, e.g., about 5.5. In certain embodiments, the buffering agent comprises a histidine buffer at a concentration of about 15 mM to about 25 mM (e.g., 20 mM) and has a pH of about 5 to about 6 (e.g., 5.5). In certain embodiments, the buffering agent comprises histidine and histidine-HCl.

In some embodiments, the formulation (e.g., liquid formulation) comprises an antibody molecule as disclosed herein present at a concentration of 80 to 120 mg/mL, e.g., 100 mg/mL; and a buffering agent that comprises a histidine buffer at a concentration of 15 mM to 25 mM (e.g., 20 mM) and has a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of about 50 mM to about 500 mM, e.g., about 100 mM to about 400 mM, about 150 mM to about 300 mM, about 180 mM to about 250 mM, about 200 mM to about 240 mM, about 210 mM to about 230 mM, about 100 mM to about 300 mM, about 100 mM to about 250 mM, about 100 mM to about 200 mM, about 100 mM to about 150 mM, about 300 mM to about 400 mM, about 200 mM to about 400 mM, or about 100 mM to about 400 mM, e.g., about 100 mM, about 150 mM, about 180 mM, about 200 mM, about 220 mM, about 250 mM, about 300 mM, about 350 mM, or about 400 mM. In some embodiments, the formulation comprises a carbohydrate or sucrose present at a concentration of about 200 mM to about 250 mM, e.g., about 220 mM.

In some embodiments, the formulation (e.g., liquid formulation) comprises an antibody molecule as disclosed herein present at a concentration of 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); and a carbohydrate or sucrose present at a concentration of 200 mM to 250 mM, e.g., 220 mM.

In some embodiments, the formulation (e.g., liquid formulation) further comprises a surfactant. In certain embodiments, the surfactant is polysorbate 20. In some embodiments, the surfactant or polysorbate 20) is present at a concentration of about 0.005% to about 0.1% (w/w), e.g., about 0.01% to about 0.08%, about 0.02% to about 0.06%, about 0.03% to about 0.05%, about 0.01% to about 0.06%, about 0.01% to about 0.05%, about 0.01% to about 0.03%, about 0.06% to about 0.08%, about 0.04% to about 0.08%, or about 0.02% to about 0.08% (w/w), e.g., about 0.01%, about 0.02%, about 0.03%, about 0.04%, about 0.05%, about 0.06%, about 0.07%, about 0.08%, about 0.09%, or about 0.1% (w/w). In some embodiments, the formulation comprises a surfactant or polysorbate 20 present at a concentration of about 0.03% to about 0.05%, e.g., about 0.04% (w/w).

In some embodiments, the formulation (e.g., liquid formulation) comprises an antibody molecule as disclosed herein present at a concentration of about 80 to 120 mg/mL, e.g., 100 mg/mL; a buffering agent that comprises a histidine buffer at a concentration of 15 mM to 25 mM (e.g., 20 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., liquid formulation) comprises an antibody molecule as disclosed herein present at a concentration of 100 mg/mL; a buffering agent that comprises a histidine buffer (e.g., histidine/histidine-HCL) at a concentration of 20 mM) and has a pH of 5.5; a carbohydrate or sucrose present at a concentration of 220 mM; and a surfactant or polysorbate 20 present at a concentration of 0.04% (w/w).

In some embodiments, the liquid formulation is prepared by diluting a formulation comprising an antibody molecule described herein. For example, a drug substance formulation can be diluted with a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In certain embodiments, about 0.5 mL to about 10 mL (e.g., about 0.5 mL to about 8 mL, about 1 mL to about 6 mL, or about 2 mL to about 5 mL, e.g., about 1 mL, about 1.2 mL, about 1.5 mL, about 2 mL, about 3 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, or about 10 mL) of the liquid formulation is filled per container (e.g., vial). In other embodiments, the liquid formulation is filled into a container (e.g., vial) such that an extractable volume of at least 1 mL (e.g., at least 1.2 mL, at least 1.5 mL, at least 2 mL, at least 3 mL, at least 4 mL, or at least 5 mL) of the liquid formulation can be withdrawn per container (e.g., vial). In certain embodiments, the liquid formulation is extracted from the container (e.g., vial) without diluting at a clinical site. In certain embodiments, the liquid formulation is diluted from a drug substance formulation and extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any one of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, about 250 mg to about 1500 mg of the antibody molecule as described herein, is present in the container. In some embodiments, the container comprises about 300 mg to about 1250 mg of antibody. In some embodiments, the container comprises about 350 mg to about 1200 mg of antibody. In some embodiments, the container comprises about 400 mg to about 1100 mg of antibody. In some embodiments, the container comprises about 450 mg to about 1000 mg of antibody. In some embodiments, the container comprises about 500 mg to about 900 mg of antibody. In some embodiments, the container comprises about 600 mg to about 800 mg of antibody. In some embodiments, the container comprises about 300 mg of antibody. In some embodiments, the container comprises about 400 mg of antibody. In some embodiments, the container comprises about 500 mg of antibody. In some embodiments, the container comprises about 600 mg of antibody. In some embodiments, the container comprises about 700 mg of antibody. In some embodiments, the container comprises about 800 mg of antibody. In some embodiments, the container comprises about 900 mg of antibody. In some embodiments, the container comprises about 1000 mg of antibody.

In some embodiments, the formulation is a lyophilized formulation. In certain embodiments, the lyophilized formulation is lyophilized or dried from a liquid formulation comprising an antibody molecule described herein. For example, about 1 to about 10 mL, e.g., about 6 to about 8 mL, of a liquid formulation can be filled per container (e.g., vial) and lyophilized.

In some embodiments, the formulation is a reconstituted formulation. In certain embodiments, the reconstituted formulation is reconstituted from a lyophilized formulation comprising an antibody molecule described herein. For example, a reconstituted formulation can be prepared by dissolving a lyophilized formulation in a diluent such that the protein is dispersed in the reconstituted formulation. In some embodiments, the lyophilized formulation is reconstituted with about 1 mL to about 15 mL, e.g., about 5 mL to about 9 mL or about 7 mL, of water or buffer for injection. In certain embodiments, the lyophilized formulation is reconstituted with about 6 mL to about 8 mL of water for injection, e.g., at a clinical site.

In some embodiments, the reconstituted formulation comprises an antibody molecule (e.g., an anti-TGF-β or anti-PD-1 antibody (or anti-PD-L1/2) molecule as disclosed herein) and a buffering agent.

In some embodiments, the reconstituted formulation comprises an comprises an anti-TGF-β or anti-PD1 (or anti-PD-L1/2) antibody molecule at a concentration of about 25 mg/mL to about 250 mg/mL. In some embodiments, the reconstituted formulation comprises an anti-TGF-β or anti-PD1 (or anti-PD-L1/2) antibody molecule at a concentration of about 50 mg/mL to about 200 mg/mL. In some embodiments, the reconstituted formulation comprises an anti-TGF-β or anti-PD1 antibody (or anti-PD-L1/2) molecule at a concentration of about 60 mg/mL to about 180 mg/mL. In some embodiments, the reconstituted formulation comprises an anti-TGF-β or anti-PD1 (or anti-PD-L1/2) antibody molecule at a concentration of about 70 mg/mL to about 150 mg/mL. In some embodiments, the reconstituted formulation comprises an anti-TGF-β or anti-PD1 (or anti-PD-L1/2) antibody molecule at a concentration of about 80 mg/mL to about 120 mg/mL. In some embodiments, the reconstituted formulation comprises an anti-TGF-β or anti-PD1 antibody (or anti-PD-L1/2) molecule at a concentration of about 90 mg/mL to about 110 mg/mL. In some embodiments, the reconstituted formulation comprises an anti-TGF-β or anti-PD1 (or anti-PD-L1/2) antibody molecule at a concentration of about 50 mg/mL to about 150 mg/mL. In some embodiments, the reconstituted formulation comprises an anti-TGF-β or anti-PD1 (or anti-PD-L1/2) antibody molecule at a concentration of about 50 mg/mL to about 100 mg/mL. In some embodiments, the reconstituted formulation comprises an anti-TGF-β or anti-PD1 (or anti-PD-L1/2) antibody molecule at a concentration of about 150 mg/mL to about 200 mg/mL. In some embodiments, the reconstituted formulation comprises an anti-TGF-β or anti-PD1 (or anti-PD-L1/2) antibody molecule at a concentration of about 100 mg/mL to about 200 mg/mL. In some embodiments, the reconstituted formulation comprises an anti-TGF-β or anti-PD1 (or anti-PD-L1/2) antibody molecule at a concentration of about 50 mg/mL. In some embodiments, the reconstituted formulation comprises an anti-TGF-β or anti-PD1 (or anti-PD-L1/2) antibody molecule at a concentration of about 60 mg/mL. In some embodiments, the reconstituted formulation comprises an anti-TGF-β or anti-PD1 (or anti-PD-L1/2) antibody molecule at a concentration of about 70 mg/mL. In some embodiments, the reconstituted formulation comprises an anti-TGF-β or anti-PD1 (or anti-PD-L1/2) antibody molecule at a concentration of about 80 mg/mL. In some embodiments, the reconstituted formulation comprises an anti-TGF-β or anti-PD1 (or anti-PD-L1/2) antibody molecule at a concentration of about 90 mg/mL. In some embodiments, the reconstituted formulation comprises an anti-TGF-β or anti-PD1 (or anti-PD-L1/2) antibody molecule at a concentration of about 100 mg/mL. In some embodiments, the reconstituted formulation comprises an anti-TGF-β or anti-PD1 (or anti-PD-L1/2) antibody molecule at a concentration of about 110 mg/mL. In some embodiments, the reconstituted formulation comprises an anti-TGF-β or anti-PD1 (or anti-PD-L1/2) antibody molecule at a concentration of about 120 mg/mL. In some embodiments, the reconstituted formulation comprises an anti-TGF-β or anti-PD1 (or anti-PD-L1/2) antibody molecule at a concentration of about 130 mg/mL. In some embodiments, the reconstituted formulation comprises an anti-TGF-β or anti-PD1 (or anti-PD-L1/2) antibody molecule at a concentration of about 140 mg/mL. In some embodiments, the reconstituted formulation comprises an anti-TGF-β or anti-PD1 (or anti-PD-L1/2) antibody molecule at a concentration of about 150 mg/mL. In some embodiments, the reconstituted formulation comprises an anti-TGF-β or anti-PD1 (or anti-PD-L1/2) antibody molecule at a concentration of about 80 mg/mL to about 120 mg/mL, e.g., about 100 mg/mL.

In some embodiments, the reconstituted formulation comprises a buffering agent comprising histidine (e.g., a histidine buffer). In certain embodiments, the buffering agent (e.g., histidine buffer) is present at a concentration of about 1 mM to about 100 mM, e.g., about 2 mM to about 50 mM, about 5 mM to about 40 mM, about 10 mM to about 30 mM, about 15 to about 25 mM, about 5 mM to about 40 mM, about 5 mM to about 30 mM, about 5 mM to about 20 mM, about 5 mM to about 10 mM, about 40 mM to about 50 mM, about 30 mM to about 50 mM, about 20 mM to about 50 mM, about 10 mM to about 50 mM, or about 5 mM to about 50 mM, e.g., about 2 mM, about 5 mM, about 10 mM, about 15 mM, about 20 mM, about 25 mM, about 30 mM, about 35 mM, about 40 mM, about 45 mM, or about 50 mM. In some embodiments, the buffering agent (e.g., histidine buffer) is present at a concentration of about 15 mM to about 25 mM, e.g., about 20 mM. In other embodiments, the buffering agent (e.g., a histidine buffer) has a pH of about 4 to about 7, e.g., about 5 to about 6, e.g., about 5, about 5.5, or about 6. In some embodiments, the buffering agent (e.g., histidine buffer) has a pH of about 5 to about 6, e.g., about 5.5. In certain embodiments, the buffering agent comprises a histidine buffer at a concentration of about 15 mM to about 25 mM (e.g., 20 mM) and has a pH of about 5 to about 6 (e.g., 5.5). In certain embodiments, the buffering agent comprises histidine and histidine-HCl.

In some embodiments, the reconstituted formulation comprises an antibody molecule as disclosed herein present at a concentration of about 80 to about 120 mg/mL, e.g., 100 mg/mL; and a buffering agent that comprises a histidine buffer at a concentration of about 15 mM to about 25 mM (e.g., 20 mM) and has a pH of 5 to 6 (e.g., 5.5).

In some embodiments, the reconstituted formulation further comprises a carbohydrate. In certain embodiments, the carbohydrate is sucrose. In some embodiments, the carbohydrate (e.g., sucrose) is present at a concentration of 50 mM to about 500 mM, e.g., about 100 mM to about 400 mM, about 150 mM to about 300 mM, about 180 mM to about 250 mM, about 200 mM to about 240 mM, about 210 mM to about 230 mM, about 100 mM to about 300 mM, about 100 mM to about 250 mM, about 100 mM to about 200 mM, about 100 mM to about 150 mM, about 300 mM to about 400 mM, about 200 mM to about 400 mM, or about 100 mM to about 400 mM, e.g., about 100 mM, about 150 mM, about 180 mM, about 200 mM, about 220 mM, about 250 mM, about 300 mM, about 350 mM, or about 400 mM. In some embodiments, the formulation comprises a carbohydrate or sucrose present at a concentration of about 200 mM to about 250 mM, e.g., about 220 mM.

In some embodiments, the reconstituted formulation comprises an antibody molecule disclosed herein present at a concentration of about 80 to about 120 mg/mL, e.g., 100 mg/mL; a buffering agent that comprises a histidine buffer at a concentration of about 15 mM to about 25 mM (e.g., 20 mM) and has a pH of about 5 to about 6 (e.g., 5.5); and a carbohydrate or sucrose present at a concentration of about 200 mM to about 250 mM, e.g., 220 mM.

In some embodiments, the reconstituted formulation further comprises a surfactant. In certain embodiments, the surfactant is polysorbate 20. In some embodiments, the surfactant or polysorbate 20) is present at a concentration of about 0.005% to about 0.1% (w/w), e.g., about 0.01% to about 0.08%, about 0.02% to about 0.06%, about 0.03% to about 0.05%, about 0.01% to about 0.06%, about 0.01% to about 0.05%, about 0.01% to about 0.03%, about 0.06% to about 0.08%, about 0.04% to about 0.08%, or about 0.02% to about 0.08% (w/w), e.g., about 0.01%, about 0.02%, about 0.03%, about 0.04%, about 0.05%, about 0.06%, about 0.07%, about 0.08%, about 0.09%, or about 0.1% (w/w). In some embodiments, the formulation comprises a surfactant or polysorbate 20 present at a concentration of about 0.03% to about 0.05%, e.g., about 0.04% (w/w).

In some embodiments, the reconstituted formulation comprises an antibody molecule as disclosed herein present at a concentration of about 80 to about 120 mg/mL, e.g., 100 mg/mL; a buffering agent that comprises a histidine buffer at a concentration of about 15 mM to about 25 mM (e.g., 20 mM) and has a pH of about 5 to about 6 (e.g., 5.5); a carbohydrate or sucrose present at a concentration of about 200 mM to about 250 mM, e.g., 220 mM; and a surfactant or polysorbate 20 present at a concentration of about 0.03% to about 0.05%, e.g., 0.04% (w/w).

In some embodiments, the reconstituted formulation comprises an antibody molecule as disclosed herein present at a concentration of 100 mg/mL; a buffering agent that comprises a histidine buffer (e.g., histidine/histidine-HCL) at a concentration of 20 mM) and has a pH of 5.5; a carbohydrate or sucrose present at a concentration of 220 mM; and a surfactant or polysorbate 20 present at a concentration of 0.04% (w/w).

In some embodiments, the formulation is reconstituted such that an extractable volume of at least 1 mL (e.g., at least 1.2 mL, 1.5 mL, 2 mL, 2.5 mL, 3 mL, 3.5 mL, 4 mL, 4.5 mL, 5 mL, 5.5 mL, 6 mL, 6.5 mL, 7 mL, 7.5 mL, 8 mL, 8.5 mL, 9 mL, 9.5 mL or 10 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

In some embodiments, the reconstituted formulation has a fill volume of about 1 mL to about 5 mL. In certain embodiments, the reconstituted formulation has a fill volume of about 2 to about 4 mL. In some embodiments, the reconstituted formulation has a fill volume of about 3 mL. In some embodiments, the reconstituted formulation has a fill volume of about 3.2 mL. In some embodiments, the reconstituted formulation has a fill volume of about 3.4 mL. In some embodiments, the reconstituted formulation has a fill volume of about 3.6 mL. In some embodiments, the reconstituted formulation has a fill volume of about 3.8 mL.

Other exemplary buffering agents that can be used in the formulation described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulation described herein may also contain a tonicity agent, e.g., sodium chloride, and/or a stabilizing agent, e.g., an amino acid (e.g., glycine, arginine, methionine, or a combination thereof).

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 mg/m² to about 310 mg/m², and more typically, about 110 mg/m² to about 130 mg/m². In embodiments, the antibody molecules can be administered by intravenous infusion at a rate of less than 10 mg/min; preferably less than or equal to 5 mg/min to reach a dose of about 1 mg/m² to about 100 mg/m², preferably about 5 mg/m² to about 50 mg/m², about 7 mg/m² to about 25 mg/m² and more preferably, about 10 mg/m². As will be appreciated by the skilled artisan, the route and/or mode of administration will vary depending upon the desired results. In certain embodiments, the active compound can be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The compound (and other ingredients, if desired) may also be enclosed in a hard or soft-shell gelatin capsule, compressed into tablets, or incorporated directly into the subject's diet. For oral therapeutic administration, the compounds can be incorporated with excipients and used in the form of ingestible tablets, buccal tablets, troches, capsules, elixirs, suspensions, syrups, wafers, and the like. To administer a compound of the invention by other than parenteral administration, it can be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The antibody molecule can be administered by intravenous infusion at a rate of more than 20 mg/min, e.g., 20-40 mg/min, and typically greater than or equal to 40 mg/min to reach a dose of about 35 mg/m² to about 440 mg/m², typically about 70 mg/m² to about 310 mg/m², and more typically, about 110 mg/m² to about 130 mg/m². In embodiments, the infusion rate of about 110 mg/m² to about 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 mg/m² to about 100 mg/m², e.g., about 5 mg/m² to about 50 mg/m², about 7 mg/m² to about 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the disclosure is a kit comprising a combination, composition, or formulation described herein. The kit can include one or more other elements including: instructions for use (e.g., in accordance a dosage regimen described herein); other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

### Diagnosing a Subject and Treating Subjects

As used herein, the term “subject” is intended to include human and non-human animals. In some embodiments, the subject is a human subject. The term “non-human animals” includes mammals and non-mammals, such as non-human primates. In some embodiments, the subject is a human. In some embodiments, the subject is a human patient in need of enhancement of an immune response. The combinations described herein are suitable for treating human patients having a disorder that can be treated by modulating (e.g., augmenting or inhibiting) an immune response. In certain embodiments, the patient has or is at risk of having a disorder described herein, e.g., a cancer described herein.

In some cases, the subject that is being treated using the methods disclosed herein, knows that they have a disease or condition, which in some cases would benefit from the methods described here. For example, in some cases, the subject has been tested and/or diagnosed for a disease. This test and/or diagnosis can come from a physician or other qualified medical personnel. In some cases, the test and/or diagnosis can be self-performed based on one or more symptoms, such as bulging masses, lumps, etc. Thus, in some embodiments, the subject may need the methods described herein order to treat their disease or condition. The term “in need thereof” is meant to illustrate that the subject (or the person treating the subject) has knowledge of the existence of a condition or disease (e.g., a proliferative disease such as cancer).

In certain embodiments, the subject has been identified as having TGFβ (1, 2, or 3) expression in their tumor(s) (or tumor microenvironment). In certain embodiments, the subject has been identified as having PD-1 expression in their tumor(s) (or tumor microenvironment). In certain embodiments, the subject has been identified as having PD-L1 expression in their tumor(s) (or tumor microenvironment). In certain embodiments, the subject has been identified as having PD-L2 expression in their tumor(s) (or tumor microenvironment). In some embodiments, the subject has been identified as having both TGFβ (1, 2, or 3) and PD-1 expression in their tumor(s) (or tumor microenvironment). In some embodiments, the subject has been identified as having both TGFβ (1, 2, or 3) and PD-L1 expression in their tumor(s) (or tumor microenvironment). In some embodiments, the subject has been identified as having both TGFβ (1, 2, or 3) and PD-L2 expression in their tumor(s) (or tumor microenvironment). Once these biomarkers are found, then treatment using the methods described can be used.

In some embodiments, the subject is between about 5 kg to about 500 kg. In some embodiments, the subject is between about 10 kg to about 400 kg. In some embodiments, the subject is between about 15 kg to about 300 kg. In some embodiments, the subject is between about 20 kg to about 200 kg. In some embodiments, the subject is between about 25 kg to about 150 kg. In some embodiments, the subject is between about 40 kg to about 125 kg. In some embodiments, the subject is between about 50 kg to about 100 kg. In some embodiments, the subject is between about 65 kg to about 85 kg. In some embodiments, the subject is about 40 kg. In some embodiments, the subject is about 45 kg. In some embodiments, the subject is about 50 kg. In some embodiments, the subject is about 55 kg. In some embodiments, the subject is about 60 kg. In some embodiments, the subject is about 65 kg. In some embodiments, the subject is about 70 kg. In some embodiments, the subject is about 75 kg. In some embodiments, the subject is about 80 kg. In some embodiments, the subject is about 85 kg. In some embodiments, the subject is about 90 kg. In some embodiments, the subject is about 95 kg. In some embodiments, the subject is about 100 kg. In some embodiments, the subject is about 110 kg. In some embodiments, the subject is about 120 kg. In some embodiments, the subject is about 130 kg. In some embodiments, the subject is about 140 kg. In some embodiments, the subject is about 150 kg.

### CancerXXXX

In some embodiments, the methods are used to treat a cancer such as myelofibrosis (e.g., primary myelofibrosis (PMF), post-essential thrombocythemia myelofibrosis (PET-MF), post-polycythemia vera myelofibrosis (PPV-MF)), leukemia (e.g., an acute myeloid leukemia (AML), e.g., a relapsed or refractory AML or a de novo AML; or a chronic lymphocytic leukemia (CLL)), a lymphoma (e.g., T-cell lymphoma, B-cell lymphoma, a non-Hodgkin's lymphoma, or a small lymphocytic lymphoma (SLL)), a myeloma (e.g., multiple myeloma), a lung cancer (e.g., a non-small cell lung cancer (NSCLC) (e.g., a NSCLC with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS) (e.g., low risk MDS), a prostate cancer, a breast cancer (e.g., a breast cancer that does not express one, two or all of estrogen receptor, progesterone receptor, or Her2/neu, e.g., a triple negative breast cancer), a colorectal cancer, a nasopharyngeal cancer, a duodenal cancer, an endometrial cancer, a pancreatic cancer (e.g., pancreatic ductal adenocarcinoma (PDAC)), a head and neck cancer (e.g., head and neck squamous cell carcinoma (HNSCC), an anal cancer, a gastro-esophageal cancer, a thyroid cancer (e.g., anaplastic thyroid carcinoma), a cervical cancer, or a neuroendocrine tumor (NET) (e.g., an atypical pulmonary carcinoid tumor).

In certain embodiments, the patient is not suitable for a standard therapeutic regimen with established benefit in patients with one or more of the cancers described herein. In some embodiments, the subject is unfit for a chemotherapy. In some embodiments, the chemotherapy is an intensive induction chemotherapy. For example, the methods described herein can be used for the treatment of adult patients with one or more of the cancers as described. In certain embodiments, the inhibitors (TGFβ and/or PD1)) are administered in an amount effective to treat a cancer or a symptom thereof.

The compositions, formulations, or methods described herein can be used to inhibit the growth of cancerous tumors. Alternatively, the compositions, formulations, or methods described herein can be used in combination with one or more of: a standard of care treatment for cancer, another antibody or antigen-binding fragment thereof, an immunomodulator (e.g., an activator of a costimulatory molecule or an inhibitor of an inhibitory molecule); a vaccine, e.g., a therapeutic cancer vaccine; or other forms of cellular immunotherapy, as described herein. In one embodiment, the methods are suitable for the treatment of cancer in vivo.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes performing the methods described herein, or a composition or formulation described herein, in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, hematological cancers, solid tumors, soft tissue tumors, and metastatic lesions.

Examples of solid tumors include, but are not limited to, malignancies, e.g., sarcomas, and carcinomas (including adenocarcinomas and squamous cell carcinomas), of the various organ systems, such as those affecting liver, lung, breast, lymphoid, gastrointestinal (e.g., colon), anal, genitals and genitourinary tract (e.g., renal, urothelial, bladder), prostate, CNS (e.g., brain, neural or glial cells), head and neck, skin, pancreas, and pharynx. Adenocarcinomas include malignancies such as most colon cancers, rectal cancer, renal cancer (e.g., renal-cell carcinoma (e.g., clear cell or non-clear cell renal cell carcinoma), liver cancer, lung cancer (e.g., non-small cell carcinoma of the lung (e.g., squamous or non-squamous non-small cell lung cancer)), cancer of the small intestine, and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. The cancer can be at an early, intermediate, late stage or metastatic cancer. Metastatic lesions of the aforementioned cancers can also be treated or prevented using the combinations described herein.

In certain embodiments, the cancer is a solid tumor. In some embodiments, the cancer is an ovarian cancer. In other embodiments, the cancer is a lung cancer, e.g., a small cell lung cancer (SCLC) or a non-small cell lung cancer (NSCLC). In other embodiments, the cancer is a mesothelioma. In other embodiments, the cancer is a skin cancer, e.g., a Merkel cell carcinoma or a melanoma. In other embodiments, the cancer is a kidney cancer, e.g., a renal cell carcinoma (RCC). In other embodiments, the cancer is a bladder cancer. In other embodiments, the cancer is a soft tissue sarcoma, e.g., a hemangiopericytoma (HPC). In other embodiments, the cancer is a bone cancer, e.g., a bone sarcoma. In other embodiments, the cancer is a colorectal cancer. In other embodiments, the cancer is a pancreatic cancer (e.g., PDAC). In other embodiments, the cancer is a nasopharyngeal cancer. In other embodiments, the cancer is a breast cancer. In other embodiments, the cancer is a duodenal cancer. In other embodiments, the cancer is an endometrial cancer. In other embodiments, the cancer is an adenocarcinoma, e.g., an unknown adenocarcinoma. In other embodiments, the cancer is a liver cancer, e.g., a hepatocellular carcinoma. In other embodiments, the cancer is a cholangiocarcinoma. In other embodiments, the cancer is a sarcoma. In certain embodiments, the cancer is a myelodysplastic syndrome (MDS) (e.g., a high risk MDS or low risk MDS).

In another embodiment, the cancer is a carcinoma (e.g., advanced or metastatic carcinoma), melanoma or a lung carcinoma, e.g., a non-small cell lung carcinoma. In one embodiment, the cancer is a lung cancer, e.g., a non-small cell lung cancer or small cell lung cancer. In some embodiments, the non-small cell lung cancer is a stage I (e.g., stage Ia or Ib), stage II (e.g., stage IIa or IIb), stage III (e.g., stage IIIa or IIIb), or stage IV, non-small cell lung cancer. In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In another embodiment, the cancer is a hepatocarcinoma, e.g., an advanced hepatocarcinoma, with or without a viral infection, e.g., a chronic viral hepatitis. In another embodiment, the cancer is a prostate cancer, e.g., an advanced prostate cancer. In yet another embodiment, the cancer is a myeloma, e.g., multiple myeloma. In yet another embodiment, the cancer is a renal cancer, e.g., a renal cell carcinoma (RCC) (e.g., a metastatic RCC, a non-clear cell renal cell carcinoma (nccRCC), or clear cell renal cell carcinoma (CCRCC)).

In some embodiments, the cancer is an MSI-high cancer. In some embodiments, the cancer is a metastatic cancer. In other embodiments, the cancer is an advanced cancer. In other embodiments, the cancer is a relapsed or refractory cancer.

Exemplary cancers whose growth can be inhibited using the methods, compositions, or formulations, as disclosed herein, include cancers typically responsive to immunotherapy. Additionally, refractory or recurrent malignancies can be treated using the combinations described herein.

Examples of other cancers that can be treated include, but are not limited to, basal cell carcinoma, biliary tract cancer; bladder cancer; bone cancer; brain and CNS cancer; primary CNS lymphoma; neoplasm of the central nervous system (CNS); breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia (including acute myeloid leukemia, chronic myeloid leukemia, acute lymphoblastic leukemia, chronic lymphocytic leukemia, chronic or acute leukemia); liver cancer; lung cancer (e.g., small cell and non-small cell); lymphoma including Hodgkin's and non-Hodgkin's lymphoma; lymphocytic lymphoma; melanoma, e.g., cutaneous or intraocular malignant melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, hepatocarcinoma, cancer of the anal region, carcinoma of the fallopian tubes, carcinoma of the vagina, carcinoma of the vulva, cancer of the small intestine, cancer of the endocrine system, cancer of the parathyroid gland, cancer of the adrenal gland, sarcoma of soft tissue, cancer of the urethra, cancer of the penis, solid tumors of childhood, spinal axis tumor, brain stem glioma, pituitary adenoma, Kaposi's sarcoma, epidermoid cancer, squamous cell cancer, T-cell lymphoma, environmentally induced cancers including those induced by asbestos, as well as other carcinomas and sarcomas, and combinations of said cancers.

The methods and therapies described herein can include a composition co-formulated with, and/or co-administered with, one or more therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

When administered in combination, the TGF-β inhibitor, the PD-1 inhibitor, PD-L1 inhibitor, or PD-L2 inhibitor, one or more additional agents, or all, can be administered in an amount or dose that is higher, lower or the same than the amount or dosage of each agent used individually, e.g., as a monotherapy. In certain embodiments, the administered amount or dosage of the TGF-β inhibitor, PD-1 inhibitor, PD-L1 inhibitor, or PD-L2 inhibitor, one or more additional agents, or all, is lower (e.g., at least 20%, at least 30%, at least 40%, or at least 50%) than the amount or dosage of each agent used individually, e.g., as a monotherapy. In other embodiments, the amount or dosage of the TGF-β inhibitor, PD-1 inhibitor, PD-L1 inhibitor, or PD-L2 inhibitor, one or more additional agents, or all, that results in a desired effect (e.g., treatment of cancer) is lower (e.g., at least 20%, at least 30%, at least 40%, or at least 50% lower).

In other embodiments, the additional therapeutic agent is from the agents listed in Table 6 of WO 2017/019897. In some embodiments, the additional therapeutic agent is one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11(3-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase, e.g., as described in Table 6 of WO 2017/019897.

### EXAMPLES

### Example 1: Drug Product

The TGFβ inhibitor known as NIS793, was made into a powder which can be used as a solution for infusion. The powder was provided in glass vials with rubber stoppers which were sealed with a flip-off caps. Each vial contained 100 mg of NIS793 lyophilisate. The drug product was manufactured using a standard aseptic process. In addition to NIS793, the drug product contained the following pharmaceutical excipients: L-histidine/L-histidine hydrochloride monohydrate, polysorbate 20 and sucrose. The vial was provided with a 20% overfill to allow withdrawal of the entire dose.

The drug product was designed to be reconstituted with 1 mL of sterile water for injection prior to administration resulting in a 100 mg/mL NIS793 solution.

NIS793 concentrate for solution for infusion was provided in glass vials with rubber stoppers which were sealed with a flip-off caps. Each vial contained 700 mg of NIS793 in 7 mL of solution. The drug product solution contained the same quantitative and qualitative excipients as NIS793 powder for solution for infusion after reconstitution in sterile water. Similarly, a 7% overfill was provided to allow withdrawal of the entire dose.

### Example 2: Human Studies

Drug product containing NIS793 as described in Example 1 were used in clinical trials. A summary of ongoing human trials are provided in Table 5 below.

TABLE 0
Ongoing human study

| | | | | |
|---|---|---|---|---|
| | Population | No. of | | |
| | (No. of | Subjects | | |
| | enrolled | exposed | | NIS793 Dose/Frequency/ |
| Study | subjects) | to NIS793 | Study Title | Formulation |

Patient studies

| | | | | |
|---|---|---|---|---|
| CNIS793X2101 | Advanced | 120 | A phase I/lb, open-label, | 0.3-30 mg/kg Q3W |
| | solid tumors | | multi-center dose | 20-30 mg/kg Q2W |
| | (120) | | escalation study of NIS793 | 2100 mg Q3W |
| | | | in combination with | as single agent or in combination with |
| | | | PDR001 in adult patients | PDR001 |
| | | | with advanced | concentrate for solution for infusion |
| | | | malignancies | (liquid in vial; 25 mg NIS793 in 0.25 |
| | | | | mL). This drug product has been |
| | | | | replaced by powder for solution for |
| | | | | infusion after treatment of the initial 2 |
| | | | | cohorts. |
| | | | | powder for solution for infusion |
| | | | | (lyophilisate in vial; 100 mg NIS793) |

One human study has been initiated and is ongoing: The first-in-human study, CNIS793X2101, “A phase I/Ib, open-label, multi-center dose escalation study of NIS793 in combination with PDR001 in adult patients with advanced malignancies”. A total of 120 patients were treated with NIS793 as single agent or in combination with PDR001 (Table 5).

### Pharmacokinetics, Metabolism and Pharmacodynamics in Humans

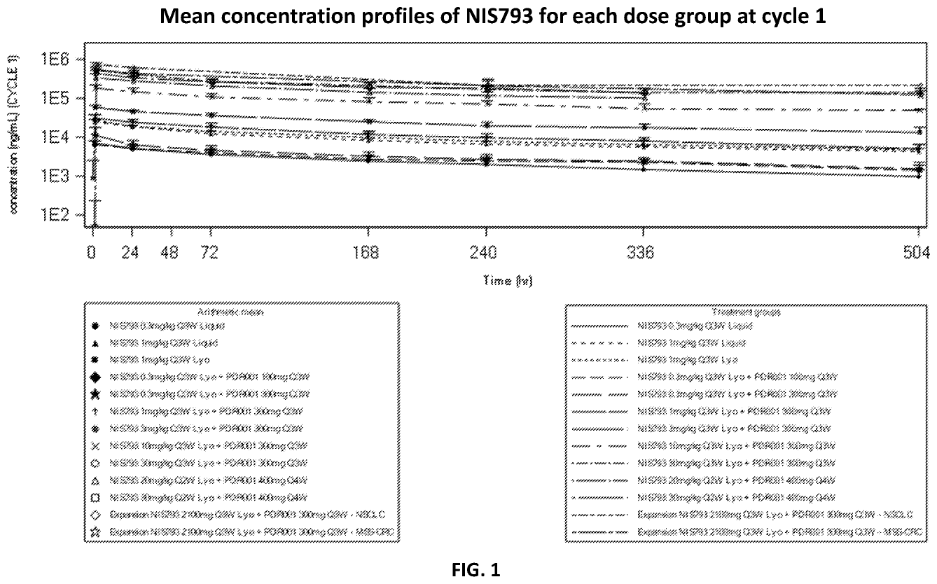

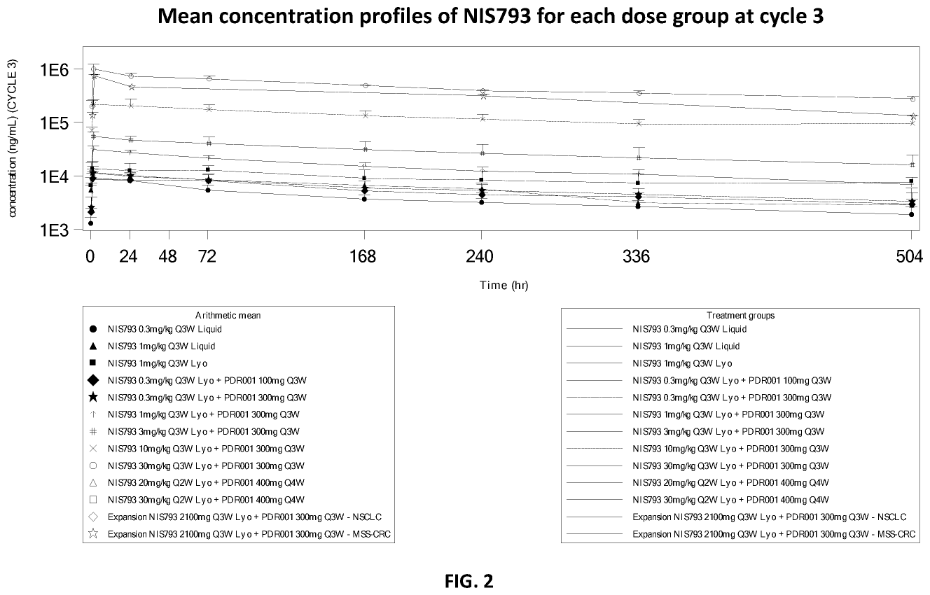

The PK data of NIS793 from the CNIS793X2101 study (75 patients, cut off date of 4 May 2020) was characterized. A summary of derived PK parameters estimates for NIS793 as single agent (NIS793: 0.3-1 mg/kg Q3W) and in combination with PDR001 (NIS793/PDR001: 0.3 mg/kg/100 mg Q3W, 0.3-30 mg/kg/300 mg Q3W and 20-30 mg/kg Q2W/400 mg Q4W) in escalation cohorts is presented in Table 6 for cycle 1 and Table 7 for cycle 3. In addition, a summary of derived PK parameters estimates for NIS793 in a combination with PDR001 (NIS793/PDR001: 2100 mg/300 mg Q3W) in expansion cohort in MSS-CRC and NSCLC is presented in Table 8. Mean concentration-time profiles for each dose cohort of NIS793 are plotted in FIG. 1 for cycle 1 and FIG. 2 for cycle 3.

Following administration of NIS793 via a 30 minute intravenous infusion, approximately dose-proportional increase in NIS793 exposure (i.e. Cycle 1 Cmax and AUClast) was observed from 0.3 mg/kg to 30 mg/kg. Moderate accumulation (approximately up to 2.0-fold) of NIS793 was observed based on ratio of AUClast and Cmax on cycle 3 versus cycle 1. PK variability was low to moderate as illustrated by between subject variability (CV %) (e.g. 12.1 to 73.3% for Cmax).

PDR001 was administered in combination with NIS793 at three doses and two dosing regimens (100 or 300 mg Q3W and 400 mg Q4W). The PK of PDR001 in combination with NIS793 appears to be similar to the single agent data from the PDR001 clinical trial studies.

TABLE 6
Summary of PK parameters for NIS793 by treatment on cycle 1 in single agent and combination studies (escalation)

| | | Cycle1 | | | | | |
|---|---|---|---|---|---|---|---|
| Treatment | Statistics | AUClast (h*ng/mL) | | Clast (ng/mL) | | Cmax (ng/mL) | |
| NIS793 0.3 mg/kg Q3W Liquid (N = 3) | N | 3 | | 3 | | 3 | |
| | Mean (SD) | 1140000 | (439000) | 1060 | (358) | 7180 | (923) |
| | CV % mean | 38.7 | | 33.7 | | 12.9 | |
| NIS793 1 mg/kg Q3W Liquid (N = 4) | N | 4 | | 4 | | 4 | |
| | Mean (SD) | 4170000 | (478000) | 4320 | (814) | 24900 | (3580) |
| | CV % mean | 11.5 | | 18.9 | | 14.4 | |
| NIS793 1 mg/kg Q3W Lyophilisate (N = 4) | N | 4 | | 4 | | 4 | |
| | Mean (SD) | 4770000 | (1460000) | 4780 | (1640) | 27600.0 | (10400) |
| | CV % mean | 30.6 | | 34.4 | | 37.8 | |
| NIS793 0.3 mg/kg Q3W + PDR 100 mg Q3W | N | 5 | | 5 | | 5 | |
| (N = 4) | Mean (SD) | 1360000 | (339000) | 1400 | (498) | 6440 | (1560) |
| | CV % mean | 24.9 | | 39.7 | | 24.2 | |
| NIS793 0.3 mg/kg Q3W + PDR 300 mg Q3W | N | 5 | | 5 | | 5 | |
| (N = 5) | Mean (SD) | 1700000 | (539000) | 1530 | (722) | 11200 | (6530) |
| | CV % mean | 31.6 | | 47.0 | | 58.3 | |
| NIS793 1 mg/kg Q3W + PDR 300 mg Q3W | N | 5 | | 5 | | 5 | |
| (N = 5) | Mean (SD) | 5850000 | (1510000) | 8790 | (8440) | 30800 | (6040) |
| | CV % mean | 25.7 | | 96.1 | | 19.6 | |
| NIS793 3 mg/kg Q3W + PDR 300 mg Q3W | N | 5 | | 5 | | 5 | |
| (N = 5) | Mean (SD) | 11400000 | (2330000) | 14300 | (4830) | 58400 | (7750) |
| | CV % mean | 20.4 | | 33.8 | | 13.3 | |
| NIS793 10 mg/kg Q3W + PDR 300 mg Q3W | N | 5 | | 5 | | 5 | |
| (N = 5) | Mean (SD) | 3750000 | (10200000) | 44800 | (9630) | 181000 | (41500) |
| | CV % mean | 27.2 | | 21.5 | | 22.9 | |
| NIS793 30 mg/kg Q3W + PDR 300 mg Q3W | N | 6 | | 6 | | 6 | |
| (N = 7) | Mean (SD) | 90900000 | (25200000) | 133000 | (336900) | 519000 | (100000) |
| | CV % mean | 27.1 | | 27.7 | | 19.3 | |
| NIS793 20 mg/kg Q2W + PDR 400 mg Q4W | N | 6 | | 6 | | 6 | |
| (N = 11) | Mean (SD) | 54500000 | (15600000) | 107000 | (29400) | 333000 | (85600) |
| | CV % mean | 28.7 | | 27.5 | | 25.7 | |
| NIS793 30 mg/kg Q2W + PDR 400 mg Q4W | N | 4 | | 4 | | 4 | |
| (N = 11) | Mean (SD) | 69100000 | (18100000) | 154000 | (37700) | 439000 | (96900) |

| | | | |
|---|---|---|---|
| CV % mean | 26.2 | 24.5 | 23.4 |

N: number of patients with preliminary PK; n: number of patients with available PK parameter values; “—”: Not applicable; CV %: coefficient of variation (%) = sd/mean*100.

TABLE 7
Summary of PK parameters for NIS793 by treatment on cycle 3 in single agent and combination studies (escalation)

| | | Cycle3 | | | | | |
|---|---|---|---|---|---|---|---|
| Treatment | Statistics | AUClast (h*ng/mL) | | Clast (ng/mL) | | Cmax (ng/mL) | |
| NIS793 0.3 mg/kg Q3W Liquid (N = 3) | N | 2 | | 2 | | 2 | |
| | Mean (SD) | 1820000 | (467000) | 1900 | (735) | 8750 | (2340) |
| | CV % | 25.7 | | 38.7 | | 26.8 | |
| | mean | | | | | | |
| NIS793 1 mg/kg Q3W Liquid (N = 4) | N | 3 | | 3 | | 3 | |
| | Mean (SD) | 2910000 | (877000) | 4290 | (2480) | 9830 | (3500) |
| | CV % | 30.2 | | 57.8 | | 35.6 | |
| | mean | | | | | | |
| NIS793 1 mg/kg Q3W Lyophilisate (N = 4) | N | 3 | | 3 | | 3 | |
| | Mean (SD) | 4090000 | (2200000) | 6350 | (2860) | 13800 | (4970) |
| | CV % | 54.0 | | 45.1 | | 36.1 | |
| | mean | | | | | | |
| NIS793 0.3 mg/kg Q3W + PDR 100 mg Q3W | N | 2 | | 2 | | 2 | |
| (N = 4) | Mean (SD) | 2530000 | (441000) | 2960 | (750) | 8990 | (2990) |
| | CV % | 17.4 | | 25.3 | | 33.3 | |
| | mean | | | | | | |
| NIS793 0.3 mg/kg Q3W + PDR 300 mg Q3W | N | 4 | | 4 | | 4 | |
| (N = 5) | Mean (SD) | 2960000 | (753000) | 3330 | (1540) | 11600 | (3530) |
| | CV % | 25.4 | | 46.2 | | 30.3 | |
| | mean | | | | | | |
| NIS793 1 mg/kg Q3W + PDR 300 mg Q3W | N | 3 | | 3 | | 3 | |
| (N = 5) | Mean (SD) | 5630000 | (1500000) | 9710 | (2780) | 31300 | (5070) |
| | CV % | 26.6 | | 28.6 | | 16.2 | |
| | mean | | | | | | |
| NIS793 3 mg/kg Q3W + PDR 300 mg Q3W | N | 3 | | 3 | | 3 | |
| (N = 5) | Mean (SD) | 13900000 | (5910000) | 16100 | (8810) | 55200 | (11300) |
| | CV % | 42.5 | | 54.8 | | 20.5 | |
| | mean | | | | | | |
| NIS793 10 mg/kg Q3W + PDR 300 mg Q3W | N | 4 | | 4 | | 4 | |
| (N = 5) | Mean (SD) | 69200000 | (9110000) | 95900 | (36800) | 220000 | (48600) |
| | CV % | 13.2 | | 38.4 | | 22.1 | |
| | mean | | | | | | |
| NIS793 30 mg/kg Q3W + PDR 300 mg Q3W | N | 2 | | 3 | | 3 | |
| (N = 7) | Mean (SD) | 242000000 | (24000000) | 228000 | (80800) | 713000 | (522000) |
| | CV % | 9.93 | | 35.5 | | 73.3 | |
| | mean | | | | | | |
| NIS793 20 mg/kg Q2W + PDR 400 mg Q4W | N | 3 | | 3 | | 3 | |
| (N = 6) | Mean (SD) | 149000000 | (138000000) | 154000 | (45200) | 524000 | (155000) |

| | CV % | 92.7 | 29.3 | 29.6 |
|---|---|---|---|---|
| | mean | | | |
| NIS793 30 mg/kg Q2W + PDR 400 mg Q4W | N | 0 | 0 | 0 |
| (N = 11) | Mean (SD) | — | — | — |
| | CV % | — | — | — |
| | mean | | | |

N: number of patients with preliminary PK; n: number of patients with available PK parameter values; “—”: Not applicable; CV %: coefficient of variation (%) = sd/mean*100.

TABLE 8
Summary of PK parameters for NIS793 2100 mg Q3W on cycles 1 and 3 in combination studies (expansion)

| | | Cycle1 | | | | | |
|---|---|---|---|---|---|---|---|
| Treatment | Statistics | AUClast (h*ng/mL) | | Clast (ng/mL) | | Cmax (ng/mL) | |
| NIS793 2100 mg Q3W + PDR 300 | N | 21 | | 21 | | 21 | |
| mg Q3W, MSS-CRC (N = 40) | Mean (SD) | 94700000 | (51500000) | 206000 | (150000) | 547000 | (121000) |
| | CV % mean | 54.4 | | 72.7 | | 22.2 | |
| NIS793 2100 mg Q3W + PDR 300 | N | 2 | | 2 | | 2 | |
| mg Q3W, NSCLC (N = 20) | | | | | | | |
| | Mean (SD) | 140000000 | (59900000) | 180000 | (50200) | 743000 | (79200) |

| | | | |
|---|---|---|---|
| CV % mean | 42.9 | 28.0 | 10.7 |

| | | Cycle3 | | |
|---|---|---|---|---|
| Treatment | Statistics | AUClast (h*ng/mL) | Clast (ng/mL) | Cmax (ng/mL) |
| NIS793 2100 mg Q3W + PDR 300 | N | 3 | 3 | 3 |
| mg Q3W, MSS-CRC (N = 40) | Mean (SD) | 95500000 (88500000) | 341000 (313000) | 590000 (319000) |
| | CV % mean | 92.6 | 91.7 | 54.0 |

N: number of patients with preliminary PK; n: number of patients with available PK parameter values; “—”: Not applicable; CV %: coefficient of variation (%) = sd/mean*100.

Population PK analysis on the concentration data from the dose escalation phase of the study CNIS793X2101 was used to describe the pharmacokinetic characteristics of NIS793 including the impact of weight as a covariate on clearance and volume of distribution. The analysis suggested that the pharmacokinetics of NIS793 can be well described using a two compartment model with first order elimination from the central compartment. This is consistent with the observation that NIS793 PK appears dose proportional and time-independent based on the non-compartmental analyses.

Although body weight (BW) is a covariate on clearance in the population PK model with the estimated exponent of 0.55 (CV %=40%) from the power model, the predicted exposure and trough concentration at steady state between weight-based and fixed dosing regimens were comparable across different BW categories. This analysis supports the use of fixed or flat dosing on a mg basis irrespective of patient body weight as weight-based dosing does not decrease inter-individual variability. Model-based simulations indicated that a dose of 2100 mg would match exposure observed at 30 mg/kg. Further a dose of 1400 mg would also match exposure observed at 20 mg/kg.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A method of treating a proliferative disease in a subject in need thereof comprising administering to the subject a TGF-β antibody at a dose of about 16 mg/kg to about 50 mg/kg, once every two or three weeks, wherein the TGF-β antibody comprises a heavy chain CDR1, CDR2, and CDR3, of SEQ ID NOs: 1, 2, and 3, respectfully, and a light chain CDR1, CDR2, and CDR3, of SEQ ID NOs: 4, 5, and 6, respectfully.

2. The method of claim 1, wherein the TGF-β antibody is administered at a dose of about 20 mg/kg.

3. The method of claim 1 or 2, wherein the TGF-β antibody is administered at a dose of about 30 mg/kg.

4. A method of treating a proliferative disease in a subject in need thereof comprising administering to the subject a TGF-β antibody at a dose of about 1200 mg to about 1600 mg, once every two or three weeks, wherein the TGF-β antibody comprises a heavy chain CDR1, CDR2, and CDR3, of SEQ ID NOs: 1, 2, and 3, respectfully, and a light chain CDR1, CDR2, and CDR3, of SEQ ID NOs: 4, 5, and 6, respectfully.

5. The method of claim 4, wherein the TGF-β antibody is administered at a dose of about 1400 mg.

6. The method of claim 4 or 5, wherein the TGF-β antibody is administered every two weeks.

7. A method of treating a proliferative disease comprising administering to a subject in need thereof a TGF-β antibody at a dose of about 1900 mg to about 2300 mg, once every two or three weeks, wherein the TGF-β antibody comprises a heavy chain CDR1, CDR2, and CDR3, of SEQ ID NOs: 1, 2, and 3, respectfully, and a light chain CDR1, CDR2, and CDR3, of SEQ ID NOs: 4, 5, and 6, respectfully.

8. The method of claim 7, wherein the TGF-β antibody is administered at a dose of about 2100 mg.

9. The method of claim 7 or 8, wherein the TGF-β antibody is administered every three weeks.

10. The method of claim 7 or 8, wherein the TGF-β antibody is administered every two weeks.

11. The method of any one of claims 1 to 10, wherein the TGF-β antibody comprises the heavy chain variable region and the light chain variable region set out in amino acid sequence SEQ ID NOs: 7 and 8, respectively.

12. The method of any one of claims 1 to 11, wherein the TGF-β antibody comprises the heavy chain and light chain set out in amino acid sequence of SEQ ID NOs: 9 and 10, respectively.

13. The method of any one of claims 1 to 12, wherein the TGF-β antibody is a monoclonal antibody.

14. The method of any one of claims 1 to 13, wherein the antibody is a multispecific antibody.

15. The method of claim 14, wherein the multispecific antibody is a bispecific antibody.

16. The method of claim 14, wherein the multispecific antibody is a trispecific antibody.

17. The method of any one of claims 1 to 16, wherein the TGF-β inhibitor is administered over a period of about 20 to about 40 minutes.

18. The method of claim 17, wherein the TGF-β inhibitor is administered over a period of about 30 minutes.

19. The method of any one of claims 1 to 18, wherein the subject is about 70 kg.

20. The method of any one of claims 1 to 19, further comprising administering to the subject a checkpoint inhibitor.

21. The method of claim 20, wherein the checkpoint inhibitor is a PD1 inhibitor.

22. The method of claim 21, wherein the PD1 inhibitor is an anti-PD1 antibody.

23. The method of claim 22, wherein the anti-PD1 antibody is spartalizumab, nivolumab, pembrolizumab, pidilizumab, MEDI0680, REGN2810, TSR-042, PF-06801591, BGB-A317, BGB-108, INCSHR1210, AMP-224, or any combination thereof.

24. The method of claim 22 or 23, wherein the anti-PD1 antibody is spartalizumab.

25. The method of claim 24, wherein spartalizumab is administered at a flat dose.

26. The method of claim 25, wherein spartalizumab is administered at a dose of about 300 mg or about 400 mg, once every three weeks or once every four weeks.

27. The method of any one of claims 23 to 26, wherein spartalizumab is administered at a dose of about 300 mg spartalizumab, once every three weeks.

28. The method of any one of claims 23 to 26, wherein spartalizumab is administered at a dose of about 400 mg spartalizumab, once every four weeks.

29. The method of any one of claims 1 to 28, wherein the TGF-β inhibitor and/or PD1 inhibitor is administered intravenously.

30. The method of any one of claims 20 to 29, wherein the TGF-β inhibitor is administered on the same day as the checkpoint inhibitor.

31. The method of any one of claims 20 to 29, wherein the TGF-β inhibitor is administered before the checkpoint inhibitor is administered.

32. The method of any one of claims 20 to 29, wherein the TGF-β inhibitor is administered after the checkpoint inhibitor is administered.

33. The method of any one of claims 20 to 29, wherein the TGF-β inhibitor is administered at the same time as the checkpoint inhibitor.

34. The method of any one of claims 1 to 33, wherein the proliferative disease is a cancer.

35. The method of claim 34, wherein the cancer is a myelofibrosis, a leukemia, a lymphoma, a myeloma, a lung cancer, a skin cancer, an ovarian cancer, a mesothelioma, a gastrointestinal cancer, a bladder cancer, a soft tissue sarcoma, a bone cancer, a kidney cancer, a liver cancer, a cholangiocarcinoma, a sarcoma, a myelodysplastic syndrome (e.g., low risk or high risk myelodysplastic syndrome), a prostate cancer, a breast cancer (e.g., triple negative breast cancer), a colorectal cancer, a nasopharyngeal cancer, a duodenal cancer, an endometrial cancer, a pancreatic cancer, a head and neck cancer, an anal cancer, a gastro-esophageal cancer, a thyroid cancer, a cervical cancer, or a neuroendocrine tumor.

36. The method of claim 35, wherein the cancer is a pancreatic cancer.

37. The method of claim 36, wherein the pancreatic cancer is pancreatic ductal adenocarcinoma (PDAC).

38. The method of claim 35, wherein the cancer is a gastrointestinal cancer.

39. The method of claim 38, wherein the gastrointestinal cancer is colorectal cancer.

40. The method of claim 35, wherein the cancer is myelofibrosis.

41. The method of claim 35, wherein the cancer is myelodysplastic syndrome.

42. The method of claim 35, wherein the cancer is breast cancer.

43. The method of claim 42, wherein the breast cancer is triple negative breast cancer.

44. The method of any one of claims 1 to 43, wherein the TGF-β inhibitor and/or checkpoint inhibitor is given until remission.

45. The method of any one of claims 1 to 44, further comprising administering to the subject one or more anticancer therapies.

46. The method of claim 45, wherein the anticancer therapy is a standard of care therapy or an anticancer therapeutic.

47. A method of treating pancreatic ductal adenocarcinoma comprising administering to a subject in need thereof a TGF-β antibody at a dose of 2100 mg, once every two or three weeks, wherein the TGF-β antibody comprises the heavy chain and light chain set out in amino acid sequence of SEQ ID NOs: 9 and 10, respectively.

48. A method of treating colorectal cancer comprising administering to a subject in need thereof a TGF-β antibody at a dose of 2100 mg, once every two or three weeks, wherein the TGF-β antibody comprises the heavy chain and light chain set out in amino acid sequence of SEQ ID NOs: 9 and 10, respectively.

49. A method of treating pancreatic ductal adenocarcinoma comprising administering to a subject in need thereof a TGF-β antibody at a dose of 1400 mg, once every two weeks, wherein the TGF-β antibody comprises the heavy chain and light chain set out in amino acid sequence of SEQ ID NOs: 9 and 10, respectively.

50. A method of treating colorectal cancer comprising administering to a subject in need thereof a TGF-β antibody at a dose of 1400 mg, once every three weeks, wherein the TGF-β antibody comprises the heavy chain and light chain set out in amino acid sequence of SEQ ID NOs: 9 and 10, respectively.

51. The method of any one of claims 47 to 50, further comprising administering spartalizumab at a dose of about 300 mg once every three weeks.

52. The method of any one of claims 47 to 50, further comprising administering spartalizumab at a dose of about 400 mg once every four weeks.

53. A pharmaceutical composition comprising a TGF-β antibody and a pharmaceutically acceptable excipient, wherein the TGF-β antibody comprises the heavy chain and light chain set out in amino acid sequence of SEQ ID NOs: 9 and 10, respectively.

54. The pharmaceutical composition of claim 53, wherein the TGF-β antibody is present at a concentration of 100 mg/mL.

55. The pharmaceutical composition of claim 53 or 54, further comprising (a) a histidine buffer at a concentration of 20 mM with a pH of 5.5; (b) sucrose at a concentration of 220 mM; and (c) a surfactant or polysorbate 20 present at a concentration of 0.04% (w/w).
